(19) United States
(12) Patent Application Publication
(10) Pub. No.: US 2021/0324076 A1
(43) Pub. Date: Oct. 21, 2021

(54) CD33×CD3 BINDING PROTEINS FOR TREATING INFLAMMATORY CONDITIONS AND DISEASES

(71) Applicant: Amphivena Therapeutics, Inc., South San Francisco, CA (US)

(72) Inventors: Jeanmarie Guenot, South San Francisco, CA (US); Eric Feldman, South San Francisco, CA (US); Tae Han, South San Francisco, CA (US)

(21) Appl. No.: 17/284,730

(22) PCT Filed: Oct. 11, 2019
(86) PCT No.: PCT/US2019/055930

§ 371 Date: Apr. 12, 2021

Related U.S. Application Data
(60) Provisional application No. 62/745,247, filed on Oct. 12, 2018.

(51) Int. Cl. C07K 16/28; A61K 39/395

(52) U.S. Cl. CPC C07K 16/2803; C07K 16/2809; A61K 39/3955; A61K 2039/505; C07K 2317/56; C07K 2317/31; C07K 2317/92; C07K 2317/622

(57) ABSTRACT

Described herein are bispecific binding proteins that specifically bind to an antigen expressed on a target cell and an antigen expressed on a T-cell, e.g., human CD33 and human CD3 and therapeutically effective dosing regimens for the treatment and amelioration of an inflammatory disease and condition.

### BACKGROUND

CD33 is a transmembrane cell surface glycoprotein receptor that is specific for myeloid cells. The CD33 antigen is expressed on approximately 90% of AML myeloblasts, including leukemic stem cells and on cells of other myeloproliferative disorders. Myeloid derived suppressor cells (MDSCs), a heterogeneous population of cells involved in immune regulation, also express the CD33 antigen.

### SUMMARY

Provided herein, in one aspect, is a method for the treatment of an inflammatory disease or condition in a subject comprising administering to a subject in need thereof, a protein that binds to human CD33 and human CD3.

In some embodiments, the inflammatory disease or condition is an autoimmune disease. In certain instances, the rheumatoid arthritis, psoriatic arthritis, osteoarthritis, Still's disease, juvenile idiopathic arthritis, lupus, diabetes, myasthenia gravis, Hashimoto's thyroiditis, Ord's thyroiditis, Graves' disease Sjogren's syndrome, multiple sclerosis, Guillain-Barre syndrome, acute disseminated encephalomyelitis, Addison's disease, opsoclonus-myoclonus syndrome, ankylosing spondylitis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia.

In some embodiments, the inflammatory disease or condition is a heteroimmune condition or disease. In certain instances, the heteroimmune condition or disease is graft versus host disease, transplantation rejection, transfusion rejection, anaphylaxis, allergy, type I hypersensitivity, allergic conjunctivitis, allergic rhinitis, and atopic dermatitis.

In some embodiments, the inflammatory disease or condition is inflammatory bowel disease (IBD). In certain instances, the IBD is Crohn's disease or ulcerative colitis.

In some embodiments, the inflammatory disease or condition isasthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gout, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, sepsis, salpingitis, sinusitis, stomatitis, synovitis, tendonitis, tonsillitis, uveitis, vaginitis, vasculitis, and vulvitis.

In some embodiments, the inflammatory disease or condition is caused by a pathogenic infection. In certain instances, the infection is viral, bacterial, or fungal. In some embodiments, the inflammatory disease or condition is caused by an infectious disease. In certain instances, the the infectious disease is hepatitis, HIV, or meningitis.

In some embodiments, the protein is administered at a dose and frequency sufficient to reduce or eliminate myeloid derived suppressor cells (MDSCs).

In some embodiments, the protein is administered as a continuous dose, an intermittent dose, a single dose, multiple doses, or a combination thereof. In other embodiments, the protein is administered as a continuous dose of about 0.5 μg to about 3000 μg per day. In yet other embodiments, the the administration is over a period of time of at least 1 day, at least 1 week, at least 2 weeks, at least 3 weeks, at least 4 weeks, at least 6 weeks, at least 8 weeks, or at least 12 weeks.

In some embodiments, the administration provides a Cₘₐₓ, of about 20 pg/mL to about 10000 pg/mL. In other embodiments, the administration provides a Cₛₛ of about 20 pg/mL to about 10000 pg/mL. In other embodiments, the administration provides an AUC of about 200 day*pg/mL to about 100000 day*pg/mL.

In some embodiments, the administration is intravenous, intramuscular, intralesional, topical or subcutaneous. In some embodiments, the administration is by bolus or continuous infusion.

In some embodiments, the administration provides for gradual T-cell or monocyte activation over 1 to 21 days. In other embodiments, the administration provides for gradual cytokine release over 1 to 21 days. In certain instances, the cytokine is TNFα, IL-2, IL-4, IL-6, IL-8, IL-10, TGF-β, or IFNγ.

In further embodiments, the administration reduces C-reactive protein levels. In certain instances, the administration increases the levels of monocytes, macrophages, neutrophils, basophils, eosinophils, erythrocytes, dendritic cells, megakaryocytes, or platelets. In other instances, the administration increases neutrophil levels. In other instances, the administration increases erythrocyte levels.

In any of the above aspects, the protein is an antibody or antibody derivative. In any of the above aspects, the protein comprises Fab, Fab′, or F(ab′)₂ fragments. In any of the above aspects, the protein comprises a single-chain Fv, tandem single-chain Fv, bi-specific T-cell engager, dual affinity retargeting antibody, diabody, single domain antibody, a bispecific antibody, a bivalent, bispecific (2×2) T-cell engager or a tandem diabody.

In any of the above aspects, the protein is a bivalent, bispecific (2×2) T-cell engager. In some embodiments, the bivalent, bispecific (2×2) T-cell engager comprises a first polypeptide and a second polypeptide, each polypeptide having at least four variable chain domains linked one after another, wherein each polypeptide comprise
(i) a variable heavy chain (VH) domain specific to human CD33;
(ii) a variable light chain (VL) domain specific to human CD33;
(iii) a VH domain specific for human CD3, and
(iv) a VL domain specific for human CD3.

In some instances, in each polypeptide, the four variable chain domains are linked with one after another by peptide linkers L1, L2 and L3 in the order of:
VL(CD3)-L1-VH(CD33)-L2-VL(CD33)-L3-VH(CD3);
VH(CD3)-L1-VL(CD33)-L2-VH(CD33)-L3-VL(CD3);
VL(CD33)-L1-VH(CD3)-L2-VL(CD3)-L3-VH(CD33); or
VH(CD33)-L1-VL(CD3)-L2-VH(CD3)-L3-VL(CD33).

In some instances, the VL domain specific to human CD33 comprises a CDR1 consisting of the sequence selected from the group consisting of SEQ ID NOs:21-27, a CDR2 consisting of the sequence selected from the group consisting of SEQ ID NOs:28-34 and a CDR3 consisting of the sequence of the group consisting of SEQ ID NOs:35-41.

In some instances, the VH domain specific to human CD33 comprises a CDR1 consisting of the sequence selected from the group consisting of SEQ ID NOs:42-48, a CDR2 consisting of the sequence selected from the group consisting of SEQ ID NOs:49-55 and a CDR3 consisting of a sequences selected from the group consisting of SEQ ID NOs:56-63.

In some instances, the CDR1, CDR2 and CDR3 of the VL domain specific to human CD33 are sequences selected from the group consisting of:
(i) SEQ ID NOs:21, 28 and 35;
(ii) SEQ ID NOs:22, 29 and 36;
(iii) SEQ ID NOs:23, 30 and 37;
(iv) SEQ ID NOs:24, 31 and 38;
(v) SEQ ID NOs:25, 32 and 39;
(vi) SEQ ID NOs:26, 33 and 40; and
(vii) SEQ ID NOs:27, 34 and 41.

In some instances, the CDR1, CDR2 and CDR3 of the VH domain specific to CD33 are sequences selected from the group consisting of:
(i) SEQ ID NOs:42, 49 and 56;
(ii) SEQ ID NOs:43, 50 and 57;
(iii) SEQ ID NOs:43, 50 and 58;
(iv) SEQ ID NOs:43, 50 and 59;
(v) SEQ ID NOs:43, 50 and 60;
(vi) SEQ ID NOs:44, 51 and 61;
(vii) SEQ ID NOs:45, 52 and 62;
(viii) SEQ ID NOs:46, 53 and 63;
(ix) SEQ ID NOs:47, 54 and 63; and
(x) SEQ ID NOs:48, 55 and 63.

In some instances, the VL and VH domains specific to CD33 are sequences selected from the group consisting of:
(i) SEQ ID NO:1 and SEQ ID NO:11;
(ii) SEQ ID NO:2 and SEQ ID NO: 12,
(iii) SEQ ID NO:3 and SEQ ID NO: 13;
(iv) SEQ ID NO:4 and SEQ ID NO: 14,
(v) SEQ ID NO:5 and SEQ ID NO:15;
(vi) SEQ ID NO:6 and SEQ ID NO: 16,
(vii) SEQ ID NO:7 and SEQ ID NO: 17;
(viii) SEQ ID NO:8 and SEQ ID NO: 18.
(ix) SEQ ID NO:9 and SEQ ID NO: 19; and
(x) SEQ ID NO:10 and SEQ ID NO:20.

In some instances, the VH domain specific for human CD3 comprises a CDR1 sequence of STYAMN (SEQ ID NO:72), a CDR2 sequence of RIRSKYNNYATYYADSVKD (SEQ ID NO:73) and a CDR3 sequence of HGNFGNSYVSWFAY (SEQ ID NO:74) or HGNFGNSYVSYFAY (SEQ ID NO:75).

In some instances, the VL domain specific for human CD3 comprises a CDR1 sequence of RSSTGAVTTSNYAN (SEQ ID NO:90), a CDR2 sequence of GTNKRAP (SEQ ID NO:91), and a CDR3 sequence of ALWYSNL (SEQ ID NO:92).

In some instances, the VL and VH domains specific to CD3 are sequences selected from the group consisting of:
(i) SEQ ID NO:64 and SEQ ID NO:68;
(ii) SEQ ID NO:65 and SEQ ID NO:69;
(iii) SEQ ID NO:66 and SEQ ID NO:70; and
(iv) SEQ ID NO:67 and SEQ ID NO:71.

In some instances, each polypeptide comprises four variable chain domains selected from the group consisting of:
(i) SEQ ID NOs:2, 12, 65 and 69;
(ii) SEQ ID NOs:3, 13, 65 and 69;
(iii) SEQ ID NOs:4, 14, 65 and 69;
(iv) SEQ ID NOs:5, 15, 65 and 69;
(v) SEQ ID NOs:1, 11, 64 and 68;
(vi) SEQ ID NOs:2, 12, 64 and 68;
(vii) SEQ ID NOs:2, 12, 66 and 70;
(viii) SEQ ID NOs:4, 14, 66 and 70;
(ix) SEQ ID NOs:5, 15, 66 and 70;
(x) SEQ ID NOs:3, 13, 64 and 68;
(xi) SEQ ID NOs:3, 13, 67 and 71;
(xii) SEQ ID NOs:4, 14, 64 and 68;
(xiii) SEQ ID NOs:5, 15, 64 and 68;
(xiv) SEQ ID NOs:7, 17, 64 and 68;
(xv) SEQ ID NOs:6, 16, 64 and 68;
(xvi) SEQ ID NOs:6, 16, 67 and 71;
(xvii) SEQ ID NOs:8, 18, 64 and 68;
(xviii) SEQ ID NOs:9, 19, 64 and 68;
(xix) SEQ ID NOs:9, 19, 67 and 71; and
(xx) SEQ ID NOs:10, 20, 64 and 68.

In some instances, the bivalent, bispecific (2-2) T-cell engager comprises a sequence selected from the group consisting of SEQ ID NOs:98-121. In some instances, the bivalent, bispecific (2×2) T-cell engager comprises a sequence selected from the group consisting of SEQ ID Nos 123-146.

### BRIEF DESCRIPTION OF THE DRAWINGS

FIG. 1 Schematic representation of the gene organization and a domain order of a CD3/CD33 bivalent, bispecific (2×2) T-cell engager. Exemplary 2×2 T-cell engagers are expressed as a single polypeptide comprised of four variable domains connected via short peptide linkers L1, L2 and L3. Following expression, two monomeric polypeptides associate non-covalently head-to-tail to form the functional homodimeric molecule. L1, L2, L3: Linker; V_{H}: Heavy chain variable domain; V_{L}: Light chain variable domain.

FIG. 2 CD3 engaging 2×2 T-cell engager and its mode of action. 2×2 T-cell engagers are bispecific proteins with two binding sites for each antigen that leads to activation and proliferation of T-cells. A CD33/CD3 2.2 T-cell engager binds to a CD33 tumor cell with two of four binding domains and to CD3 with the other two binding domains. This T-cell/target cell binding forms an immunological synapse that promotes activation of the T-cell and promotes the subsequent destruction of the tumor cell via apoptosis.

FIG. 3 Domain order variants of CD33/CD3 2×2 T-cell engagers. Variations of domain order of variable heavy (VH) and variable light (VL) chains within gene sequences encoding 2×2 T-cell engagers allows production of antibodies with CD33 and CD3 specificities located on the inside or outside of the molecule. Domain specificities, location of signal sequences (ss) and linkers (L1, L2, L3) and affinity tags (His) as well as 5′- and 3′-ends are indicated.

FIG. 4 Comparison of positively enriched vs. negatively selected healthy donor T-cells. KG-1a cells were incubated with 10 pM (approx. 1 ng/mL) and 25 pM (approx. 2.5 ng/mL) of one of 10 selected 2×2 T-cell engagers and either negatively selected healthy donor T-cells or positively selected healthy donor T-cells at an E:T cell ratio of 1:1 or 3:1, as indicated. After 48 hours, cell counts were determined and cytotoxicity was assessed with DAPI staining. Results are shown as mean±SEM for the percentage of dead cells (upper panels) and the percentage of specific cytotoxicity (lower panels) from 3 independent experiments performed in duplicate wells.

FIG. 5 Analysis strategy. Scatter and histogram plots from one healthy donor T-cell aliquot and 1 representative AML cell line (HL-60) and primary AML specimen (AMP002) each illustrating the strategy pursued to determine 2×2 T-cell engager-induced cytotoxicity. FSC, forward scatter; SSC, side scatter.

FIGS. 6A-D Screening cytotoxicity assays in CD33+ AML cell lines. Parental HL-60 (A,B) and KG-1a (C,D) cells were incubated with 10 pM (approx. 1 ng/mL) and 25 pM (approx. 2.5 ng/mL) of one of 22 CD33/CD3 2-2 T-cell engager molecules or a non-binding control 2×2 T-cell engager (00) and healthy donor T-cells at an E:T cell ratio of either 1:1 (A,C) or 5:1 (B,D) as indicated. After 48 hours, cell counts were determined and cytotoxicity was assessed with DAPI staining to quantify drug-specific cytotoxicity. Results are shown as mean±SEM for the percentage of DAPI⁺ cells from 3 independent experiments performed in duplicate wells. Qualitatively similar results were obtained when cytotoxicity was expressed as the percentage of specific cytotoxicity.

FIG. 7 Selection of primary AML specimens for study. Frozen aliquots from a total of primary human AML specimens were obtained for analysis. The percentage of AML blasts upon thaw was determined by flow cytometry based on CD45/side-scatter properties. Viability of the specimens was determined upon thaw as well after 48 hours in cytokine-containing liquid culture (without addition of 2×2 T-cell engager molecules or healthy donor T-cells) via flow cytometry using DAPI as live/dead cell marker. Results for viability after thawing as well as after 48 hours are depicted for all specimens, which had >58% AML blasts. Square: Primary AML specimens that showed a viability of >50% at thaw as well as >50% after 48 hours in cytokine-containing liquid culture which were included in the final analyses.

FIGS. 8A-C 2×2 T-cell engager-induced cytotoxicity in primary AML specimens. Primary AML specimens were incubated with 2.5 pM (approx. 250 pg/mL), 10 pM (approx. 1 ng/mL), and 25 pM (approx. 2.5 ng/mL) of one of 9 2×2 T-cell engager molecules without healthy donor T-cells added (A) or with healthy donor T-cells at an E:T cell ratio of either 1:3 (B) or 1:1 (C) as indicated. After 48 hours, cell counts were determined and cytotoxicity was assessed with DAPI staining to quantify drug-specific cytotoxicity. Results are shown as mean±SEM for the percentage of specific cytotoxicity from experiments performed in duplicate wells.

FIG. 9 Amino acid sequence of extracellular domain of human CD33 (aa 18-259) (SEQ ID NO: 93);

FIGS. 10A-X Amino acid sequences
(A) complete sequence of 2×2 T-cell engager 1 with C-terminal hexa-histidine (6×His)-tag (SEQ ID NO:98);
(B) complete sequence of 2×2 T-cell engager 2 with C-terminal hexa-histidine (6×His)-tag (SEQ ID NO:99);
(C) complete sequence of 2×2 T-cell engager 3 with C-terminal hexa-histidine (6×His)-tag (SEQ ID NO:100);
(D) complete sequence of 2×2 T-cell engager 4 with C-terminal hexa-histidine (6×His)-tag (SEQ ID NO:101);
(E) complete sequence of 2×2 T-cell engager 5 with C-terminal hexa-histidine (6×His)-tag (SEQ ID NO:102);
(F) complete sequence of 2×2 T-cell engager 6 with C-terminal hexa-histidine (6×His)-tag (SEQ ID NO:103);
(G) complete sequence of 2×2 T-cell engager 7 with C-terminal hexa-histidine (6×His)-tag (SEQ ID NO:104);
(H) complete sequence of 2×2 T-cell engager 8 with C-terminal hexa-histidine (6×His)-tag (SEQ ID NO:105);
(I) complete sequence of 2×2 T-cell engager 9 with C-terminal hexa-histidine (6×His)-tag (SEQ ID NO:106);
(J) complete sequence of 2×2 T-cell engager 10 with C-terminal hexa-histidine (6×His)-tag (SEQ ID NO:107);
(K) complete sequence of 2×2 T-cell engager 11 with C-terminal hexa-histidine (6×His)-tag (SEQ ID NO: 108);
(L) complete sequence of 2×2 T-cell engager 12 with C-terminal hexa-histidine (6×His)-tag (SEQ ID NO:109);
(M) complete sequence of 2×2 T-cell engager 13 with C-terminal hexa-histidine (6×His)-tag (SEQ ID NO:110);
(N) complete sequence of 2×2 T-cell engager 14 with C-terminal hexa-histidine (6×His)-tag (SEQ ID NO:111);
(O) complete sequence of 2×2 T-cell engager 15 with C-terminal hexa-histidine (6×His)-tag (SEQ ID NO:112);
(P) complete sequence of 2×2 T-cell engager 16 with C-terminal hexa-histidine (6×His)-tag (SEQ ID NO:113);
(Q) complete sequence of 2×2 T-cell engager 17 with C-terminal hexa-histidine (6×His)-tag (SEQ ID NO:114);
(R) complete sequence of 2×2 T-cell engager 18 with C-terminal hexa-histidine (6×His)-tag (SEQ ID NO:115);
(S) complete sequence of 2×2 T-cell engager 19 with C-terminal hexa-histidine (6×His)-tag (SEQ ID NO: 116);
(T) complete sequence of 2×2 T-cell engager 20 with C-terminal hexa-histidine (6×His)-tag (SEQ ID NO: 117);
(U) complete sequence of 2-2 T-cell engager 21 with C-terminal hexa-histidine (6×His)-tag (SEQ ID NO:118);
(V) complete sequence of 2×2 T-cell engager 22 with C-terminal hexa-histidine (6×His)-tag (SEQ ID NO:119);
(W) complete sequence of 2×2 T-cell engager 23 with C-terminal hexa-histidine (6×His)-tag (SEQ ID NO:120); and
(X) complete sequence of 2×2 T-cell engager 24 with C-terminal hexa-histidine (6×His)-tag (SEQ ID NO:121). Underlined sequences represent linkers L1, L2 and L3.

FIGS. 11A-X Amino acid sequences
(A) complete sequence of 2×2 T-cell engager 1 (SEQ ID NO: 123),
(B) complete sequence of 2×2 T-cell engager 2 (SEQ ID NO: 124);
(C) complete sequence of 2×2 T-cell engager 3 (SEQ ID NO: 125);
(D) complete sequence of 2×2 T-cell engager 4 (SEQ ID NO:126);
(E) complete sequence of 2×2 T-cell engager 5 (SEQ ID NO: 127);
(F) complete sequence of 2×2 T-cell engager 6 (SEQ ID NO: 128);
(G) complete sequence of 2×2 T-cell engager 7 (SEQ ID NO: 129);
(H) complete sequence of 2×2 T-cell engager 8 (SEQ ID NO: 130);
(I) complete sequence of 2×2 T-cell engager 9 (SEQ ID NO:131);
(J) complete sequence of 2×2 T-cell engager 10 (SEQ ID NO:132);
(K) complete sequence of 2×2 T-cell engager 11 (SEQ ID NO:133);
(L) complete sequence of 2×2 T-cell engager 12 (SEQ ID NO:134);
(M) complete sequence of 2×2 T-cell engager 13 (SEQ ID NO:135);
(N) complete sequence of 2×2 T-cell engager 14 (SEQ ID NO:136);
(O) complete sequence of 2×2 T-cell engager 15 (SEQ ID NO:137);
(P) complete sequence of 2×2 T-cell engager 16 (SEQ ID NO:138);
(Q) complete sequence of 2×2 T-cell engager 17 (SEQ ID NO:139);
(R) complete sequence of 2×2 T-cell engager 18 (SEQ ID NO:140);
(S) complete sequence of 2×2 T-cell engager 19 (SEQ ID NO:141);
(T) complete sequence of 2-2 T-cell engager 20 (SEQ ID NO: 142);
(U) complete sequence of 2×2 T-cell engager 21 (SEQ ID NO:143);
(V) complete sequence of 2-2 T-cell engager 22 (SEQ ID NO: 144);
(W) complete sequence of 2×2 T-cell engager 23 (SEQ ID NO: 145); and
(X) complete sequence of 2×2 T-cell engager 24 (SEQ ID NO:146). Underlined sequences represent linkers L1, L2 and L3.

FIG. 12 Effect of 2×2 T-cell engagers 16 and 12 on the growth of HL-60 cells in NOD/scid mice. Eight experimental groups of immunodeficient NOD/scid mice were xenotransplanted by subcutaneous injection with a suspension of 4×10⁶ HL-60 cells on day 0. Prior to injection HL-60 cells were mixed with 3×10⁶ purified T-cells from healthy donors. All animals of the experimental groups transplanted with tumor cells and T-cells received an intravenous bolus on days 0, 1, 2, 3 and 4 of either vehicle (control) or 2×2 T-cell engagers 16 or 12 at three different dose levels as indicated (0.1 μg, 1 μg, and 10 μg). One group without effector cells and vehicle treatment served as an additional negative control.

FIG. 13 Anti-tumor activity of 2×2 T-cell engager 16 in an AML Xenograft Model. NOD/scid mice were sublethally irradiated (2 Gy) and subcutaneously inoculated with 4×10⁶ HL-60 cells. On day 9 the animals received a single bolus injection of anti-asialo GM1 rabbit Ab. When tumors reached a volume between 50-150 mm³ (mean 73 f 11 mm³) on day 10 animals were allocated to 3 treatment groups. Groups 2 and 3 (n=8) were intraperitoneally injected with 1.5×10⁷ expanded and activated human T-cells. From day 13 to day 21 (qdxd9) animals received either 2×2 T-cell engager 16 (Group 3) or vehicle into the lateral tail vein (Group 1 and Group 2).

FIGS. 14A-B Relative amount (A) and absolute counts (B) of human AML blasts in the bone marrow (BM) and spleen of NSG mice at day 38 after treatment with 5 μg (0.25 mg/kg) or 50 μg (2.5 mg/kg) CD33/CD3 2×2 T-cell engager 12 and 16.

FIG. 15 Kinetics of CD33/CD3 2×2 T-cell engager 16-mediated target cell lysis. 1×10⁴ calcein-labeled HL-60 target cells were incubated with primary human T-cells as effector cells at an E:T ratio of 25:1 in the presence of serial dilutions of 2×2 T-cell engager 16 or without antibody (w/o) for 30 min, 1 h, 2 h, 3 h, 4 h, or 5 h. At each time point, the fluorescent calcein released from lysed target cells was used to calculated specific lysis. Mean and SD of three replicates are plotted.

FIG. 16 Kinetics of EC₅₀ and specific lysis values for CD33/CD3 2×2 T-cell engager 16. EC₅₀ values (black solid circles) and 2×2 T-cell engager 16-mediated target cell lysis (open squares) were determined in calcein-release cytotoxicity assays at the indicated incubation times by non-linear regression/sigmoidal dose-response and plotted.

FIG. 17 Cytotoxic activity in newly diagnosed, relapsed and refractory AML patient samples.

FIG. 18 Serum concentration of CD33/CD3 2×2 T-cell engager 16 in subjects 02-001, -002, and -003 at a dose of 0.5 μg/day.

FIG. 19 Serum concentration of CD33/CD3 2×2 T-cell engager 16 for 14 days at denoted dose levels to patients with to patients with relapsed/refractory acute myeloid leukemia.

FIG. 20 Exemplary levels of myeloblasts (upper left panel), absolute neutrophil counts (upper right panel), hemoglobin (lower left panel) and C-reactive protein (CRP) (lower right panel) in a subject dosed with CD33/CD3 2×2 T-cell engager 16 a dose of 0.5 μg/day for 14 days according to Example 14.

FIG. 21 Exemplary levels of blood counts, i.e., red blood cells (upper left panel) and white blood cells (upper right panel), interleukin-6 (lower left panel) and CRP (lower right panel) in a subject dosed with CD33/CD3 2×2 T-cell engager 16 a dose of 1.5 μg/day for 14 days according to Example 14.

FIG. 22 Improved hemoglobin, neutrophils, platelet, and monocyte counts improvement following administration of AMV564 at a 1.5 mcg dose level for 14 days in a subject (upper panels). Improved hemoglobin, neutrophils, and platelet counts as well as decreased CRP levels following administration of AMV564 at a 1.5 mcg dose level for 14 days in a subject (lower panels).

FIG. 23 Best relative change in percent bone marrow leukemic blasts from baseline following administration of AMV564 for 14 days to patients with relapsed/refractory acute myeloid leukemia.

FIG. 24 Exemplary dosing regimens for AMV564. Intermittent dosing every other day with titration from 5 μg→15 μg→100 μg (upper panel). Intermittent dosing with 15 μg continuous infusion×3 days followed by every other day with titration from 100 μg→200 μg.

### DETAILED DESCRIPTION

Described herein are pharmaceutical means and methods for immunological, medical interventions based on administering therapeutic proteins, in particular bispecific antibodies to an antigen expressed on a target cell and an antigen expressed on a T-cell, e.g., CD33 and CD3.

The bispecific antibodies described herein are useful for the prevention, treatment or amelioration of an inflammatory disease or condition by removal or elimination of myeloid derived suppressor cells (MDSCs).

### Myeloid Derived Suppressor Cells

Myeloid derived suppressor cells (MDSCs) are a heterogeneous population of cells involved in immune regulation. MDSCs have been observed in cancers, largely for their role in engineering an immunosuppressive tumor microenvironment, thereby allowing the cancer to evade anti-tumor immune responses. As such, the presence of MDSCs in the tumor microenvironment correlates will with decreased efficacies of certain immunotherapies, including adoptive cell therapies, dendritic cell vaccination, and the like.

MDSCs are a heterogeneous population of immature myeloid cells in the blood that have the morphology of granulocytes or monocytes. In mice, MDCSs are broadly classified into two subgroups: granulocytic MDSCs (Gr-MDSCs) and monocytic MDSCs (mo-MDSCs) which are distinguished by appearance, surface marker expression and mechanism of immunosuppression. In contrast, human MDSCs lack adequate characterization due to a lack of uniform or specific markers. Generally, human MDSCs are defined as having CD33 and CD11b markers, lacking HLA-DR and having either CD14 or CD15.

MDSCs create immunosuppressive effects through a variety of mechanisms, such as the production of reactive oxygen species, nitric oxide, arginase-1, interleukin-10 and transforming growth factor-β. Through these processes, CD4⁺ T-cell response and proliferation is inhibited along with anti-proliferative molecules like interferon-γ. MDSCs have also been observed to down-regulate the function of NK and dendritic cells, immune molecules that have effects on cancer progression. Finally, MDSCs can induce and mediate the expansion of regulatory T (T_{reg}) cells, which enhances immune suppression. In chronic inflammation, MDSCs are expanded and found at inflammation sites to suppress T cell immune function.

Given the importance and role MDSCs have on immune regulation and function, it is therefore an aim of the present embodiments described herein to provide therapeutic agents that target MDSCs, thereby removing immunosuppressive effects in inflammatory conditions and diseases. Specifically, such therapeutic agents comprise proteins and antibodies that bind to CD33 and CD3.

### Bispecific Antibodies to CD33 and CD3

According to a first aspect, described herein are bispecific antibodies having specificity to an antigen expressed on a target cell and an antigen expressed on a T-cell. In some embodiments, the bispecific antibodies have specificity for at least CD33, preferably human CD33. In some embodiments, the CD33 binding domains of the bispecific antibodies described herein have specificity for human and cynomolgus CD33, i.e. are cross-reactive. In some embodiments, these cross-reactive binding domains bind to human and cynomolgus CD33 with similar affinity.

CD33 is a transmembrane cell surface glycoprotein receptor that is specific for myeloid cells. The CD33 antigen is expressed on approximately 90% of acute myeloid leukemia (AML) myeloblasts and cells of other myeloproliferative disorders, including leukemic stem cells and myeloid derived suppressor cells (MDSCs). CD33 is expressed on monocytes, dendritic cells, neutrophils, resident macrophages, basophils and eosinophils. Two alternatively spliced isoforms have been identified that may have implications for downstream signaling.

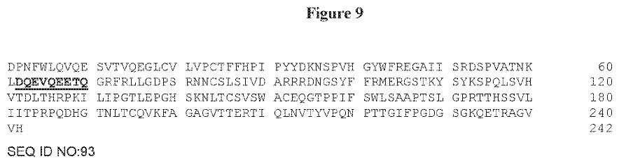

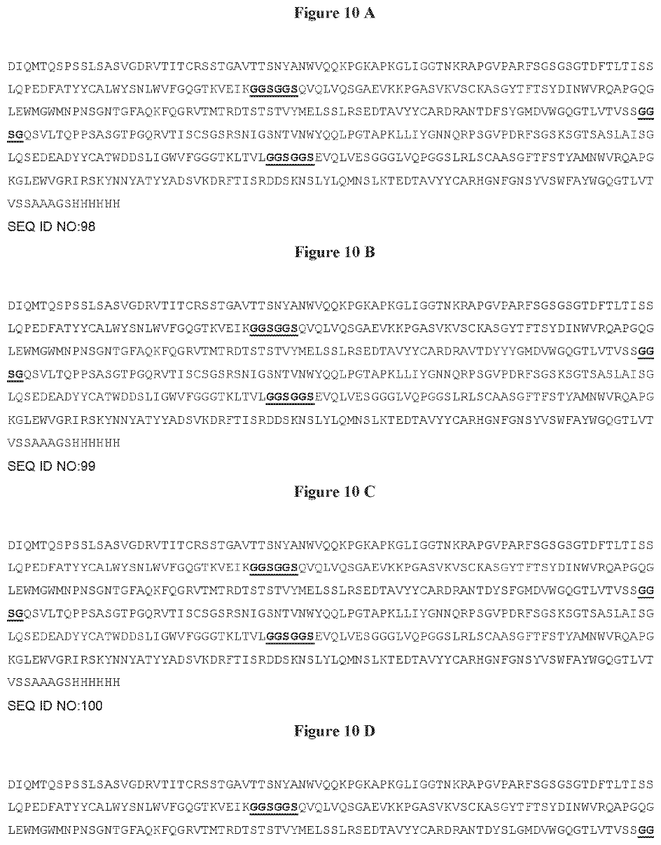

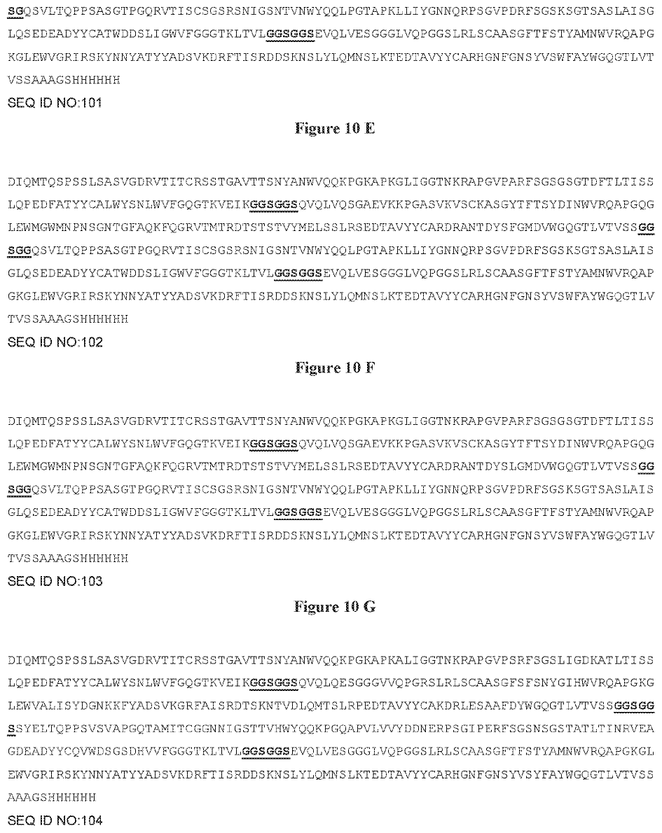

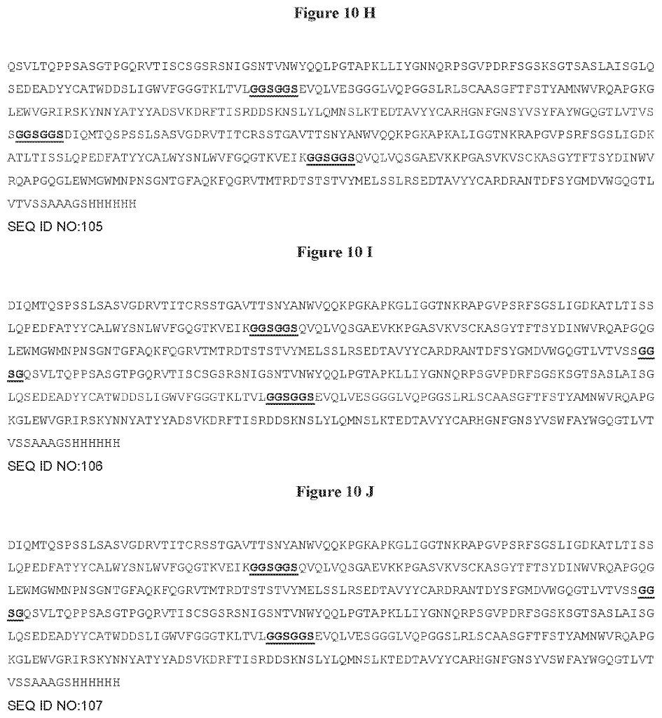

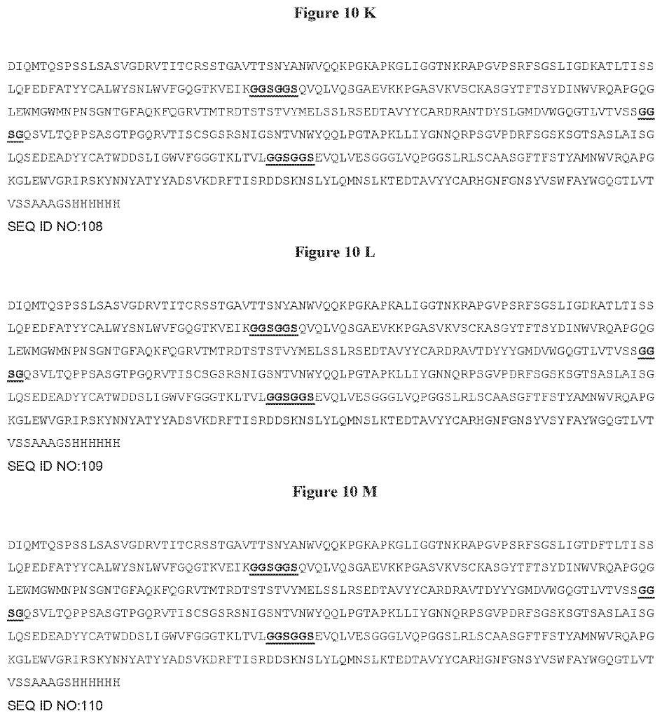

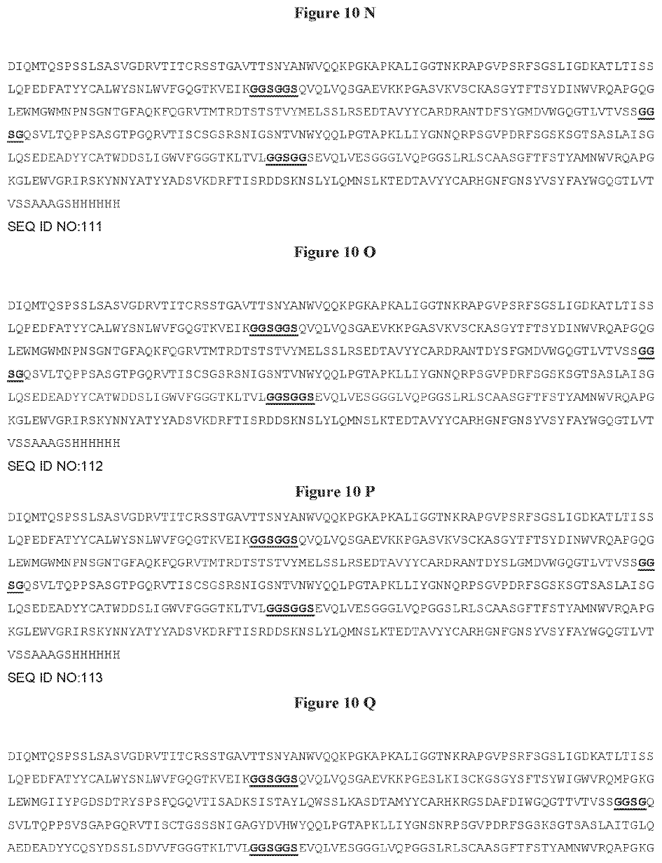

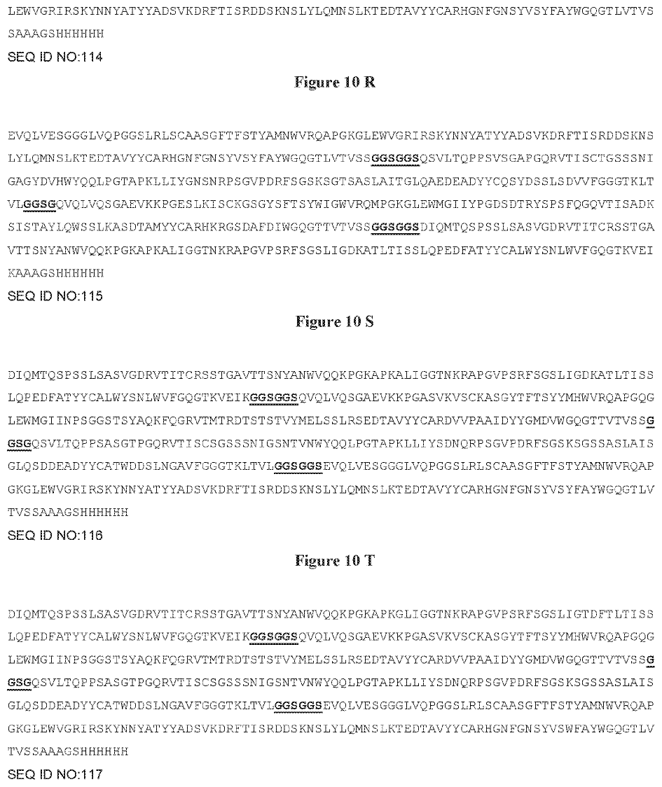

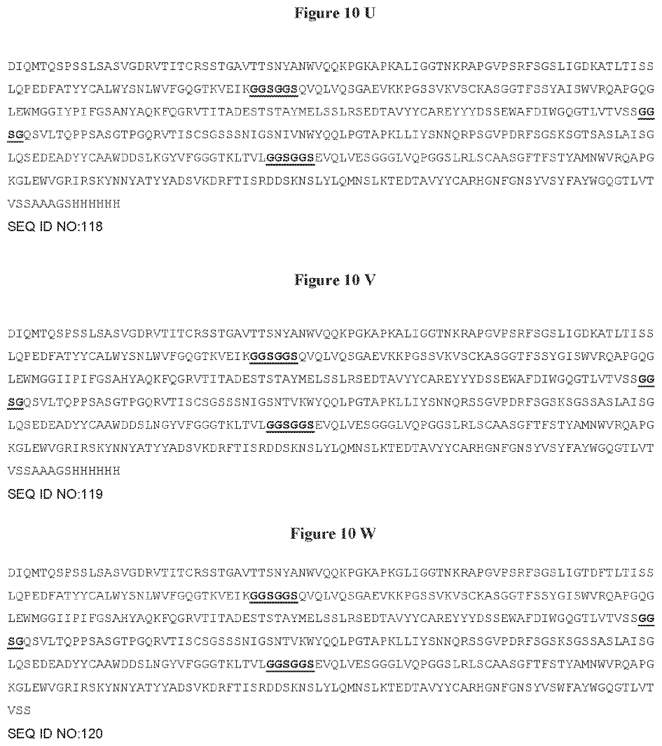

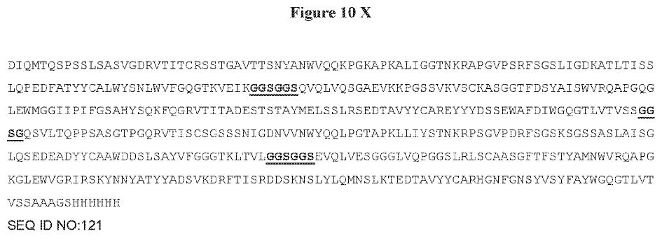

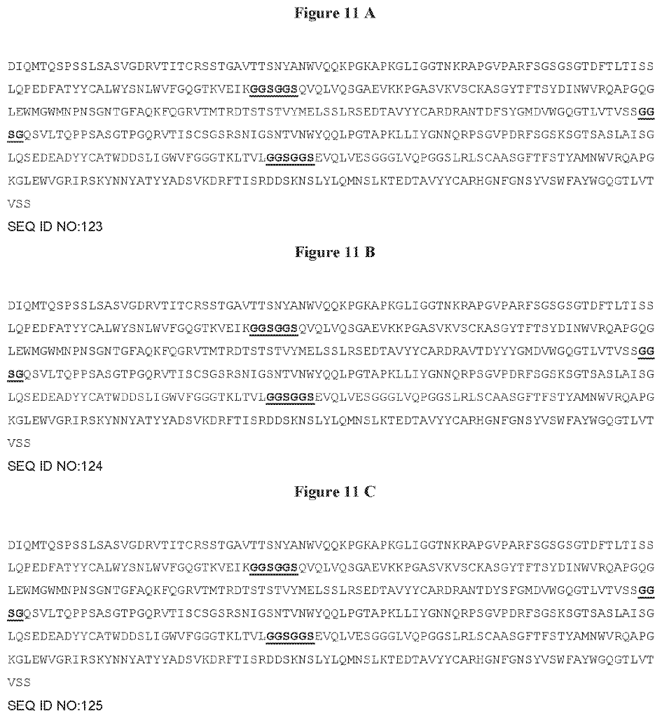

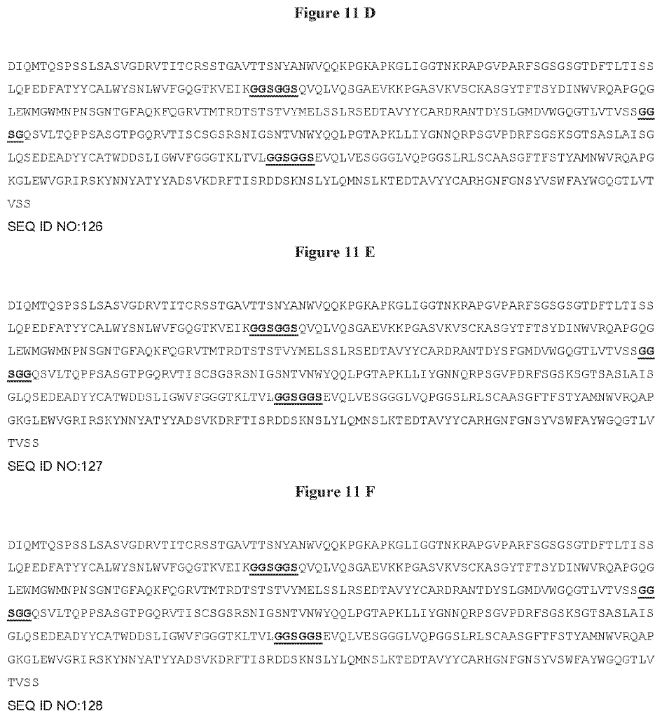

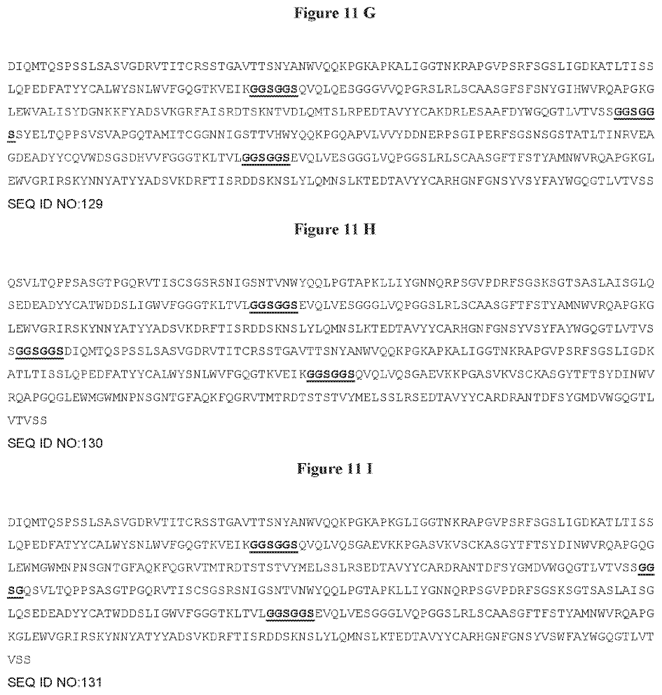

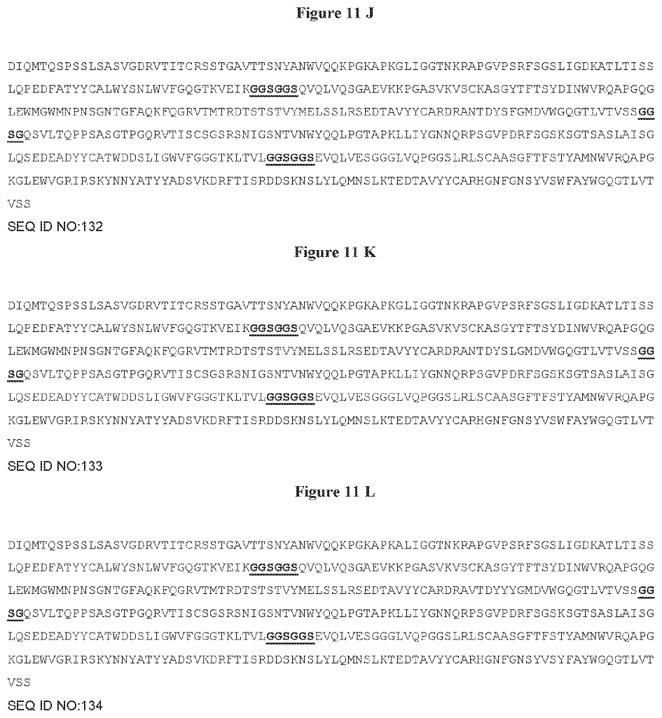

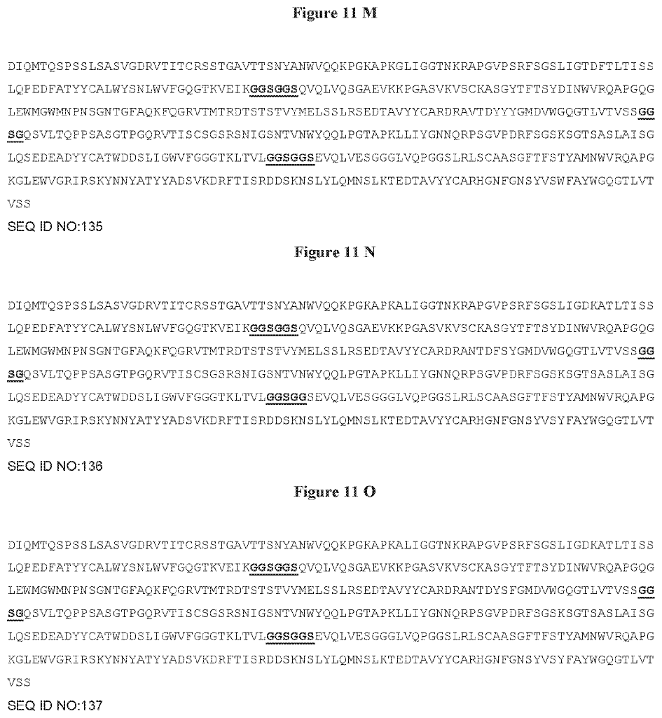

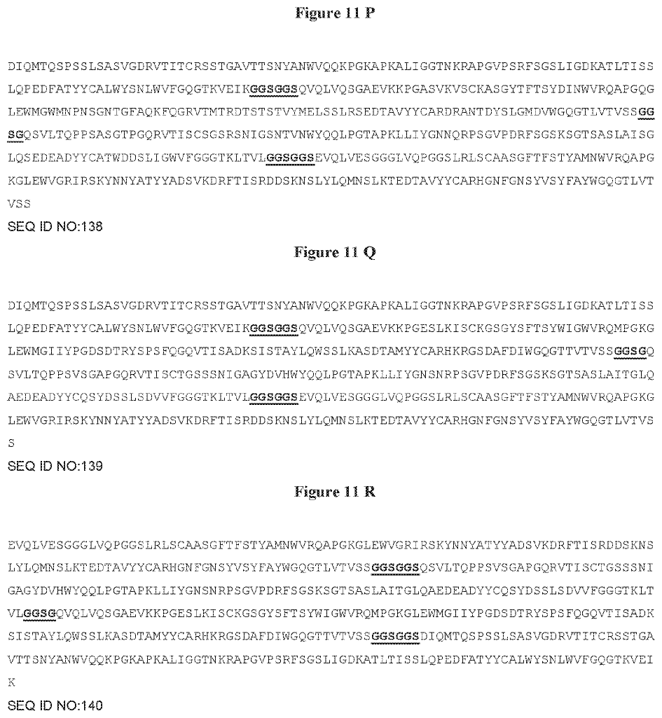

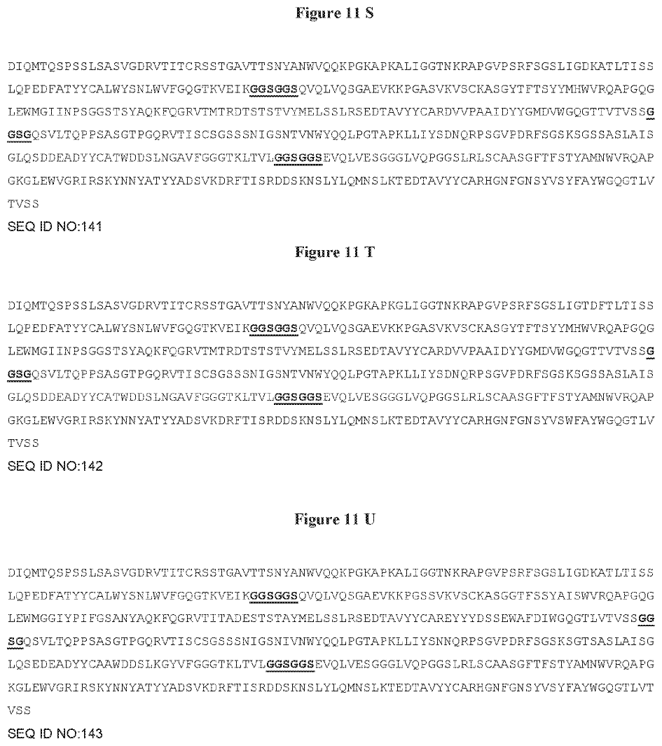

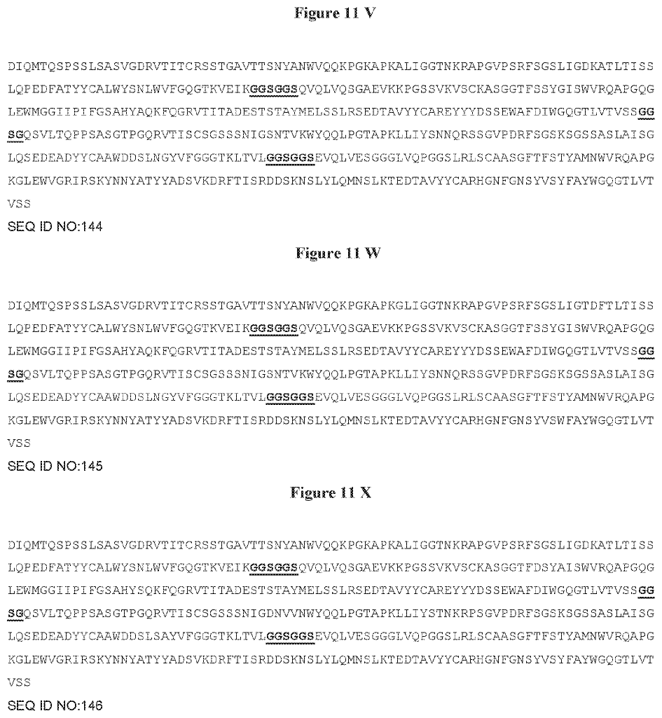

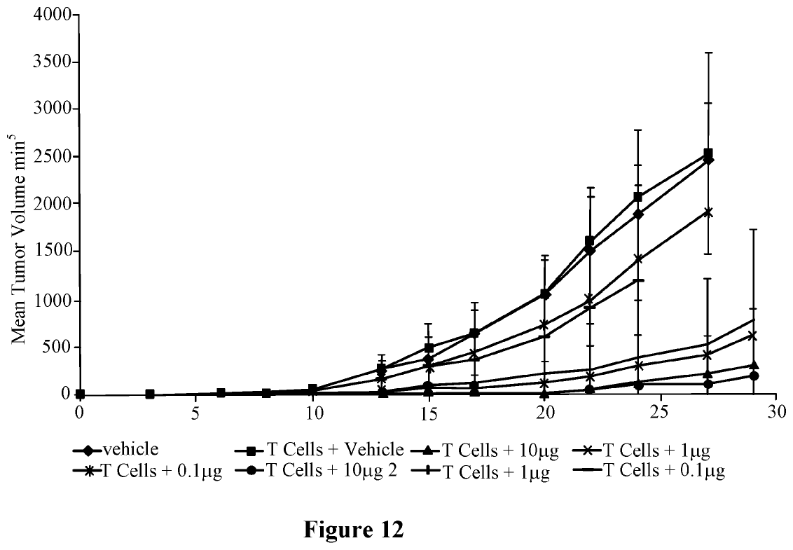

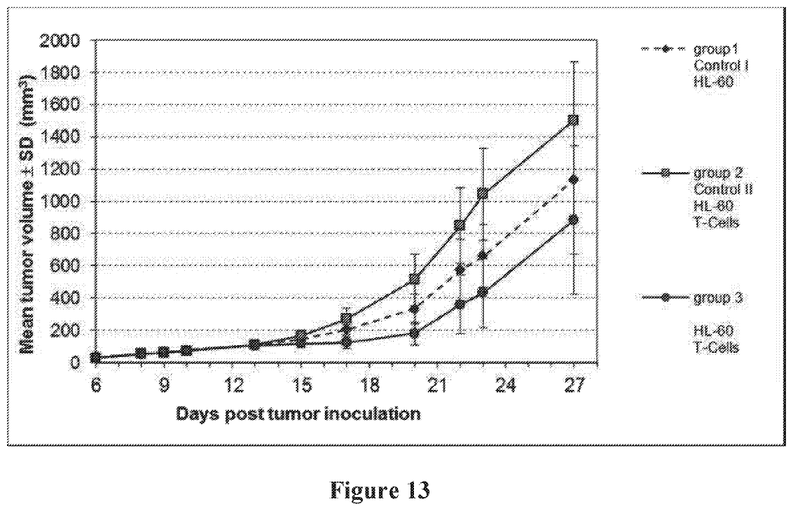

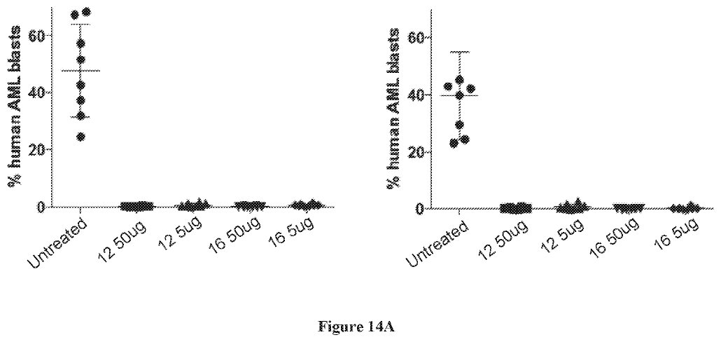

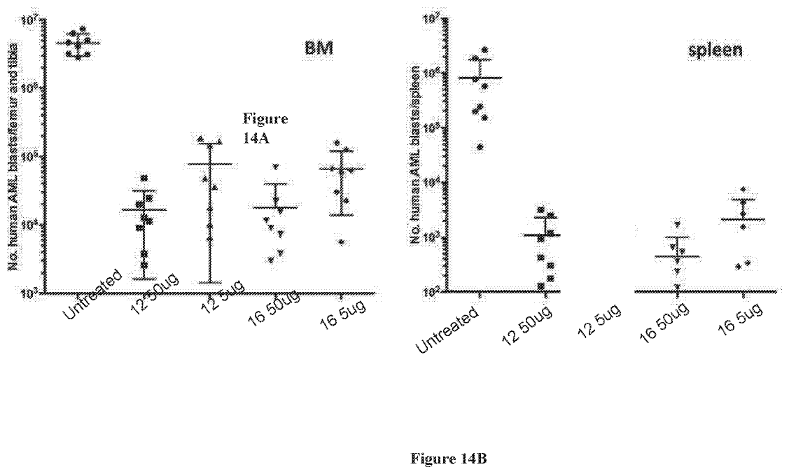

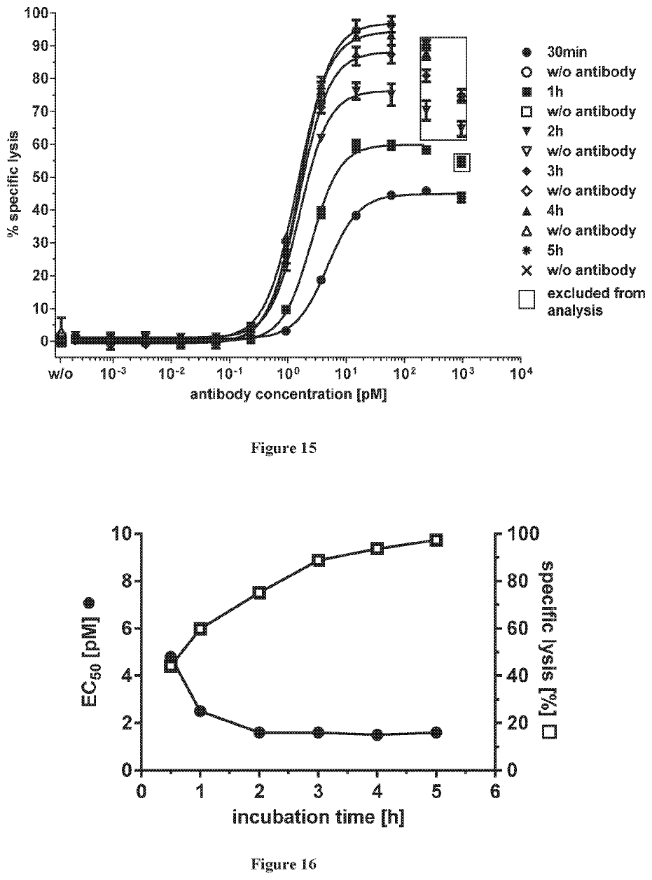

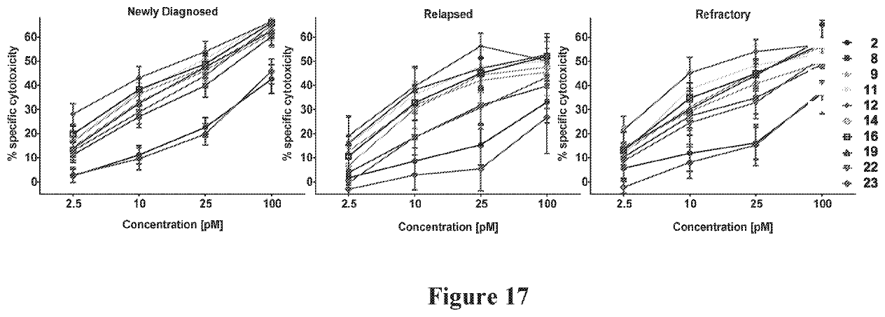

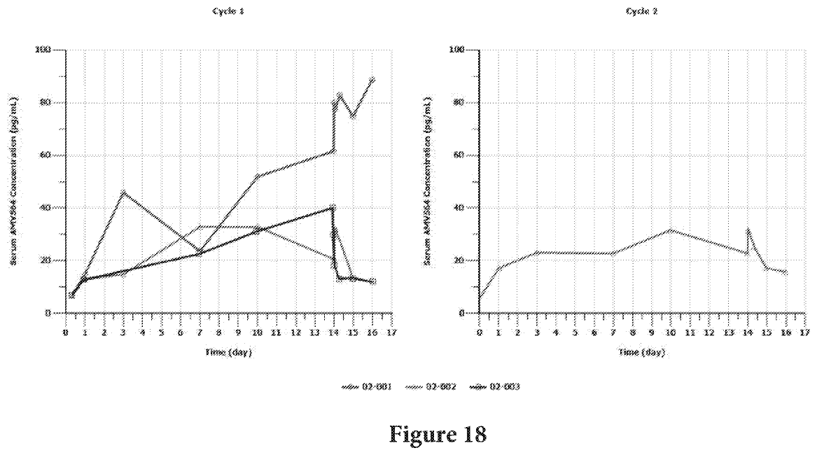

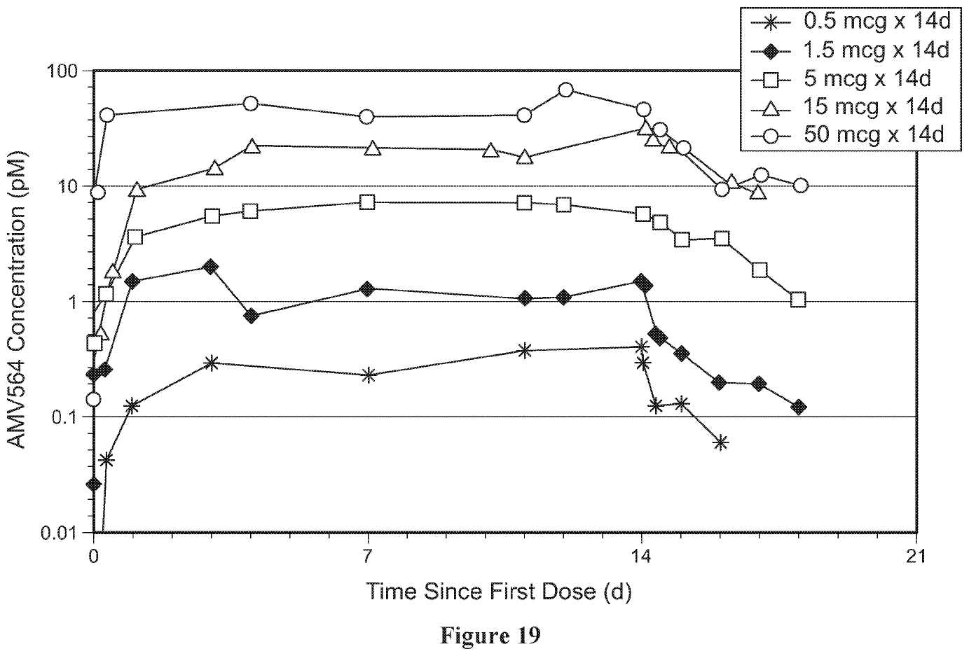

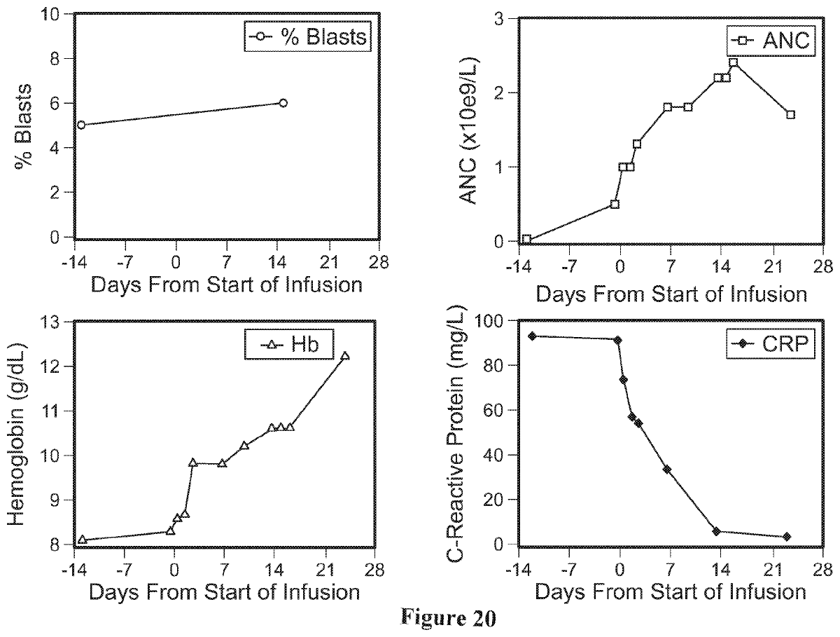

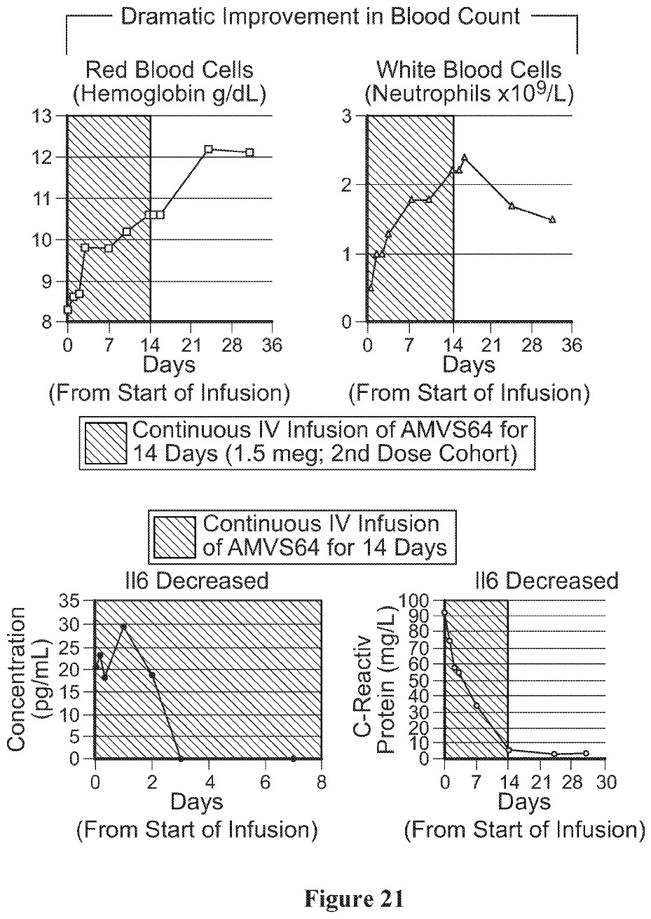

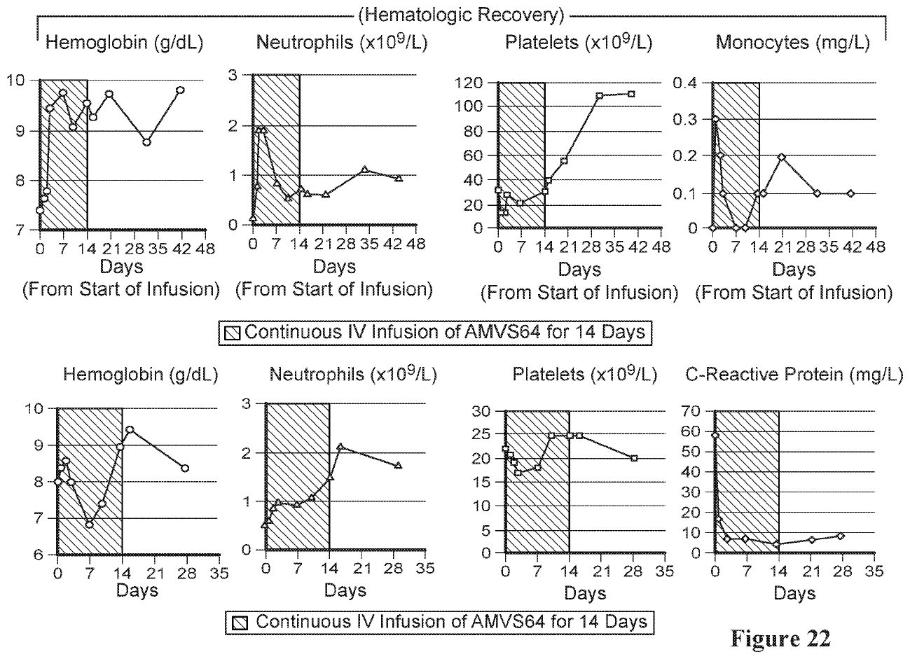

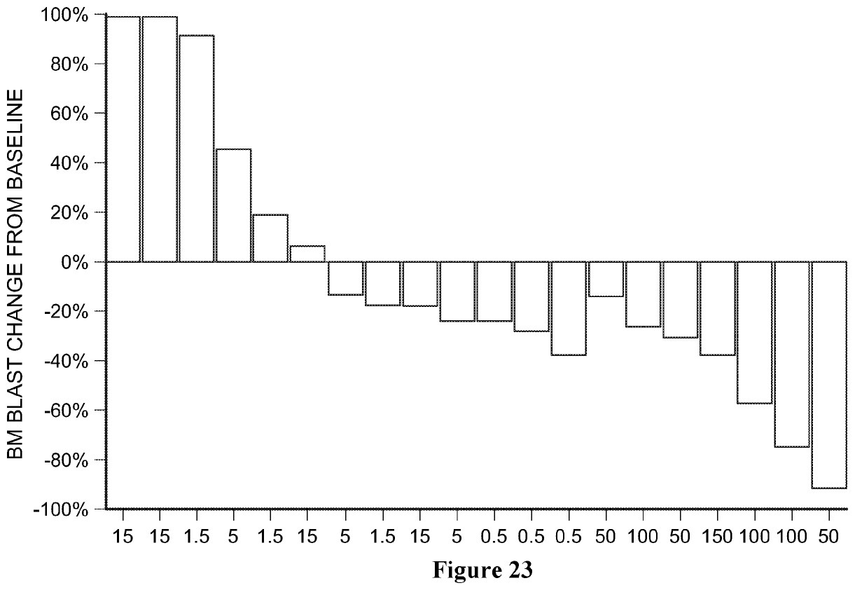

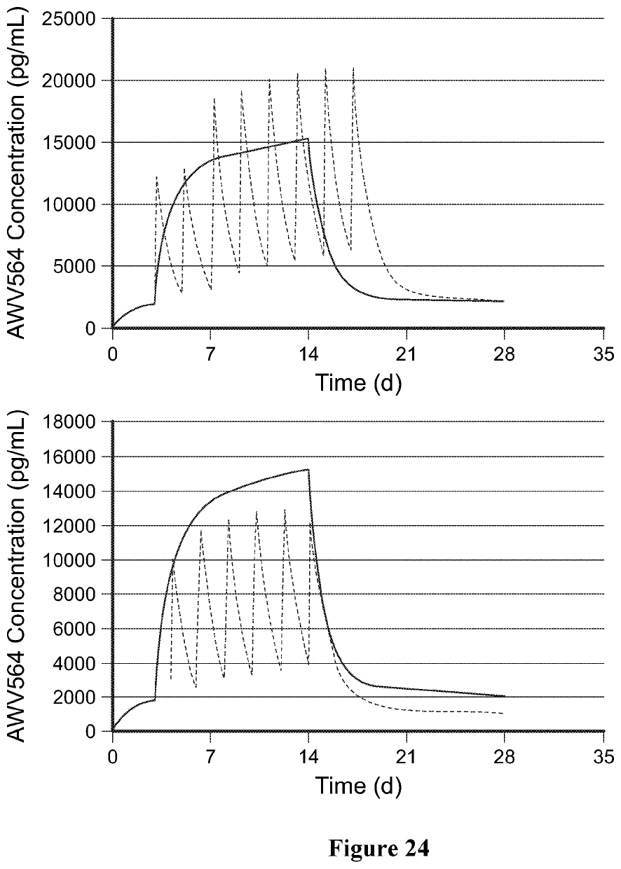

For the isolation of antibody domains specific for CD33, such as human CD33, antibody libraries may be screened. For example IgM phage display libraries can be screened by employing, for example, a recombinant CD33-Fc fusion protein containing amino acids 1-243 of the extracellular domain of human CD33 (FIG. 9, SEQ ID NO:93).

In some embodiments the CD33 binding domain has at least one CD33 binding site comprising a light chain variable domain and a heavy chain variable domain. The light chain variable domain comprises the light chain CDR1, CDR2 and CDR3 and the heavy chain variable domain comprises the heavy chain CDR1, CDR2 and CDR3. In some embodiments these light chain CDRs (CDR1, CDR2 and CDR3) are selected from the human CDR sequences shown in Table 1 (SEQ ID NOs:21-41). In certain instances, the light chain CDR1 is selected from SEQ ID NOs:21-27. In certain instances, the light chain CDR2 is selected from SEQ ID NOs:28-34. In certain instances, the light chain CDR3 is selected from SEQ ID NOs:35-41.

In some embodiments these heavy chain CDRs (heavy chain CDR1, CDR2 and CDR3) are selected from the human CDR sequences shown in Table 2 (SEQ ID NOs:42-63). In certain instances, the heavy chain CDR1 is selected from SEQ ID NOs:42-48. In certain instances, the heavy chain CDR2 is selected from SEQ ID NOs:49-55. In certain instances, the heavy chain CDR3 is selected from SEQ ID NOs:56-63.

In some embodiments, the light and heavy CDRs are selected without the surrounding framework sequences of the respective variable domains, which include framework sequences from other immunoglobulins or consensus framework regions, optionally are further mutated and/or replaced by other suitable framework sequences. Therefore provided herein in some embodiments, is a CD33 binding domain comprising a light chain variable domain, wherein the light chain CDR1 is SEQ ID NO:21; the light chain CDR2 is SEQ ID NO:28 and the light chain CDR3 is SEQ ID NO:35. In some embodiments, a CD33 binding domain comprises a light chain variable domain, wherein the light chain CDR1 is SEQ ID NO:22; the light chain CDR2 is SEQ ID NO:29 and the light chain CDR3 is SEQ ID NO:36. In some embodiments, a CD33 binding domain comprises a light chain variable domain, wherein the light chain CDR1 is SEQ ID NO:23; the light chain CDR2 is SEQ ID NO:30 and the light chain CDR3 is SEQ ID NO:37. In some embodiments, a CD33 binding domain comprises a light chain variable domain, wherein the light chain CDR1 is SEQ ID NO:24; the light chain CDR2 is SEQ ID NO:31 and the light chain CDR3 is SEQ ID NO:38. In some embodiments, a CD33 binding domain comprises a light chain variable domain, wherein the light chain CDR1 is SEQ ID NO:25; the light chain CDR2 is SEQ ID NO:32 and the light chain CDR3 is SEQ ID NO:39. In some embodiments, a CD33 binding domain comprises a light chain variable domain, wherein the light chain CDR1 is SEQ ID NO:26; the light chain CDR2 is SEQ ID NO:33 and the light chain CDR3 is SEQ ID NO:40. In some embodiments, a CD33 binding domain comprises a light chain variable domain, wherein the light chain CDR1 is SEQ ID NO:27; the light chain CDR2 is SEQ ID NO:34 and the light chain CDR3 is SEQ ID NO:41.

Also provided herein in some embodiments, is a CD33 binding domain comprising a heavy chain variable domain, wherein the heavy chain CDR1 is SEQ ID NO:42; the heavy chain CDR2 is SEQ ID NO:49 and the heavy chain CDR3 is SEQ ID NO:56. In some embodiments, a CD33 binding domain comprises a heavy chain variable domain, wherein the heavy chain CDR1 is SEQ ID NO:43; the heavy chain CDR2 is SEQ ID NO:50 and the heavy chain CDR3 is SEQ ID NO:57. In some embodiments, a CD33 binding domain comprises a heavy chain variable domain, wherein the heavy chain CDR1 is SEQ ID NO:43; the heavy chain CDR2 is SEQ ID NO:50 and the heavy chain CDR3 is SEQ ID NO:58. In some embodiments, a CD33 binding domain comprises a heavy chain variable domain, wherein the heavy chain CDR1 is SEQ ID NO:43; the heavy chain CDR2 is SEQ ID NO:50 and the heavy chain CDR3 is SEQ ID NO:59. In some embodiments, a CD33 binding domain comprises a heavy chain variable domain, wherein the heavy chain CDR1 is SEQ ID NO:43; the heavy chain CDR2 is SEQ ID NO:50 and the heavy chain CDR3 is SEQ ID NO:60. In some embodiments, a CD33 binding domain comprises a heavy chain variable domain, wherein the heavy chain CDR1 is SEQ ID NOA4; the heavy chain CDR2 is SEQ ID NO:51 and the heavy chain CDR3 is SEQ ID NO:61. In some embodiments, a CD33 binding domain comprises a heavy chain variable domain, wherein the heavy chain CDR1 is SEQ ID NO:45; the heavy chain CDR2 is SEQ ID NO:52 and the heavy chain CDR3 is SEQ ID NO:62. In some embodiments, a CD33 binding domain comprises a heavy chain variable domain, wherein the heavy chain CDR1 is SEQ ID NO:46; the heavy chain CDR2 is SEQ ID NO:53 and the heavy chain CDR3 is SEQ ID NO:63. In some embodiments, a CD33 binding domain comprises a heavy chain variable domain, wherein the heavy chain CDR1 is SEQ ID NO:47; the heavy chain CDR2 is SEQ ID NO:54 and the heavy chain CDR3 is SEQ ID NO:63. In some embodiments, a CD33 binding domain comprises a heavy chain variable domain, wherein the heavy chain CDR1 is SEQ ID NO:48; the heavy chain CDR2 is SEQ ID NO:55 and the heavy chain CDR3 is SEQ ID NO:63.

In further embodiments, a CD33 binding domain comprises a variable light chain domain selected from amino acid sequences SEQ ID NOs.:1-10 shown in Table 3. In further embodiments, a CD33 binding domain comprises a variable heavy chain domain selected from amino acid sequences SEQ ID NO:11-20 shown in Table 4. In yet further embodiments, a CD33 binding domain comprises a variable light chain domain selected from amino acid sequences SEQ ID NOs.:1-10 shown in Table 3 and a variable heavy chain domain selected from amino acid sequences SEQ ID NO:11-20 shown in Table 4.

The term “binding domain” refers to an immunoglobulin derivative with antigen binding properties, i.e. immunoglobulin polypeptides or fragments thereof that contain an antigen binding site. The binding domain comprises variable domains of an antibody or fragments thereof. Each antigen-binding domain is formed by an antibody, i.e. immunoglobulin, variable heavy chain domain (VH) and an antibody variable light chain domain (VL) binding to the same epitope, whereas the variable heavy chain domain (VH) comprises three heavy chain complementarity determining regions (CDR): CDR1, CDR2 and CDR3; and the variable light chain domain (VL) comprises three light chain complementarity determining regions (CDR): CDR1, CDR2 and CDR3. In some instances, the binding domain according to some embodiments herein is devoid of immunoglobulin constant domains. In some instances, the variable light and heavy chain domains forming the antigen binding site is covalently linked with one another, e.g. by a peptide linker, or in other instances, the variable light and heavy chain domains non-covalently associate with one another to form the antigen binding site. The term “binding domain” refers also to antibody fragments or antibody derivatives including, for example, Fab, Fab′, F(ab′)₂, Fv fragments, single-chain Fv, tandem single-chain Fv ((scFv)₂, Bi-specific T-cell engagers (BiTE®), dual affinity retargeting antibodies (DART™), diabody, tandem diabody (TandAb®), DuoBody® IgG molecules, 2×2 T-cell engagers, TriTacs, and the like. Furthermore, in certain instances, the binding domain is multivalent, i.e. has two, three or more binding sites for CD33 or CD3.

TABLE 1
Amino acid sequences of anti-CD33 variable light
chain CDR1, CDR2 and CDR3

| | Sequence | |
|---|---|---|
| CDR | identifier | Light Chain CDR Sequence |
| CDR1 | SEQ ID NO: 21 | GGNNIGSTTVH |
| | SEQ ID NO: 22 | SGSRSNIGSNTVN |
| | SEQ ID NO: 23 | SGSSSNIGSNTVN |
| | SEQ ID NO: 24 | TGSSSNIGAGYDVH |
| | SEQ ID NO: 25 | SGSSSNIGSNIVN |
| | SEQ ID NO: 26 | SGSSSNIGSNTVK |
| | SEQ ID NO: 27 | SGSSSNIGDNVVN |
| CDR2 | SEQ ID NO: 28 | DDNERPS |
| | SEQ ID NO: 29 | GNNQRPS |
| | SEQ ID NO: 30 | SDNQRPS |
| | SEQ ID NO: 31 | GNSNRPS |
| | SEQ ID NO: 32 | SNNQRPS |
| | SEQ ID NO: 33 | SNNQRSS |
| | SEQ ID NO: 34 | STNKRPS |
| CDR3 | SEQ ID NO: 35 | QVWDSGSDH |
| | SEQ ID NO: 36 | ATWDDSLIG |
| | SEQ ID NO: 37 | ATWDDSLNG |
| | SEQ ID NO: 38 | QSYDSSLSD |
| | SEQ ID NO: 39 | AAWDDSLKG |
| | SEQ ID NO: 40 | AAWDDSLNG |
| | SEQ ID NO: 41 | AAWDDSLSA |

TABLE 2
Amino acid sequences of anti-CD33 variable heavy
chain CDR1, CDR2 and CDR3

| | Sequence | |
|---|---|---|
| CDR | identifier | Heavy Chain CDR Sequence |
| CDR1 | SEQ ID NO: 42 | SNYGIH |
| | SEQ ID NO: 43 | TSYDIN |
| | SEQ ID NO: 44 | TSYYMH |
| | SEQ ID NO: 45 | TSYWIG |
| | SEQ ID NO: 46 | SSYAIS |
| | SEQ ID NO: 47 | SSYGIS |
| | SEQ ID NO: 48 | DSYAIS |
| CDR2 | SEQ ID NO: 49 | LISYDGNKKFYADSVKG |
| | SEQ ID NO: 50 | WMNPNSGNTGFAQKFQG |
| | SEQ ID NO: 51 | GIINPSGGSTSYAQKFQG |
| | SEQ ID NO: 52 | IIYPGDSDTRYSPSFQG |
| | SEQ ID NO: 53 | GIYPIFGSANYAQKFQG |
| | SEQ ID NO: 54 | GIIPIFGSAHYAQKFQG |
| | SEQ ID NO: 55 | GIIPIFGSAHYSQKFQG |
| CDR3 | SEQ ID NO: 56 | DRLESAAFDY |
| | SEQ ID NO: 57 | DRANTDFSYGMDV |
| | SEQ ID NO: 58 | DRAVTDYYYGMDV |
| | SEQ ID NO: 59 | DRANTDYSFGMDV |
| | SEQ ID NO: 60 | DRANTDYSLGMDV |
| | SEQ ID NO: 61 | DVVPAAIDYYGMDV |
| | SEQ ID NO: 62 | HKRGSDAFDI |
| | SEQ ID NO: 63 | EYYYDSSEWAFDI |

TABLE 3
Amino acid sequences of all anti-CD33 variable light chain domains
(amino acid sequences of variable light chain CDR1, CDR2 and
CDR3 are in bold and underlined)

| anti- | | |
|---|---|---|
| CD33 | Sequence | |
| clone | identifier | Variable light chain (VL) domain Sequence |
| 01 | SEQ ID NO: 1 | SYELTQPPSVSVAPGQTAMITCGGNNIGSTTVHWYQQKPGQAPVLVV |
| | | YDDNERPSGIPERFSGSNSGSTATLTINRVEAGDEADYYCQVWDSGSD |
| | | HVVFGGGTKLTVL |
| 02 | SEQ ID NO: 2 | QSVLTQPPSASGTPGQRVTISCSGSRSNIGSNTVNWYQQLPGTAPKIII |
| | | YGNNQRPSGVPDRFSGSKSGTSASLAISGLQSEDEADYYCATWDDSLI |
| | | GWVFGGGTKLTVL |
| 03 | SEQ ID NO: 3 | QSVLTQPPSASGTPGQRVTISCSGSRSNIGSNTVNWYQQLPGTAPKLLI |
| | | YGNNQRPSGVPDRFSGSKSGTSASLAISGLQSEDEADYYCATWDDSLI |
| | | GWVFGGGTKLTVL |
| 04 | SEQ ID NO: 4 | QSVLTQPPSASGTPGQRVTISCSGSRSNIGSNTVNWYQQLPGTAPKLLI |
| | | YGNNQRPSGVPDRFSGSKSGTSASLAISGLQSEDEADYYCATWDDSLI |
| | | GWVFGGGTKLTVL |
| 05 | SEQ ID NO: 5 | QSVLTQPPSASGTPGQRVTISCSGSRSNIGSNTVNWYQQLPGTAPKLLI |
| | | YGNNQRPSGVPDRFSGSKSGTSASLAISGLQSEDEADYYCATWDDSLI |
| | | GWVFGGGTKLTVL |
| 06 | SEQ ID NO: 6 | QSVLTQPPSASGTPGQRVTISCSGSSSNIGSNTVNWYQQLPGTAPKLLI |
| | | YSDNQRPSGVPDRFSGSKSGSSASLAISGLQSDDEADYYCATWDDSLN |
| | | GAVFGGGTKLTVL |
| 07 | SEQ ID NO: 7 | QSVLTQPPSVSGAPGQRVTISCTGSSSNIGAGYDVHWYQQLPGTAPKL |
| | | LIYGNSNRPSGVPDRFSGSKSGTSASLAITGLQAEDEADYYCQSYDSSL |
| | | SDVVFGGGTKLTVL |
| 08 | SEQ ID NO: 8 | QSVLTQPPSASGTPGQRVTISCSGSSSNIGSNIVNWYQQLPGTAPKLLIY |
| | | SNNQRPSGVPDRFSGSKSGTSASLAISGLQSEDEADYYCAAWDDSLKG |
| | | YVFGGGTKLTVL |
| 09 | SEQ ID NO: 9 | QSVLTQPPSASGTPGQRVTISCSGSSSNIGSNTVKWYQQLPGTAPKLLI |
| | | YSNNQRSSGVPDRFSGSKSGSSASLAISGLQSEDEADYYCAAWDDSLN |
| | | GYVFGGGTKLTVL |
| 10 | SEQ ID NO: 10 | QSVLTQPPSASGTPGQRVTISCSGSSSNIGDNVVNWYQQLPGTAPKLLI |
| | | YSTNKRPSGVPDRFSGSKSGSSASLAISGLQSEDEADYYCAAWDDSLS |
| | | AYVFGGGTKLTVL |

TABLE 4
Amino acid sequence of anti-CD33 variable heavy chain domain
(amino acid sequences of variable heavy chain CDR1, CDR2 and
CDR3 are in bold and underlined)

| anti- | | |
|---|---|---|
| CD33 | Sequence | |
| clone | identifier | Variable heavy chain (VH) domain Sequence |
| 01 | SEQ ID NO: 11 | QVQLQESGGGVVQPGRSLRLSCAASGFSFSNYGIHWVRQAPGKGLEWVA |
| | | LISYDGNKKFYADSVKGRFAISRDTSKNTVDLQMTSLRPEDTAVYYCAK |
| | | DRLESAAFDYWGQGTLVTVSS |
| 02 | SEQ ID NO: 12 | QVQLVQSGAEVKKPGASVKVSCKASGYTFTSYDINWVRQAPGQGLEWM |
| | | GWMNPNSGNTGFAQKFQGRVTMTRDTSTSTVYMELSSLRSEDTAVYYC |
| | | ARDRANTDFSYGMDVWGQGTLVTVSS |
| 03 | SEQ ID NO: 13 | QVQLVQSGAEVKKPGASVKVSCKASGYTFTSYDINWVRQAPGQGLEWM |
| | | GWMNPNSGNTGFAQKFQGRVTMTRDTSTSTVYMELSSLRSEDTAVYYC |
| | | ARDRAVTDYYYGMDVWGQGTLVTVSS |
| 04 | SEQ ID NO: 14 | QVQLVQSGAEVKKPGASVKVSCKASGYTFTSYDINWVRQAPGQGLEWM |
| | | GWMNPNSGNTGFAQKFQGRVTMTRDTSTSTVYMELSSLRSEDTAVYYC |
| | | ARDRANTDYSFGMDVWGQGTLVTVSS |
| 05 | SEQ ID NO: 15 | QVQLVQSGAEVKKPGASVKVSCKASGYTFTSYDINWVRQAPGQGLEWM |
| | | GWMNPNSGNTGFAQKFQGRVTMTRDTSTSTVYMELSSLRSEDTAVYYC |
| | | ARDRANTDYSLGMDVWGQGTLVTVSS |
| 06 | SEQ ID NO: 16 | QVQLVQSGAEVKKPGASVKVSCKASGYTFTSYYMHWVRQAPGQGLEWM |
| | | MGIINPSGGSTSYAQKFQGRVTMTRDTSTSTVYMELSSLRSEDTAVYYC |
| | | ARDVVPAAIDYYGMDVWGQGTTVTVSS |
| 07 | SEQ ID NO: 17 | QVQLVQSGAEVKKPGESLKISCKGSGYSFTSYWIGWVRQMPGKGLEWM |
| | | GIIYPGDSDTRYSPSFQGQVTISADKSISTAYLQWSSLKASDTAMYYCAR |
| | | HKRGSDAFDIWGQGTTVTVSS |
| 08 | SEQ ID NO: 18 | QVQLVQSGAEVKKPGSSVKVSCKASGGTFSSYAISWVRQAPGQGLEWMG |
| | | GIYPIFGSANYAQKFQGRVTITADESTSTAYMELSSLRSEDTAVYYCARE |
| | | YYYDSSEWAFDIWGQGTLVTVSS |
| 09 | SEQ ID NO: 19 | QVQLVQSGAEVKKPGSSVKVSCKASGGTFSSYGISWVRQAPGQGLEWM |
| | | GGIIPIFGSAHYAQKFQGRVTITADESTSTAYMELSSLRSEDTAVYYCAR |
| | | EYYYDSSEWAFDIWGQGTLVTVSS |
| 10 | SEQ ID NO: 20 | QVQLVQSGAEVKKPGSSVKVSCKASGGTFDSYAISWVRQAPGQGLEWM |
| | | GGIIPIFGSAHYSQKFQGRVTITADESTSTAYMELSSLRSEDTAVYYCARE |
| | | YYYDSSEWAFDIWGQGTLVTVSS |

In some embodiments, a binding domain conferring specificity to CD33 is selected from one of the following combinations of a variable heavy chain domain and a variable light chain domain forming the human CD33 binding site shown in Table 3 and in Table 4. Non-limiting examples include (i) SEQ ID NO:1 and SEQ ID NO: 11, (ii) SEQ ID NO:2 and SEQ ID NO: 12, (iii) SEQ ID NO:3 and SEQ ID NO: 13. (iv) SEQ ID NO:4 and SEQ ID NO: 14, (v) SEQ ID NO:5 and SEQ ID NO: 5, (vi) SEQ ID NO:6 and SEQ ID NO: 16, (vii) SEQ ID NO:7 and SEQ ID NO: 17, (viii) SEQ ID NO:8 and SEQ ID NO: 18, (ix) SEQ ID NO:9 and SEQ ID NO: 19, and (x) SEQ ID NO. 10 and SEQ ID NO: 20.

The bispecific antibodies described herein have binding domains that not only have specificity for CD33, but also have at least one further functional domain. In a further embodiment at least one further functional domain is an effector domain. An “effector domain” comprises a binding site of an antibody specific for an effector cell, which can stimulate or trigger cytotoxicity, phagocytosis, antigen presentation, cytokine release. Such effector cells are, for example, but not limited to, T-cells. In particular, the effector domain comprises at least one antibody variable heavy chain domain and at least one variable light chain domain forming an antigen binding site for an antigen on T-cells, such as, for example, human CD3.

Thus, in some embodiments, the bispecific antibody described herein is multifunctional. The term multifunctional as used herein means that a binding protein exhibits two or more different biological functions. For example, the different biological functions are different specificities for different antigens. In certain instances, the multifunctional CD33 binding protein is multispecific, i.e. has binding specificity to CD33 and one or more further antigens. In certain instances, the binding protein is bispecific with specificities for CD33 and CD3 and may be masked or unmasked with other proteins, protein fragments or chemical structures. Such bispecific binding proteins include, for example, bispecific monoclonal antibodies of the classes IgA, IgD, IgE, IgG or IgM, diabodies, single-chain diabodies (scDb), single chain antibodies, nanobodies, tandem single chain Fv (scFv)2, for example Bi-specific T-cell engagers (BiTE®), dual affinity retargeting antibodies (DART™), tandem diabodies (TandAb®), 2×2 T-cell engagers and flexibodies.

CD3, as used herein, denotes an antigen that is expressed on human T cells as part of the multimolecular T cell receptor complex, consisting of five chains: 2 CD3-epsilon, a CD3-gamma, a CD3-delta, and a CD3 zeta. Clustering of CD3 on T cells e.g. by anti-CD3 antibodies leads to T cell activation similar to the binding of an antigen but independent from the clonal specificity of the T cell subset, as described above. Thus, a bispecific antibody specifically binding with one of its specificities the human CD3 antigen relates to a CD3-specific construct capable of binding to the human CD3 complex expressed on human T cells and capable of inducing elimination/lysis of target cells, wherein such target cells carry/display an antigen which is bound by the other, non-CD3-binding portion of the bispecific single chain antibody. Binding of the CD3 complex by CD3-specific binders (e.g. a bispecific single chain antibody as administered according to the pharmaceutical means and methods described herein) leads to activation of T cells.

In certain embodiments, the CD3 binding site of a bispecific antibody to CD33 and CD3 has specificity for human CD3 and, in some instances, cynomolgus CD3. Examples of such a binding site are polypeptides comprising the VH domain CDR1, CDR2 and CDR3 from the sequences shown in Table 5 (SEQ ID NOs:64-67) and VL domain CDR1, CDR2 and CDR3 from the sequence shown in Table 6 (SEQ ID NOs:68-71). In certain instances, a CD3 binding site is the combination of the variable heavy chain domain of SEQ ID NO:64 and the variable light chain domain of SEQ ID NO:68. In certain instances, a CD3 binding site is the combination of the variable heavy chain domain of SEQ ID NO:65 and the variable light chain domain of SEQ ID NO:69. In certain instances, a CD3 binding site is the combination of the variable heavy chain domain of SEQ ID NO:66 and the variable light chain domain of SEQ ID NO:70. In certain instances, a CD3 binding site is the combination of the variable heavy chain domain of SEQ ID NO:67 and the variable light chain domain of SEQ ID NO:71.

TABLE 5
Amino acid sequence of an anti-CD3 variable heavy chain domain
(amino acid sequences of variable heavy chain CDR1, CDR2 and
CDR3 are in bold and underlined)

| anti-CD3 | VH domain Sequence |
|---|---|
| SEQ ID NO: 64 | EVQLVNESGGGLVQPGGSLRLSCAASGFTFSTYAMNWVRQAPGKGLEWVGRIRSKYNNY |
| CD3-01 | ATYYADSVKDRFTISRDDSKNSLYLQMNSLKTEDTAVYYCARHGNFGNSYVSYFAYWG |
| | QGTLVTVSS |
| SEQ ID NO: 65 | EVQLVESGGGLVQPGGSLRLSCAASGFTFSTYAMNWVRQAPGKGLEWVGRIRSKYNNY |
| CD3-02 | ATYYADSVKDRFTISRDDSKNSLYLQMNSLKTEDTAVYYCARHGNFGNSYVSWFAYWG |
| | QGTLVTVSS |
| SEQ ID NO: 66 | EVQLVESGGGLVQPGGSLRLSCAASGFTFSTYAMNWVRQAPGKGLEWVGRIRSKYNNY |
| CD3-03 | ATYYADSVKDRFTISRDDSKNSLYLQMNSLKTEDTAVYYCARHGNFGNSYVSWFAYWG |
| | QGTLVTVSS |
| SEQ ID NO: 67 | EVQLVESGGGLVQPGGSLRLSCAASGFTFSTYAMNWVRQAPGKGLEWVGRIRSKYNNY |
| CD3-04 | ATYYADSVKDRFTISRDDSKNSLYLQMNSLKTEDTAVYYCARHGNFGNSYVSWFAYWG |
| | QGTLVTVSS |

TABLE 6
Amino acid sequence of an anti-CD3 variable light chain domain
(amino acid sequences of variable light chain CDR1, CDR2 and
CDR3 are in bold and underlined)

| anti-CD3 | VL domain Sequence |
|---|---|
| SEQ ID NO: 68 | DIQMTQSPSSLSASVGDRVTITCRSSTGAVTTSNYANWVQQKPGKAPKALIGGTNKRAP |
| CD3-01 | GVPSRFSGSLIGDKATLTISSLQPEDFATYYCALWYSNLWVFGQGTKVEIK |
| SEQ ID NO: 69 | DIQMTQSPSSLSASVGDRVTITCRSSTGAVTTSNYANWVQQKPGKAPKGLIGGTNKRAP |
| CD3-02 | GVPARFSGSGSGTDFTLTISSLQPEDFATYYCALWYSNLWVFGQGTKVEIK |
| SEQ ID NO: 70 | DIQMTQSPSSLSASVGDRVTITCRSSTGAVTTSNYANWVQQKPGKAPKGLIGGTNKRAP |
| CD3-03 | GVPSRFSGSLIGDKATLTISSLQPEDFATYYCALWYSNLWVFGQGTKVEIK |
| SEQ ID NO: 71 | DIQMTQSPSSLSASVGDRVTITCRSSTGAVTTSNYANWVQQKPGKAPKGLIGGTNKRAP |
| CD3-04 | GVPSRFSGSLIGTDFTLTISSLQPEDFATYYCALWYSNLWVFGQGTKVEIK |

In further embodiments, the CD3 binding site of a bispecific antibody to CD33 and CD3 has a variable heavy chain domain comprising a CDR1 sequence of STYAMN (SEQ ID NO:72). In further embodiments, the CD3 binding site of a bispecific antibody to CD33 and CD3 has a variable heavy chain domain comprising a CDR2 sequence of RIRSKYNNYATYYADSVKD (SEQ ID NO:73). In further embodiments, the CD3 binding site of a bispecific antibody to CD33 and CD3 has a variable heavy chain domain comprising a CDR3 sequence of HGNFGNSYVSWFAY (SEQ ID NO:74). In further embodiments, the CD3 binding site of a bispecific antibody to CD33 and CD3 has a variable heavy chain domain comprising a CDR3 sequence of HGNFGNSYVSYFAY (SEQ ID NO:75). In yet further embodiments, the CD3 binding site has a variable heavy chain domain comprising a CDR1, CDR2 and CDR3 sequence of SEQ ID NOs:72-74 respectively. In yet further embodiments, the CD3 binding site has a variable heavy chain domain comprising a CDR1, CDR2 and CDR3 sequence of SEQ ID NOs: 72, 73 and 75 respectively.

In further embodiments, the CD3 binding site of a bispecific antibody to CD33 and CD3 has a variable heavy chain domain comprising a CDR1 sequence selected from the group consisting of NTYAMN (SEQ ID NO:76), NTYAMH (SEQ ID NO:77) and NKYAMN (SEQ ID NO:78). In further embodiments, the CD3 binding site of a bispecific antibody to CD33 and CD3 has a variable heavy chain domain comprising a CDR2 sequence selected from the group consisting of RIRNKYNNYATYYADSVKD (SEQ ID NO:79), RIRNKYNNYATEYADSVKD (SEQ ID NO:80), RIRSKYNNYATEYAASVKD (SEQ ID NO:81), RIRNKYNNYATEYAASVKD (SEQ ID NO:82), RIRSKYNNYATYYADSVKG (SEQ ID NO:83) and RIRSKYNNYATEYADSVKS (SEQ ID NO:84). In further embodiments, the CD3 binding site of a bispecific antibody to CD33 and CD3 has a variable heavy chain domain comprising a CDR3 sequence selected from the group consisting of HGNFGDSYVSWFAY (SEQ ID NO:85), HGNFGNTYVSWFAY (SEQ ID NO:86), HGNFGCSYVSWFAY (SEQ ID NO:87), HGNFGNSYISYWAY (SEQ ID NO:88) and HGNFGNSYVSFFAY (SEQ ID NO:89).

In yet further embodiments, the CD3 binding site has a variable heavy chain domain comprising a CDR1, CDR2 and CDR3 sequence of SEQ ID NOs:76, 73 and 74 respectively, SEQ ID NOs:76, 79 and 74 respectively, SEQ ID NOs:76, 80 and 74 respectively. SEQ ID NOs:76, 81 and 74 respectively, SEQ ID NOs:76, 82 and 74 respectively, SEQ ID NOs:76, 83 and 74 respectively, SEQ ID NOs:72, 83 and 74 respectively, SEQ ID NOs:72, 83 and 85 respectively, SEQ ID NOs:76, 83 and 86 respectively, SEQ ID NOs:77, 83 and 74 respectively, SEQ ID NOs:72, 83 and 87 respectively, SEQ ID NOs:78, 73 and 88 respectively or SEQ ID NOs:78, 84 and 89 respectively.

In further embodiments, the CD3 binding site of a bispecific antibody to CD33 and CD3 has a variable light chain domain comprising a CDR1 sequence of RSSTGAVTTSNYAN (SEQ ID NO:90). In further embodiments, the CD3 binding site of a bispecific antibody to CD33 and CD3 has a variable light chain domain comprising a CDR2 sequence of GTNKRAP (SEQ ID NO:91). In further embodiments, the CD3 binding site of a bispecific antibody to CD33 and CD3 has a variable light chain domain comprising a CDR3 sequence of ALWYSNL (SEQ ID NO:92). In yet further embodiments, the CD3 binding site has a variable light chain domain comprising a CDR1, CD2 and CD3 sequence of SEQ ID NOs:90-92 respectively.

In certain instances, the CD3 binding site has a high affinity to CD3. Alternatively, in other instances, the CDR1, CDR2, CDR3 from the heavy-chain domain as well as the light-chain domain or, optionally, the variable light-chain domains and variable heavy-chain domains is derived from other CD3 antibodies, such as, for example UCHT1, muromonab-CD3 (OKT3), otelixizumab (TRX4), teplizumab (MGA031), visilizumab (Nuvion), and the like.

In another aspect, described herein are bispecific antibodies to CD33 and CD3 that are humanized or fully human, i.e. of human origin. In further embodiments, described herein are bispecific antibodies to CD33 and CD3 that are camelid or llama.

In some embodiments, a bispecific antibody to CD33 and CD3 has one of the following combinations providing CD33 and CD3 specificity by variable light and heavy chain domains for CD33 and CD3: include, but are not limited to, (i) SEQ ID NOs:2, 12, 65 and 69, (ii) SEQ ID NOs:3, 13, 65 and 69, (iii) SEQ ID NOs:4, 14, 65 and 69, (iv) SEQ ID NOs:5, 15, 65 and 69, (v) SEQ ID NOs:1, 11, 64 and 68, (vi) SEQ ID NOs:2, 12, 64 and 68, (vii) SEQ ID NOs:2, 12, 66 and 70, (viii) SEQ ID NOs:4, 14, 66 and 70, (ix) SEQ ID NOs:5, 15, 66 and 70, and (x) SEQ ID NOs:3, 13, 64 and 68, (xi) SEQ ID NOs:3, 13, 67 and 71, (xii) SEQ ID NOs:4, 14, 64 and 68, (xiii) SEQ ID NOs:5, 15, 64 and 68, (xiv) SEQ ID NOs:7, 17, 64 and 68, (xv) SEQ ID NOs:6, 16, 64 and 68, (xvi) SEQ ID NOs:6, 16, 67 and 71, (xvii) SEQ ID NOs:8, 18, 64 and 68, (xviii) SEQ ID NOs:9, 19, 64 and 68. (xix) SEQ ID NOs:9, 19, 67 and 71, and (xx) SEQ ID NOs:10, 20, 64 and 68.

### Conserved Variants of CDR Sequences and Heavy and Light Chain Domains

In alternative embodiments, the heavy and light chain domains incorporate immunologically active homologues or variants of the CDR sequences described herein. Accordingly in some embodiments, a CDR sequence in a heavy or light chain domain that binds to CD33 or CD3 is similar to, but not identical to, the amino acid sequence depicted in SEQ ID NOs: 21-63 or 72-92. In certain instances, a CDR variant sequence has a sequence identity of 99%, 98%, 97%, 96%, 95%, 94%, 93%, 92%, 91%, 900%, 89%, 88%, 87%, 86%, 85%, 84%, 83%, 82%, 81%, or 80% compared to the sequence of SEQ ID NOs: 21-63 or 72-90 and which is immunologically active.

In further instances, a CDR variant sequence incorporates 1, 2, 3, 4, or 5 conserved amino acid substitutions. Conservative substitutions include amino acid substitutions that substitute a given amino acid with another amino acid of similar characteristics and further include, among the aliphatic amino acids interchange of alanine, valine, leucine, and isoleucine; interchange of the hydroxyl residues serine and threonine, exchange of the acidic residues aspartate and glutamate, substitution between the amide residues asparagine and glutamine, exchange of the basic residues lysine and arginine, and replacements among the aromatic residues phenylalanine and tyrosine.

In yet further instances, a CDR variant sequence incorporates substitutions that enhance properties of the CDR such as increase in stability, resistance to proteases and/or binding affinities to CD33 or CD3.

In other instances, a CDR variant sequence is modified to change non-critical residues or residues in non-critical regions. Amino acids that are not critical can be identified by known methods, such as affinity maturation, CDR walking, site-directed mutagenesis, crystallization, nuclear magnetic resonance, photoaffinity labeling, or alanine-scanning mutagenesis.

In further alternative embodiments, the bispecific antibodies to CD33 and CD3 comprise heavy and light chain domains that are immunologically active homologues or variants of heavy and light chain domain sequences provided herein. Accordingly, in some embodiments, a CD33 and CD3 binding protein comprises a heavy or light chain domain sequence that is similar to, but not identical to, the amino acid sequence depicted in SEQ ID NOs:1-20 or 64-71. In certain instances, a variant heavy or light chain domain sequence has a sequence identity of 99%, 98%, 97%, 96%, 95%, 94%, 93%, 92%, 91%, 900%, 89%, 88%, 87%, 86%, 85%, 84%, 83%, 82%, 81%, or 80% compared to the sequence of SEQ ID NOs:1-20 or 64-71 and which is immunologically active.

In further instances, a variant heavy or light chain domain sequence incorporates 1, 2, 3, 4, or 5 conserved amino acid substitutions. Conservative substitutions include amino acid substitutions that substitute a given amino acid with another amino acid of similar characteristics and further include, among the aliphatic amino acids interchange of alanine, valine, leucine, and isoleucine; interchange of the hydroxyl residues serine and threonine, exchange of the acidic residues aspartate and glutamate, substitution between the amide residues asparagine and glutamine, exchange of the basic residues lysine and arginine, and replacements among the aromatic residues phenylalanine and tyrosine.

In yet further instances, a variant heavy or light chain domain sequence incorporates substitutions that enhance properties of the CDR such as increase in stability, resistance to proteases and/or binding affinities to CD33 or CD3.

In other instances, a variant heavy or light chain domain sequence is modified to change non-critical residues or residues in non-critical regions. Amino acids that are not critical can be identified by known methods, such as affinity maturation, CDR walking, site-directed mutagenesis, crystallization, nuclear magnetic resonance, photoaffinity labeling, or alanine-scanning mutagenesis.

### CD33 and CD3 2×2 T-Cell Engagers

In another aspect, a bispecific antibody to CD33 and CD3 is a dimer, i.e. comprises two polypeptides with antigen binding sites for CD33 and CD3.

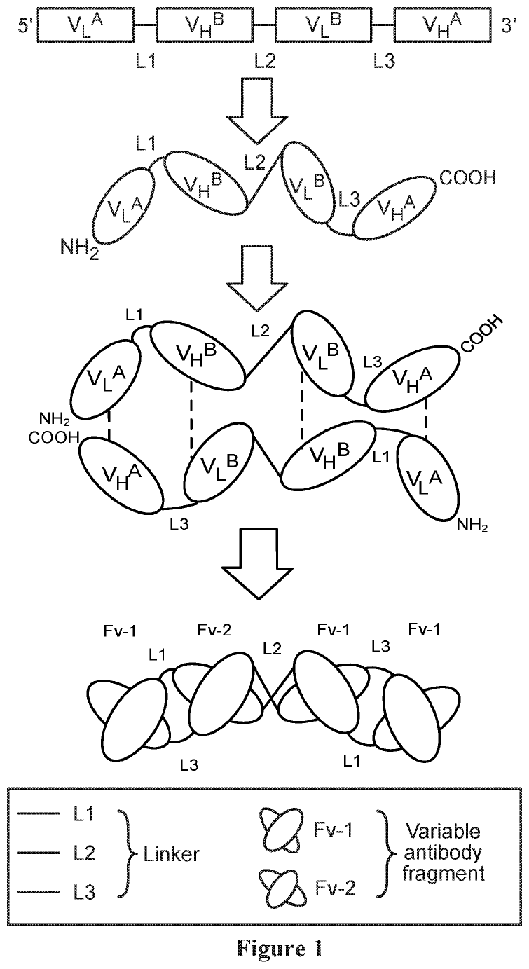

Also provided herein in another aspect, is a dimeric and bispecific antibody to CD33 and CD3 in the format of a 2×2 T-cell engager. Such 2×2 T-cell engagers are constructed by linking four antibody variable binding domains (two heavy-chain variable domains (VH) and two light-chain variable domains (VL) in a single gene construct (FIG. 1) enabling homo-dimerization. In such 2×2 T-cell engagers the linker length is such that it prevents intramolecular pairing of the variable domains so that the molecule cannot fold back upon itself to form a single-chain diabody, but rather is forced to pair with the complementary domains of another chain. The domains are also arranged such that the corresponding VH and VL domains pair during this dimerization. Following expression from a single gene construct, two identical polypeptide chains fold head-to-tail forming a functional non-covalent homodimer of approximately 105 kDa (FIG. 1). Despite the absence of intermolecular covalent bonds, the homodimer is highly stable once formed, remains intact and does not revert back to the monomeric form.

2×2 T-cell engagers have a number of properties that provide advantages over traditional monoclonal antibodies and other smaller bispecific molecules. 2×2 T-cell engagers contain only antibody variable domains and therefore are contemplated to lack side effects or non-specific interactions that may be associated with an Fc moiety. For example, Fc receptors which can bind to Fc domains are found on numerous cell types such as white blood cells (e.g., basophils, B-cells, eosinophils, natural killer cells, neutrophils and the like) or Kuppfer cells. Because 2×2 T-cell engagers allow for bivalent binding to each of CD33 and CD3, the molecules have avidity that is similar to that of an IgG antibody against a single target. The size of a 2×2 T-cell engager, at approximately 105 kDa, is smaller than that of an IgG, which may allow for enhanced tumor penetration. However, this size is well above the renal threshold for first-pass clearance, offering a pharmacokinetic advantage compared with smaller bispecific formats based on antibody-binding domains or non-antibody scaffolds. Moreover 2-2 T-cell engagers are advantageous over other bispecific binding proteins such as BiTE or DART molecules based on these pharmacokinetic and avidity properties resulting in longer intrinsic half-lives and rapid cytotoxicity. 2×2 T-cell engagers are well expressed in host cells, for example, mammalian CHO cells. It is contemplated that robust upstream and downstream manufacturing processes are available for 2×2 T-cell engagers.

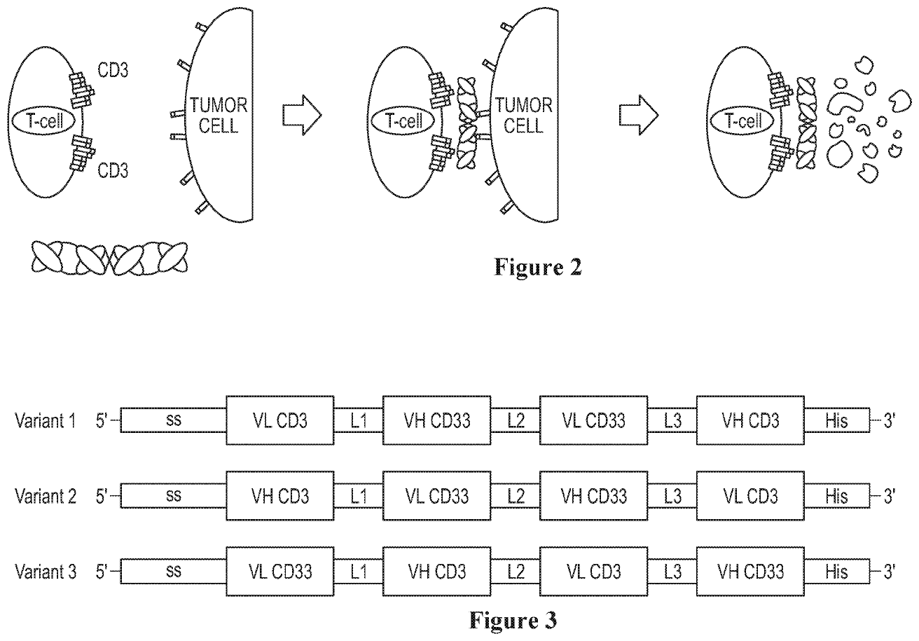

The CD33 and CD3 bispecific 2×2 T-cell engagers described herein are designed to allow specific targeting of tumor cells and cells in the tumor microenvironment, such as MDSCs, that express CD33 by recruiting cytotoxic T-cells. In contrast, by engaging CD3 molecules expressed specifically on these cells, the 2×2 T-cell engager can bind cytotoxic T-cells and CD33 expressing cells in a highly specific fashion, thereby significantly increasing the cytotoxic potential of such molecules. This mechanism is outlined in FIG. 2. It is reported that T-cells can play a role in controlling tumor growth. For example, the presence of cytotoxic T-cells in colorectal tumors as well as lymph nodes from NHL patients was shown to correlate with a better clinical outcome. Furthermore, the potential of therapies designed to induce T-cell responses has been demonstrated for melanoma vaccines, as well as the antibody directed against CTLA-4, a negative regulator of T-cell activation. The 2×2 T-cell engagers described herein engage cytotoxic T-cells via binding to the surface-expressed CD3, which forms part of the T-cell receptor. Simultaneous binding of this 2×2 T-cell engager to CD3 and to CD33 expressed on the surface of particular tumor cells causes T-cell activation and mediates the subsequent lysis of the malignant cell (FIG. 2).

Therefore, in a further aspect is a multispecific, 2×2 T-cell engager. In some embodiments, a multispecific 2-2 T-cell engager has specificities to two, three or more different epitopes, wherein two or more epitopes can be of the same antigen target or of different antigen targets. In certain embodiments the multispecific, 2×2 T-cell engager is bispecific and tetravalent, i.e. comprises four antigen-binding sites. Such a bispecific 2-2 T-cell engager binds with at least one antigen-binding site, to human CD3 and to human CD33, wherein in certain instances, the 2-2 T-cell engager binds with two antigen-binding sites to human CD3 and with two other antigen-binding sites to human CD33, i.e. the 2×2 T-cell engager binds bivalently to each antigen.

In some embodiments, a bispecific, antigen-binding 2×2 T-cell engager is specific to human CD33 and human CD3, wherein said 2×2 T-cell engager comprises a first polypeptide and a second polypeptide, each polypeptide having at least four variable chain domains linked one after another, wherein each polypeptide comprises
(i) a variable heavy chain (VH) domain specific to human CD33;
(ii) a variable light chain (VL) domain specific to human CD33;
(iii) a VH domain specific for human CD3, and
(iv) a VL domain specific for human CD3.

In particular embodiments, a bispecific 2×2 T-cell engager specifically binds to an epitope of human CD33 which is within ₆₂DQEVQEETQ₇₀ (SEQ ID NO:94) (amino acid residues 62-70 of SEQ ID NO:93) of human CD33. In particular instances, such a 2×2 T-cell engager comprises a first polypeptide and a second polypeptide, each polypeptide having at least four variable chain domains linked one after another, wherein each polypeptide comprises
(i) a variable heavy chain domain specific to an epitope of human CD33 which is within ₆₂DQEVQEETQ₇₀ (SEQ ID NO:94) (amino acid residues 62-70 of SEQ ID NO:93) of human CD33;
(ii) a variable light chain domain specific to an epitope of human CD33 which is within ₆₂DQEVQEETQ₇₀ (SEQ ID NO:94) (amino acid residues 62-70 of SEQ ID NO:93) of human CD33;
(iii) a variable heavy chain domain specific for human CD3, and
(iv) a variable light chain domain specific for human CD3.

In other embodiments, described herein are CD33/CD3 2×2 T-cell engagers that have an affinity to CD33 on CD33⁺ cells with a K_{D} of 10 nM or less, 5 nM or less, 1 nM or less, or 0.5 nM or less. The CD33⁺ cells can be selected from tumor cells such as, for example, HL-60 or KG-1.

In a further embodiment a CD33/CD3 2×2 T-cell engager described herein binds CD3 and in certain instances, the epsilon chain of CD3 on CD3⁺ cells, particularly T-cells, with a K_{D} of 10 nM or less, 5 nM or less or 2 nM or less.

In some embodiments, each polypeptide of a bispecific 2×2 T-cell engager comprises one of the following combinations of the four variable chain domains: (i) SEQ ID NOs:2, 12, 65 and 69, (ii) SEQ ID NOs:3, 13, 65 and 69, (iii) SEQ ID NOs:4, 14, 65 and 69, (iv) SEQ ID NOs:5, 15, 65 and 69, (v) SEQ ID NOs:1, 11, 64 and 68, (vi) SEQ ID NOs:2, 12, 64 and 68, (vii) SEQ ID NOs:2, 12, 66 and 70, (viii) SEQ ID NOs:4, 14, 66 and 70, (ix) SEQ ID NOs:5, 15, 66 and 70, and (x) SEQ ID NOs:3, 13, 64 and 68, (xi) SEQ ID NOs:3, 13, 67 and 71, (xii) SEQ ID NOs:4, 14, 64 and 68, (xiii) SEQ ID NOs:5, 15, 64 and 68, (xiv) SEQ ID NOs:7, 17, 64 and 68, (xv) SEQ ID NOs:6, 16, 64 and 68, (xvi) SEQ ID NOs:6, 16, 67 and 71, (xvii) SEQ ID NOs:8, 18, 64 and 68, (xviii) SEQ ID NOs:9, 19, 64 and 68; (xix) SEQ ID NOs:9, 19, 67 and 71, and (xx) SEQ ID NOs:10, 20, 64 and 68.

As used herein, “dimer” refers to a complex of two polypeptides. In certain embodiments, the two polypeptides are non-covalently associated with each other, in particular with the proviso that there is no covalent bond between the two polypeptides. In certain instances, the two polypeptides have covalent associations such as disulfide bonds that form to aid in stabilization of the dimer. In certain embodiments, the dimer is homodimeric, i.e. comprises two identical polypeptides. The term “polypeptide” refers to a polymer of amino acid residues linked by amide bonds. The polypeptide is, in certain instances, a single chain fusion protein, which is not branched. In the polypeptide the variable antibody domains are linked one after another. The polypeptide, in other instances, may have contiguous amino acid residues in addition to the variable domain N-terminal and/or C-terminal residues. For example, such contiguous amino acid residues may comprise a Tag sequence, in some instances at the C-terminus, which is contemplated to be useful for the purification and detection of the polypeptide.

In one aspect, each polypeptide of the bispecific 2×2 T-cell engager comprises four variable domains, a variable light chain (VL) and a variable heavy chain (VH) of a CD3 binding protein as well as a variable light chain (VL) and a variable heavy chain (VH) of a CD33 binding protein. In certain embodiments, four variable domains are linked by peptide linkers L1, L2 and L3 and in some instances arranged from the N- to the C-terminus as follows:

### Domain Order:

### (1) VL(CD3)-L1-VH(CD33)-L2-VL(CD33)-L3-VH(CD3); or

### (2) VH(CD3)-L1-VL(CD33)-L2-VH(CD33)-L3-VL(CD3); or

### (3) VL(CD33)-L1-VH(CD3)-L2-VL(CD3)-L3-VH(CD33); or

### (4) VH(CD33)-L1-VL(CD3)-L2-VH(CD3)-L3-VL(CD33).

The length of the linkers influences the flexibility of the antigen-binding 2×2 T-cell engager according to reported studies. Accordingly, in some embodiments, the length of the peptide linkers L1, L2 and L3 is such that the domains of one polypeptide can associate intermolecularly with the domains of another polypeptide to form the dimeric antigen-binding 2×2 T-cell engager. In certain embodiments, such linkers are “short”, i.e. consist of 0, 1, 2, 3, 4, 5, 6, 7, 8, 9, 10, 11 or 12 amino acid residues. Thus, in certain instances, the linkers consist of about 12 or less amino acid residues. In the case of 0 amino acid residues, the linker is a peptide bond. Such short linkers favor the intermolecular dimerization of the two polypeptides by binding and forming correct antigen-binding sites between antibody variable light chain domains and antibody variable heavy chain domains of different polypeptides. Shortening the linker to about 12 or less amino acid residues generally prevents adjacent domains of the same polypeptide chain from intramolecular interaction with each other. In some embodiments, these linkers consist of about 3 to about 10, for example 4, 5 or 6 contiguous amino acid residues.

Regarding the amino acid composition of the linkers, peptides are selected that do not interfere with the dimerization of the two polypeptides. For example, linkers comprising glycine and serine residues generally provide protease resistance. The amino acid sequence of the linkers can be optimized, for example, by phage-display methods to improve the antigen binding and production yield of the antigen-binding polypeptide dimer. Examples of peptide linkers suitable for a 2×2 T-cell engager in some embodiments are GGSGGS (SEQ ID NO:95), GGSG (SEQ ID NO:96), or GGSGG (SEQ ID NO:97).

Non-limiting examples of 2×2 T-cell engagers as described herein are 2×2 T-cell engagers having an anti-CD33 VL and VH domain, an anti-CD3 VL and VH domain, domain order and linker according to Table 7.

TABLE 7
Exemplary CD33/CD3 2 × 2 T-cell engagers

| 2 × 2 | | | | | | | |
|---|---|---|---|---|---|---|---|
| T-cell | Anti-CD33 domain | | Anti-CD3 domain | | Domain | Linker | |
| engager | VL | VH | VH | VL | Order | L1/L3 | L2 |
| 01 | SEQ ID | SEQ ID | SEQ ID | SEQ ID | 1 | GGSGGS | GGSG |
| | NO: 2 | NO: 12 | NO: 65 | NO: 69 | | | |
| 02 | SEQ ID | SEQ ID | SEQ ID | SEQ ID | 1 | GGSGGS | GGSG |
| | NO: 3 | NO: 13 | NO: 65 | NO: 69 | | | |
| 03 | SEQ ID | SEQ ID | SEQ ID | SEQ ID | 1 | GGSGGS | GGSG |
| | NO: 4 | NO: 14 | NO: 65 | NO: 69 | | | |
| 04 | SEQ ID | SEQ ID | SEQ ID | SEQ ID | 1 | GGSGGS | GGSG |
| | NO: 5 | NO: 15 | NO: 65 | NO: 69 | | | |
| 05 | SEQ ID | SEQ ID | SEQ ID | SEQ ID | 1 | GGSGGS | GGSGG |
| | NO: 4 | NO: 14 | NO: 65 | NO: 69 | | | |
| 06 | SEQ ID | SEQ ID | SEQ ID | SEQ ID | 1 | GGSGGS | GGSGG |
| | NO: 5 | NO: 15 | NO: 65 | NO: 69 | | | |
| 07 | SEQ ID | SEQ ID | SEQ ID | SEQ ID | 1 | GGSGGS | GGSGGS |
| | NO: 1 | NO: 11 | NO: 64 | NO: 68 | | | |
| 08 | SEQ ID | SEQ ID | SEQ ID | SEQ ID | 3 | GGSGGS | GGSGGS |
| | NO: 2 | NO: 12 | NO: 64 | NO: 68 | | | |
| 09 | SEQ ID | SEQ ID | SEQ ID | SEQ ID | 1 | GGSGGS | GGSG |
| | NO: 2 | NO: 12 | NO: 66 | NO: 70 | | | |
| 10 | SEQ ID | SEQ ID | SEQ ID | SEQ ID | 1 | GGSGGS | GGSG |
| | NO: 4 | NO: 14 | NO: 66 | NO: 70 | | | |
| 11 | SEQ ID | SEQ ID | SEQ ID | SEQ ID | 1 | GGSGGS | GGSG |
| | NO: 5 | NO: 15 | NO: 66 | NO: 70 | | | |
| 12 | SEQ ID | SEQ ID | SEQ ID | SEQ ID | 1 | GGSGGS | GGSG |
| | NO: 3 | NO: 13 | NO: 64 | NO: 68 | | | |
| 13 | SEQ ID | SEQ ID | SEQ ID | SEQ ID | 1 | GGSGGS | GGSG |
| | NO: 3 | NO: 13 | NO: 67 | NO: 71 | | | |
| 14 | SEQ ID | SEQ ID | SEQ ID | SEQ ID | 1 | GGSGGS | GGSG |
| | NO: 2 | NO: 12 | NO: 64 | NO: 68 | | | |
| 15 | SEQ ID | SEQ ID | SEQ ID | SEQ ID | 1 | GGSGGS | GGSG |
| | NO: 4 | NO: 14 | NO: 64 | NO: 68 | | | |
| 16 | SEQ ID | SEQ ID | SEQ ID | SEQ ID | 1 | GGSGGS | GGSG |
| | NO: 5 | NO: 15 | NO: 64 | NO: 68 | | | |
| 17 | SEQ ID | SEQ ID | SEQ ID | SEQ ID | 1 | GGSGGS | GGSG |
| | NO: 7 | NO: 17 | NO: 64 | NO: 68 | | | |
| 18 | SEQ ID | SEQ ID | SEQ ID | SEQ ID | 2 | GGSGGS | GGSG |
| | NO: 7 | NO: 17 | NO: 64 | NO: 68 | | | |
| 19 | SEQ ID | SEQ ID | SEQ ID | SEQ ID | 1 | GGSGGS | GGSG |
| | NO: 6 | NO: 16 | NO: 64 | NO: 68 | | | |
| 20 | SEQ ID | SEQ ID | SEQ ID | SEQ ID | 1 | GGSGGS | GGSG |
| | NO: 6 | NO: 16 | NO: 67 | NO: 71 | | | |
| 21 | SEQ ID | SEQ ID | SEQ ID | SEQ ID | 1 | GGSGGS | GGSG |
| | NO: 8 | NO: 18 | NO: 64 | NO: 68 | | | |
| 22 | SEQ ID | SEQ ID | SEQ ID | SEQ ID | 1 | GGSGGS | GGSG |
| | NO: 9 | NO: 19 | NO: 64 | NO: 68 | | | |
| 23 | SEQ ID | SEQ ID | SEQ ID | SEQ ID | 1 | GGSGGS | GGSG |
| | NO: 9 | NO: 19 | NO: 67 | NO: 71 | | | |
| 24 | SEQ ID | SEQ ID | SEQ ID | SEQ ID | 1 | GGSGGS | GGSG |
| | NO: 10 | NO: 20 | NO: 64 | NO: 68 | | | |

In some embodiments, a 2×2 T-cell engager is attached to a C-terminal hexa-histidine (6×His)-tag. In some embodiments, a 2×2 T-cell engager with a C-terminal hexa-histidine (6×His)-tag is 2×2 T-cell engager 01 (SEQ ID NO:98), 02 (SEQ ID NO:99), 03 (SEQ ID NO: 100), 04 (SEQ ID NO:101), 05 (SEQ ID NO: 102), 06 (SEQ ID NO: 103), 07 (SEQ ID NO: 104), 08 (SEQ ID NO: 105), 09 (SEQ ID NO: 106), 10 (SEQ ID NO: 107), 11 (SEQ ID NO: 108), 12 (SEQ ID NO: 109), 13 (SEQ ID NO: 110), 14 (SEQ ID NO:111), 15 (SEQ ID NO:112), 16 (SEQ ID NO:113), 17 (SEQ ID NO: 114), 18 (SEQ ID NO: 115), 19 (SEQ ID NO: 116), 20 (SEQ ID NO:117), 21 (SEQ ID NO:118), 22 (SEQ ID NO:119), 23 (SEQ ID NO: 120), or 24 (SEQ ID NO:121) as depicted in FIGS. 10A to 10X.

In some embodiments, a 2×2 T-cell engager is 2×2 T-cell engager 01 (SEQ ID NO: 123), 02 (SEQ ID NO: 124), 03 (SEQ ID NO: 125), 04 (SEQ ID NO: 126), 05 (SEQ ID NO. 127), 06 (SEQ ID NO: 128), 07 (SEQ ID NO: 129), 08 (SEQ ID NO: 130), 09 (SEQ ID NO:131), 10 (SEQ ID NO: 132), 11 (SEQ ID NO:133), 12 (SEQ ID NO:134), 13 (SEQ ID NO:135), 14 (SEQ ID NO:136), 15 (SEQ ID NO:137), 16 (SEQ ID NO:138), 17 (SEQ ID NO:139), 18 (SEQ ID NO:140), 19 (SEQ ID NO:141), 20 (SEQ ID NO:142), 21 (SEQ ID NO: 143), 22 (SEQ ID NO: 144), 23 (SEQ ID NO: 145), or 24 (SEQ ID NO: 146) as depicted in FIG. 11A to 11X.

The bispecific antibody to CD33 and CD3 (e.g., CD33/CD3 bispecific 2×2 T-cell engager) described herein is produced, in some embodiments, by expressing polynucleotides encoding the polypeptide of the 2×2 T-cell engager which associates with another identical polypeptide to form the antigen-binding 2×2 T-cell engager. Therefore, another aspect is a polynucleotide, e.g. DNA or RNA, encoding the polypeptide of an antigen-binding 2×2 T-cell engager as described herein.

The polynucleotide is constructed by known methods such as by combining the genes encoding at least four antibody variable domains either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. Depending on the vector system and host utilized, any number of suitable transcription and translation elements, including constitutive and inducible promoters, may be used. The promoter is selected such that it drives the expression of the polynucleotide in the respective host cell.

In some embodiments, the polynucleotide is inserted into a vector, preferably an expression vector, which represents a further embodiment. This recombinant vector can be constructed according to known methods.

A variety of expression vector/host systems may be utilized to contain and express the polynucleotide encoding the polypeptide of the described antigen-binding 2×2 T-cell engager. Examples of expression vectors for expression in E. coli are pSKK (Le Gall et al., J Immunol Methods. (2004) 285(1):111-27) or pcDNA5 (Invitrogen) for expression in mammalian cells.

Thus, the antigen-binding 2×2 T-cell engager as described herein, in some embodiments, is produced by introducing a vector encoding the polypeptide as described above into a host cell and culturing said host cell under conditions whereby the polypeptide chains are expressed, may be isolated and, optionally, further purified.

In other aspects, the bispecific antibody to CD33 and CD3 (e.g., CD33/CD3 bispecific 2×2 T-cell engager) described herein has a modification. Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, drug conjugation, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. In further embodiments, the bispecific antibody to CD33 and CD3 is modified with additional amino acids, such as a leader or secretory sequence or a sequence for purification of the polypeptide.

In further aspects, the bispecific antibody to CD33 and CD3 (e.g., CD33/CD3 bispecific 2×2 T-cell engager) described herein comprises a half-life extension domain that extends half-life of the bispecific antibody. Such domains are contemplated to include but are not limited to HSA binding domains, pegylation, small molecules, and other half-life extension domains known in the art. Human serum albumin (HSA) (molecular mass ˜67 kDa) is the most abundant protein in plasma, present at about 50 mg/ml (600 μM), and has a half-life of around 20 days in humans. HSA serves to maintain plasma pH, contributes to colloidal blood pressure, functions as carrier of many metabolites and fatty acids, and serves as a major drug transport protein in plasma. Noncovalent association with albumin extends the elimination half-time of proteins. In some embodiments, the half-life extension domain is a domain that binds to HSA including but not limited to domains from a monoclonal antibody, a polyclonal antibody, a recombinant antibody, a human antibody, a humanized antibody, a single chain variable fragments (scFv), single-domain antibody such as a heavy chain variable domain (VH), a light chain variable domain (VL) and a variable domain (VHH) of camelid derived single domain antibody, peptide, ligand or small molecule entity specific for HSA.

In other aspects, provided herein are pharmaceutical compositions comprising the bispecific antibody to CD33 and CD3, a vector comprising the polynucleotide encoding the polypeptide of the bispecific antibody to CD33 and CD3 or a host cell transformed by this vector and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents. In further aspects, the pharmaceutical compositions comprise excipients for sustained release, e.g. PLGA nanoparticles and the like. In further aspects, the pharmaceutical compositions are coated on a device for insertion into the body for sustained release at a particular site.

Bispecific antibodies to CD33 and CD3 with high-affinity binding to CD33 and CD3 are highly active in a large number of primary AML specimens, suggesting that these molecules could be active against human AML across the entire cytogenetic/molecular disease spectrum, even in cases of minimal CD33 expression. Of note, drug-specific cytotoxicity is also observed in the presence of residual autologous T-cells and is significantly augmented by the addition of controlled amounts of healthy donor T-cells (see Example 6).

The bispecific antibodies to CD33 and CD3, in particular 2×2 T-cell engagers, can induce potent cytolysis of CD33⁺ leukemic cells in vitro. The data indicate that high-affinity binding to both CD33 and CD3 maximizes bispecific protein-induced T-cell activation and anti-AML efficacy. High-affinity CD33/CD3-directed bispecific binding proteins, such as the 2×2 T-cell engagers described herein display cytolysis activity in primary AML in vitro. Thus, these bispecific antibodies to CD33 and CD3, in particular 2×2 T-cell engagers are suitable for a therapeutic approach for the treatment of diseases and conditions that are exacerbated or mediated by MDSCs such as inflammatory disease and conditions.

### Inflammatory Diseases and Conditions

Provided herein are methods wherein a bispecific antibody to CD33 and CD3 as described herein above is administered in an effective dose to a subject, e.g., a patient, for the treatment of an inflammatory disease or condition. Inflammatory disease or conditions include autoimmune diseases and heteroimmune conditions.

Autoimmune diseases are characterized with pathogenic autoantibody production as well as immune-complex mediated activation of Fc-gamma signaling pathways resulting in pro-inflammatory cytokine production of effector cells (macrophages, neutrophils, mast cells) leading to tissue destruction. Autoimmune disease include, but are not limited to, rheumatoid arthritis, psoriatic arthritis, osteoarthritis, Still's disease, juvenile idiopathic arthritis, lupus, diabetes, myasthenia gravis, Hashimoto's thyroiditis, Ord's thyroiditis, Graves' disease Sjogren's syndrome, multiple sclerosis, Guillain-Barre syndrome, acute disseminated encephalomyelitis, Addison's disease, opsoclonus-myoclonus syndrome, ankylosing spondylitis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia. In some embodiments, the methods described herein include administering to a subject a bispecific antibody to CD33 and CD3 for treating an autoimmune disease.

In some embodiments, the methods described herein are used to treat heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

In further embodiments, the methods described herein are used to treat an inflammatory disease, which includes, but is not limited to asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, gout, hepatitis, hidradenitis suppurativa, laryngitis, mastitis, meningitis, myelitis myocarditis, myositis, nephritis, Nonalcoholic steatohepatitis (NASH), oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, sepsis, salpingitis, sinusitis, stomatitis, synovitis, tendonitis, tonsillitis, uveitis, vaginitis, vasculitis, and vulvitis.

In some embodiments, the inflammatory disease or condition is caused by a pathogenic infection (e.g., viral, bacterial, parasitic or fungal). Many pathogenic infections elevate and promote the survival and accumulation of MDSCs and cause chronic inflammation. Pathogenic infective agents include, but are not limited to oncoviruses (e.g., human papillomavirus), human immunodeficiency virus (HIV), hepatitis B virus (HBV), vesicular stomatitis virus, respiratory syncytial virus (RSV), metapneumovirus (MPV), rhinovirus, influenza virus, parainfluenza virus, coronavirus, norovirus, rotavirus, hepatitis virus, adenovirus, astrovirus, Pseudomonas aeruginosa, S. aureus, methicillin-resistant S. aureus (MRSA), vancomycin-resistant enterococci (VRE), Enterococcus spp., Enterobacter spp., C. difficile, Campylobacter, E. faecali, E. faecium, or Salmonella, and the like.

In other embodiments, the bispecific antibody to CD33 and CD3 as described herein is administered for inhibiting or eliminating myeloid derived suppressor cells (MDSCs). In other embodiments, the bispecific antibody to CD33 and CD3 as described herein is administered for treating a condition associated with MDSCs. In yet other embodiments, the bispecific antibody to CD33 and CD3 as described herein is administered to treat immune suppression. In yet other embodiments, the bispecific antibody to CD33 and CD3 as described herein is administered to treat inflammation or immune suppression suppressed by MDSCs. In yet other embodiments, the bispecific antibody to CD33 and CD3 as described herein is administered to treat a decreased immune response caused by MDSCs.

### Dosing and Administration

The bispecific antibody to an antigen expressed on a target cell and an antigen expressed on a T-cell, e.g., CD33 and CD3, described herein is contemplated for use as a medicament. Administration is effected by different ways, e.g. by intravenous, intraperitoneal, subcutaneous, intramuscular, intralesional, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently.

An “effective dose” refers to amounts of the active ingredient that are sufficient to affect the course and the severity of the disease, leading to the reduction or remission of such pathology. For example, an “effective dose” useful for treating and/or preventing a CD33⁺ cancer such as AML may be determined using known methods. Maximum tolerated doses (MTD) and maximum response doses (MRD) can be determined via established animal and human experimental protocols as well as in the examples described herein.

In some embodiments, a bispecific antibody to an antigen expressed on a target cell and an antigen expressed on a T-cell, e.g., CD33 and CD3, described herein is provided in a dose per day, i.e., ‘continuous dose’, from about 0.01 μg to about 1000 μg, from about 0.05 μg to about 500 μg, from about 0.1 μg to about 500 μg, or about 0.5 μg to about 300 μg. In certain embodiments, a bispecific antibody to CD33 and CD3 described herein is provided in a daily dose or continuous dose of about 0.01 μg, about 0.02 μg, about 0.05 μg, about 0.07 μg, about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1.5 μg, about 2 μg, about 2.5 μg, about 3 μg, about 4 μg, about 5 μg, about 6 μg, about 7 μg, about 8 μg, about 9 μg, about 10 μg, about 12 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 275 μg, about 300 μg, about 350 μg, about 400 μg, about 450 μg, about 500 μg, about 600 μg, about 700 μg, about 800 μg, about 900 μg, about 1000 μg, about 2000 μg, about 3000 μg, or more, or any range derivable therein. In certain instances, a bispecific antibody described herein is provided in a daily dose of about 0.5 μg. In certain instances, a bispecific antibody described herein is provided in a daily dose of about 1.5 μg. In certain instances, a bispecific antibody described herein is provided in a daily dose of about 5 μg. In certain instances, a bispecific antibody described herein is provided in a daily dose of about 15 μg. In certain instances, a bispecific antibody described herein is provided in a daily dose of about 50 μg. In certain instances, a bispecific antibody described herein is provided in a daily dose of about 100 μg. In certain instances, a bispecific antibody described herein is provided in a daily dose of about 150 μg. In certain instances, a bispecific antibody described herein is provided in a daily dose of about 200 μg. In certain instances, a bispecific antibody described herein is provided in a daily dose of about 250 μg. In certain instances, a bispecific antibody described herein is provided in a daily dose of about 300 μg. In certain instances, a bispecific antibody described herein is provided in a daily dose of about 500 μg. In certain instances, a bispecific antibody described herein is provided in a daily dose of about 1000 μg. In certain instances, a bispecific antibody described herein is provided in a daily dose of about 2000 μg. In certain instances, a bispecific antibody described herein is provided in a daily dose of about 3000 μg.

In further embodiments, a bispecific antibody to an antigen expressed on a target cell and an antigen expressed on a T-cell, e.g., CD33 and CD3, described herein is provided in a daily dose from about 0.0001 μg/kg to about 10 μg/kg per body weight. In certain instances, a bispecific described herein is provided in a daily dose of about 0.001 μg/kg, about 0.005 μg/kg, about 0.01 μg/kg, about 0.03 μg/kg, about 0.05 μg/kg, about 0.07 μg/kg, about 0.1 μg/kg, about 0.2 μg/kg, about 0.3 μg/kg, about 0.4 μg/kg, about 0.5 μg/kg, about 0.6 μg/kg, about 0.7 μg/kg, about 0.8 μg/kg, about 0.9 μg/kg, about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, or about 10 μg/kg, about 20 μg/kg, or about 30 μg/kg, or more, or any range derivable therein.

In further embodiments, a bispecific antibody to an antigen expressed on a target cell and an antigen expressed on a T-cell, e.g., CD33 and CD3, described herein is provided in a dose per day from about 0.005 μg/m² to about 500 μg/m² patient body surface area, from about 0.025 μg/m² to about 250 μg/m², from about 0.05 μg/m² to about 250 μg/m², or about 0.25 μg/m² to about 150 μg/m². In certain embodiments, a bispecific antibody described herein is provided in a daily dose of about 0.005 μg/m², about 0.01 μg/m², about 0.05 μg/m², about 0.1 μg/m², about 0.2 μg/m², about 0.3 μg/m², about 0.4 μg/m², about 0.5 μg/m², about 0.6 μg/m², about 0.7 μg/m², about 0.8 μg/m², about 0.9 μg/m², about 1 μg/m², about 1.5 μg/m², about 2 μg/m², about 2.5 μg/m², about 3 μg/m², about 4 μg/m², about 5 μg/m², about 6 μg/m², about 7 μg/m², about 8 μg/m², about 9 μg/m², about 10 μg/m², about 12 μg/m², about 15 μg/m², about 20 μg/m², about 25 μg/m², about 30 μg/m², about 35 μg/m², about 40 μg/m², about 45 μg/m², about 50 μg/m², about 60 μg/m², about 70 μg/m², about 80 μg/m², about 90 μg/m², about 100 μg/m², about 125 μg/m², about 150 μg/m², about 175 μg/m², about 200 μg/m², about 250 μg/m², about 275 μg/m², about 300 μg/m², about 350 μg/m², about 400 μg/m², about 450 μg/m², about 500 μg/m², about 600 μg/m², about 700 μg/m², about 800 μg/m², about 1000 μg/m², about 1200 μg/m², or about 1500 μg/m², or more, or any range derivable therein.

The dose per day described herein can be administered by bolus infusion or continuous infusion. Bolus infusion as used herein refers to an infusion which is interrupted earlier than 6 hours, whereas the term “continuous infusion” refers to an infusion which is allowed to proceed permanently for at least 6 hours without interruption. “Continuous infusion” refers to a permanently administered infusion. Accordingly, the terms “permanent” and “continuous” are used as synonyms. In some embodiments, a bispecific antibody to an antigen expressed on a target cell and an antigen expressed on a T-cell, e.g., CD33 and CD3, described herein is administered as a continuous infusion over 6 h, 7 h, 8 h, 9 h, 10 h, 11 h, 12 h, 14 h, 16, 18, 20, 22, or 24 h per day. In some embodiments, a bispecific antibody is administered as a 12 h continuous infusion. In some embodiments, a bispecific antibody is administered as a 24 h continuous infusion. The dose per day described herein can be given once per day or multiple times per day in the form of sub-doses given b.i.d., t.i.d., q.i.d., or the like where the number of sub-doses equal the dose per day. It is further contemplated that the dose per day described herein and/or its sub-doses can be administered at the same location site on a patient or different sites.

The dose per day described herein, in additional embodiments, can be administered at a certain infusion rates. In certain embodiments, a bispecific antibody to CD33 and CD3 described herein is infused at a rate of about 0.01 μg/h, about 0.02 μg/h, about 0.05 μg/h, about 0.07 μg/h, about 0.1 μg/h, about 0.2 μg/h, about 0.3 μg/h, about 0.4 μg/h, about 0.5 μg/h, about 0.6 μg/h, about 0.7 μg/h, about 0.8 μg/h, about 0.9 μg/h, about 1 μg/h, about 1.5 μg/h, about 2 μg/h, about 2.5 μg/h, about 3 μg/h, about 4 μg/h, about 5 μg/h, about 6 μg/h, about 7 μg/h, about 8 μg/h, about 9 μg/h, about 10 μg/h, about 12 μg/h, about 15 μg/h, about 20 μg/h, about 25 μg/h, about 30 μg/h, about 35 μg/h, about 40 μg/h, about 45 μg/h, about 50 μg/h, about 55 μg/h, about 60 μg/h, about 65 μg/h, about 70 μg/h, about 75 μg/h, about 80 μg/h, about 85 μg/h, about 90 μg/h, about 95 μg/h, about 100 μg/h μg, about 125 μg/h, about 150 μg/h, about 175 μg/h, about 200 μg/h, or more, or any range derivable therein. In certain instances, a bispecific antibody described herein is infused at a rate of about 0.25 μg/h. In certain instances, a bispecific antibody described herein is infused at a rate of about 0.5 μg/h. In certain instances, a bispecific antibody described herein is infused at a rate of about 0.75 μg/h. In certain instances, a bispecific antibody described herein is infused at a rate of about 1 μg/h. In certain instances, a bispecific antibody described herein is provided in a daily dose of about 1.5 μg/h. In certain instances, a bispecific antibody described herein is infused at a rate of about 2 μg/h. In certain instances, a bispecific antibody described herein is infused at a rate of about 2.5 μg/h. In certain instances, a bispecific antibody described herein is infused at a rate of about 3 μg/h. In certain instances, a bispecific antibody described herein is infused at a rate of about 4 μg/h. In certain instances, a bispecific antibody described herein is provided in a daily dose of about 5 μg. In certain instances, a bispecific antibody described herein is infused at a rate of about 6 μg/h. In certain instances, a bispecific antibody described herein is infused at a rate of about 7 μg/h. In certain instances, a bispecific antibody described herein is infused at a rate of about 7.5 μg/h. In certain instances, a bispecific antibody described herein is infused at a rate of about 8 μg/h. In certain instances, a bispecific antibody described herein is infused at a rate of about 9 μg/h. In certain instances, a bispecific antibody described herein is infused at a rate of about 10 μg/h. In certain instances, a bispecific antibody described herein is provided in a daily dose of about 15 μg/h. In certain instances, a bispecific antibody described herein is infused at a rate of about 20 μg/h. In certain instances, a bispecific antibody described herein is infused at a rate of about 25 μg/h. In certain instances, a bispecific antibody described herein is infused at a rate of about 30 μg/h. In certain instances, a bispecific antibody described herein is infused at a rate of about 40 μg/h. In certain instances, a bispecific antibody described herein is provided in a daily dose of about 50 μg/h. In certain instances, a bispecific antibody described herein is provided in a daily dose of about 60 μg/h. In certain instances, a bispecific antibody described herein is provided in a daily dose of about 70 μg/h. In certain instances, a bispecific antibody described herein is provided in a daily dose of about 80 μg/h. In certain instances, a bispecific antibody described herein is provided in a daily dose of about 90 μg/h. In certain instances, a bispecific antibody described herein is provided in a daily dose of about 100 μg/h.

It is further contemplated that the infusion rates can be variable to reduce the risk of side effects, such as cytokine release syndrome, or allows the subject to acclimate to the bispecific antibody. In some instances, an infusion rate can begin at a rate for a certain period of time, i.e., a lead-in dose, and then ‘stepped-up’ to a high rate. In some instances, an infusion rate can include two or more ‘stepped-up’ higher rates. In some instances, an infusion rate can begin at a certain rate, and then ‘stepped-down’ to a lower rate. In some instances, an infusion rate can include two or more ‘stepped-down’ lower rates. In further instances, an infusion rate can include both a ‘stepped-up’ and ‘stepped-down’ rates.

In further embodiments, administration of a bispecific antibody to an antigen expressed on a target cell and an antigen expressed on a T-cell, e.g., CD33 and CD3 is at doses described herein or at other dose levels determined and contemplated by a medical practitioner. In certain therapeutic applications, a bispecific antibody is administered to a patient already suffering from a cancer, in an amount sufficient to cure or at least partially arrest the symptoms of the cancer. Amounts effective for this use depend on the severity and course of the cancer, previous therapy, the patient's health status, weight, and response to the drugs, and the judgment of the treating physician. Therapeutically effective amounts are optionally determined by methods including, but not limited to, a dose escalation clinical trial, such as described in the following example.

In some embodiments, a bispecific antibody to an antigen expressed on a target cell and an antigen expressed on a T-cell, e.g., CD33 and CD3, described herein is administered continuously or chronically, i.e., daily dosing for a particular amount of time or cycle. In some embodiments, a bispecific antibody described herein is administered at least 1 week (7 days), at least 2 weeks (14 days), at least 3 weeks (21 days), at least 4 weeks, at least 6 weeks, at least 8 weeks, at least 12 weeks, least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 90 weeks, at least about 100 weeks, at least about 110 weeks, and at least about 120 weeks.

Administration periods can be further defined as treatment cycles where a given number of days or weeks equates one treatment cycle. In some embodiments, one treatment cycle is an administration period of about 1 week, about 2 weeks, about 4 weeks, about 6 weeks, about 8 weeks, about 10 weeks, about 12 weeks or about 16 weeks. In certain embodiments, one treatment cycle is 2 weeks. Treatment cycles for administration of a bispecific antibody to an antigen expressed on a target cell and an antigen expressed on a T-cell, e.g., CD33 and CD3, described herein also include, but are not limited to 1 cycle, 2 cycles, 3 cycles, 4 cycles, 5 cycles, 6 cycles, 7 cycles, 8 cycles, 9 cycles, 10 cycles, 11 cycles, 12 cycles, 13 cycles, 14 cycles, 15 cycles, 16 cycles, 17 cycles, 18 cycles, 19 cycles, 20 cycles, 25 cycles, 30 cycles, 40 cycles, or more.

Dosages for a bispecific antibody to an antigen expressed on a target cell and an antigen expressed on a T-cell, e.g., CD33 and CD3, described herein can, in some embodiments, be the same for each treatment cycle or the dosages may vary per cycle. In some embodiments, a higher initial dose of a bispecific antibody described herein is administered for the first cycle and a lower dose is administered for all subsequent cycles. In other embodiments, the dosages are decreased gradually per administration for each cycle. In yet other embodiments, the dosages are increased gradually per administration for each cycle.

In some embodiments, administration for a bispecific antibody to an antigen expressed on a target cell and an antigen expressed on a T-cell, e.g., CD33 and CD3, described herein is withheld or given a “drug holiday” in one or more treatment cycles. For example, a bispecific antibody described herein is administered for one treatment cycle and subsequently withheld for the next treatment cycle. In other embodiments, a bispecific antibody described herein is withheld from a subject every other treatment cycle, every two treatment cycles, every three treatment cycles, every four treatment cycles, or every five treatment cycles.

Administration of a bispecific antibody to an antigen expressed on a target cell and an antigen expressed on a T-cell, e.g., CD33 and CD3, described herein can, in other embodiments, also be provided in an intermittent dosing schedule. Intermittent dosing schedules include administering a bispecific antibody described herein for a number of days, withholding administration for a certain period of time, subsequently administering the bispecific antibody again with another subsequent withholding. Intermittent dosing can be used to stay within the safety profile as well as maximize efficacy potential of the bispecific antibody. In a non-limiting example, for a 14-day treatment cycle, a bispecific antibody can be administered for days 1-4 and 8-12. Another intermittent dosing schedule is administration of a bispecific antibody every other day. Other intermittent dosing schedules are contemplated that include administration of a bispecific antibody daily for one, two, three, four, five, six, seven, eight, nine or ten days, a withholding period of one, two, three, four, five, six, seven, eight, nine or ten days and an optional daily and withholding period similar or different from the previous administration within a treatment cycle. In a non-limiting example, a bispecific antibody described herein is administered daily for three days at a certain dose and then subsequently every other day at the same or different dose of a particular treatment period or cycle (See e.g., FIG. 24, bottom).

In further embodiments, administration of a bispecific antibody to an antigen expressed on a target cell and an antigen expressed on a T-cell, e.g., CD33 and CD3, described herein is provided once a week, twice a week, three times a week, four times a week, five times a week or six times a week. In certain instances, administration of a bispecific antibody is provided once a week. In certain instances, administration of a bispecific antibody is provided twice a week. In certain instances, administration of a bispecific antibody is provided three times a week.

In certain embodiments, intermittent dosing is combined with dose titration. Dose titration refers to administration of a bispecific antibody at certain dosage and then increasing the dosage after the intermittent period. The dose can be titrated one, two, three, four, or five times. For example, an every other day intermittent dosing can have a dose titration of 5→15→100 μg where the 100 μg dose is reached on the fifth and subsequent days (FIG. 24, top).

### Pharmacokinetics and Pharmacodynamics

The dosing and administration regimens for a bispecific antibody to an antigen expressed on a target cell and an antigen expressed on a T-cell, e.g., CD33 and CD3, provided herein further provide unique pharmacokinetic profiles not seen with other therapeutic proteins or bispecific antibodies that bind to and engage T cells.

Many therapeutic proteins have rapid clearance and short half-lives. Examples of such proteins which are commercially marketed include interferon alfa-2a (Roferon-A®, half-life: 3.7-8.5 h, MW 19 kDa), filgrastim (Neupogen®, half-life:3.5 h, MW 18 kDa), and imiglucerase (Cerezyme®, half-life: 4-10 min, MW 60 kDa). Many bispecific antibodies also have rapid clearance and short half-life. For example, blinatumomab, an anti-CD19×CD3 bispecific BiTE® antibody (MW 54 kDa) has a half-life of around 1-2 h.

The rapid clearance and short half-life of certain proteins and antibodies can require more frequent or longer dosing regimens. Current methods that increase half-life include the addition of Fc domains to encourage FcRn recycling of the protein, modifications such as glycosylation or pegylation, or linkage or binding to serum proteins such as albumin. In contrast, the bispecific antibodies to an antigen expressed on a target cell and an antigen expressed on a T-cell described herein, e.g., CD33 and CD3, have long half-lives of approximately one to two days when administered to a human subject. In some embodiments, the bispecific antibodies to an antigen expressed on a target cell and an antigen expressed on a T-cell described herein have a half-life of greater than 2 h, about 3 h, about 4 h, about 6 h, about 8, about h, about 10 h, about 12 h, about 14 h, about 16 h, about 18 h, about 20 h, about 22 h, about 24 h, about 30 h, about 36 h, about 40 h, about 44 h, about 48 h, or greater than 48 h. This is remarkable in that the bispecific antibodies to an antigen expressed on a target cell and an antigen expressed on a T-cell described herein, e.g., CD33 and CD3, are not designed with half-life extension methods such as the ones previously described. The long half-lives of the bispecific antibodies described herein present therapeutic benefits such as less frequent dosing, lower dosing and having a prolonged effective concentration during or after a treatment cycle. In some embodiments, the bispecific antibodies described herein have a half-life of about 48 h or 2 days.

In another aspect, the administration of the bispecific antibodies to CD33 and CD3 further provide a consistent increase in drug concentration with a maximum concentration (Cₘₐₓ) in the blood achieved in about 1 to 21 days. In some embodiments, the administration of the bispecific antibodies to CD33 and CD3 further provide a Cₘₐₓ in 1 day, in 2 days, in in 3 days, in 4 days, in 5 days, in 6 days, in 7 days, in 8 days, in 9 days, in 10 days, in 11 days, in 12 days, in 13 days, in 14 days, in 15 days, in 16 days, in 17 days, in 18 days, in 19 days, in 20 days, or in 21 days. In some embodiments, the administration of the bispecific antibodies to CD33 and CD3 further provide a Cₘₐₓ in 1 day.

In another aspect, the administration of the bispecific antibodies to CD33 and CD3 further provide a consistent increase in drug concentration with a steady state concentration (Cₛₛ) in the blood achieved in about 1 to 21 days. In some embodiments, the administration of the bispecific antibodies to CD33 and CD3 further provide a Cₛₛ in 1 day, in 2 days, in in 3 days, in 4 days, in 5 days, in 6 days, in 7 days, in 8 days, in 9 days, in 10 days, in 11 days, in 12 days, in 13 days, in 14 days, in 15 days, in 16 days, in 17 days, in 18 days, in 19 days, in 20 days, or in 21 days. In some embodiments, the administration of the bispecific antibodies to CD33 and CD3 further provide a Cₛₛ in 1 day. In some embodiments, the administration of the bispecific antibodies to CD33 and CD3 further provide a Cₛₛ in 1 to 3 days. In some embodiments, the administration of the bispecific antibodies to CD33 and CD3 further provide a Cₛₛ in 3 to 7 days. In some embodiments, the administration of the bispecific antibodies to CD33 and CD3 further provide a Cₛₛ in 1 to 7 days. In some embodiments, the administration of the bispecific antibodies to CD33 and CD3 further provide a Cₛₛ in 7 to 14 days. In some embodiments, the administration of the bispecific antibodies to CD33 and CD3 further provide a Cₛₛ in 3 to 14 days. In some embodiments, the administration of the bispecific antibodies to CD33 and CD3 further provide a Cₛₛ in 14 to 21 days. In some embodiments, the administration of the bispecific antibodies to CD33 and CD3 further provide a Cₛₛ in 7 to 21 days.

In yet another aspect, the administration of the bispecific antibodies to CD33 and CD3 described herein further provide a consistent increase in drug concentration with a Cₘₐₓ and a steady state concentration (Cₛₛ) in the blood achieved in about 1 to 21 days. In some embodiments, the administration of the bispecific antibodies to CD33 and CD3 described herein further provide a Cₘₐₓ and a Cₛₛ 1 day, in 2 days, in in 3 days, in 4 days, in 5 days, in 6 days, in 7 days, in 8 days, in 9 days, in 10 days, in 11 days, in 12 days, in 13 days, in 14 days, in 15 days, in 16 days, in 17 days, in 18 days, in 19 days, in 20 days, or in 21 days. In some embodiments, the administration of the bispecific antibodies to CD33 and CD3 described herein further provide a Cₘₐₓ and a Cₛₛ in 1 day.

In some embodiments, the bispecific antibodies to CD33 and CD3 described herein are administered at a dose and frequency to provide a Cₘₐₓ of about 10 pg/mL, about 20 pg/mL, about 30 pg/mL, about 40 pg/mL, about 50 pg/mL, about 60 pg/mL, about 70 pg/mL, about 80 pg/mL, about 90 pg/mL, about 100 pg/mL, about 150 pg/mL, about 200 pg/mL, about 250 pg/mL, about 300 pg/mL, about 350 pg/mL, about 400 pg/mL, about 500 pg/mL, about 600 pg/mL, about 00 pg/mL, about 800 pg/mL, about 900 pg/mL, about 1000 pg/mL, about 2000 pg/mL, about 3000 pg/mL, about 4000 pg/mL, about 5000 pg/mL, about 6000 pg/mL, about 7000 pg/mL, about 8000 pg/mL, about 9000 pg/mL, or about 10000 pg/mL.

In some embodiments, the bispecific antibodies to CD33 and CD3 described herein are administered at a dose and frequency to provide a Cₛₛ of about 10 pg/mL, about 20 pg/mL, about 30 pg/mL, about 40 pg/mL, about 50 pg/mL, about 60 pg/mL, about 70 pg/mL, about 80 pg/mL, about 90 pg/mL, about 100 pg/mL, about 150 pg/mL, about 200 pg/mL, about 250 pg/mL, about 300 pg/mL, about 350 pg/mL, about 400 pg/mL, about 500 pg/mL, about 600 pg/mL, about 00 pg/mL, about 800 pg/mL, about 900 pg/mL, about 1000 pg/mL, about 2000 pg/mL, about 3000 pg/mL, about 4000 pg/mL, about 5000 pg/mL, about 6000 pg/mL, about 7000 pg/mL, about 8000 pg/mL, about 9000 pg/mL, or about 10000 pg/mL.

In further embodiments, the bispecific antibodies to CD33 and CD3 described herein are administered at a dose and frequency to provide an AUC of about 100 day*pg/mL, about 200 day*pg/mL, about 300 day*pg/mL, about 400 day*pg/mL, about 500 day*pg/mL, about 600 day*pg/mL, about 700 day*pg/mL, about 800 day*pg/mL, about 900 day*pg/mL, about 1000 day*pg/mL, about 2000 day*pg/mL, about 3000 day*pg/mL, about 4000 day*pg/mL, about 5000 day*pg/mL, about 6000 day*pg/mL, about 7000 day*pg/mL, about 8000 day*pg/mL, about 9000 day*pg/mL, 10000 day*pg/mL, 20000 day*pg/mL, 30000 day*pg/mL, 40000 day*pg/mL, 50000 day*pg/mL, 60000 day*pg/mL, 70000 day*pg/mL, 80000 day*pg/mL, 90000 day*pg/mL, or 100000 day*pg/mL.

In another aspect, the administration of the bispecific antibodies to CD33 and CD3 described herein produce desirable pharmacodynamics profiles as compared to existing bispecific antibodies. As discussed previously, a common phenomenon observed in antibody therapy is the occurrence of CRS. For example, the initial administration of blinatumomab provides a rapid increase in cytokine release with elevated levels of IL-10, IL-6, IFN-γ, TNFα, and IL-2 present on day 1. The initial cytokine release is also dose-dependent. This reported observation has led to a stepped dosing regimen for blinatumomab, where initial low dosing is required to reduce initial cytokine release.

It is further contemplated the administration of the bispecific antibodies to CD33 and CD3 described herein provides for a controlled or gradual cytokine release in contrast to blinatumomab and other bispecific antibodies. Such cytokines include, but are not limited to, TNFα, IL-2, IL-4, IL-6, IL-8, IL-10, TGFβ, and IFNγ. Furthermore, it is contemplated that the administration of the bispecific antibodies to CD33 and CD3 described herein provides for controlled T cell expansion and/or activation. In some embodiments, the administration of the bispecific antibodies to CD33 and CD3 described herein prevents short-term, burst-like T cell activation. In some embodiments, the administration of the bispecific antibodies to CD33 and CD3 described herein promotes long term T cell activation and expansion.

In another aspect, the administration of the bispecific antibodies to CD33 and CD3 described herein reduce inflammation. Inflammation can be measured via markers such as C-reactive protein (CRP) levels in the blood or serum, or other tests such as erythrocyte sedimentation rate (ESR) or plasma viscosity (PV). A raised or elevated CRP (or ESR or PV) level is an indication of inflammation. Many subjects with cancer also have elevated CRP levels. In some embodiments, the administration of the bispecific antibodies to CD33 and CD3 described herein reduces CRP levels by about 10%, about 20%, about 30%, about 40%, about 50%, about 60%, about 70%, about 80%, about 90%, or by about 100%. In some embodiments, the administration of the bispecific antibodies to CD33 and CD3 described herein reduces CRP levels to about 90 mg/L, about 80 mg/L, about 70 mg/L, about 60 mg/L, about 50 mg/L, about 40 mg/L, about 30 mg/L, about 20 mg/L, about 10 mg/L, about 5 mg/L, about 2 mg/L, or about 1 mg/L. In some embodiments, the administration of the bispecific antibodies to CD33 and CD3 described herein reduces CRP levels to normal levels (e.g., about 5 to about 10 mg/L).

In another aspect, the administration of the bispecific antibodies to CD33 and CD3 described herein promote, restore, or regenerate hematopoiesis. In another aspect, the administration of the bispecific antibodies to CD33 and CD3 described herein promote, restore, or regenerate myelopoiesis. In some embodiments, the administration of the bispecific antibodies to CD33 and CD3 described herein increase hematopoietic stem cells. In some embodiments, the administration of the bispecific antibodies to CD33 and CD3 described herein increase myeloid cells, which include monocytes, macrophages, neutrophils, basophils, eosinophils, erythrocytes, dendritic cells, megakaryocytes, or platelets. In some embodiments, the administration of the bispecific antibodies to CD33 and CD3 described herein increase lymphoid cells (e.g., T cells, B cells, and NK cells).

In certain embodiments, the administration of the bispecific antibodies to CD33 and CD3 described herein increase absolute neutrophil counts by about 10%, about 120%, about 30%, about 40%, about 50%, about 60%, about 70%, about 80%, about 90%, or about 100%, or more. In certain embodiments, the administration of the bispecific antibodies to CD33 and CD3 described herein increase absolute neutrophil counts to about 0.1×10⁹/L, about 0.2×10⁹/L, about 0.3×10⁹/L, about 0.4×10⁹/L, about 0.5×10⁹/L, about 0.6×10⁹/L, about 0.7×10⁹/L, about 0.8×10⁹/L, about 0.9×10⁹/L, about 1×10⁹/L, about 1.5×10⁹/L, about 2 x109/L, about 2.5×10⁹/L, about 3×10⁹/L, about 3.5×10⁹/L, about 4×10⁹/L, about 4.5×10⁹/L, about 5×10⁹/L, about 5.5×10⁹/L, about 6×10⁹/L, about 6.5×10⁹/L, about 7×10⁹/L, about 7.5×10⁹/L, or about 8×10⁹/L, or more. In certain embodiments, the administration of the bispecific antibodies to CD33 and CD3 described herein increase absolute neutrophil counts to normal levels (e.g., about 2×10⁹/L to about 8×10⁹/L).

In certain embodiments, the administration of the bispecific antibodies to CD33 and CD3 increase monocyte counts by about 10%, about 120%, about 30%, about 40%, about 50%, about 60%, about 70%, about 80%, about 90%, or about 100%, or more. In certain embodiments, the administration of the bispecific antibodies to CD33 and CD3 increase monocyte counts to about 0.05×10⁹/L, about 0.1×10⁹/L, about 0.15×10⁹/L, about 0.2×10⁹/L, about 0.2.5×10⁹/L, about 0.3×10⁹/L, about 0.4×10⁹/L, about 0.5×10⁹/L, about 0.6×10⁹/L, about 0.7×10⁹/L, about 0.8×10⁹/L, about 0.9-10⁹/L, about 1×10⁹/L, or more. In certain embodiments, the administration of the bispecific antibodies to CD33 and CD3 increase absolute neutrophil counts to normal levels (e.g., about 0.2×10⁹/L to about 1×10⁹/L).

In certain embodiments, the administration of the bispecific antibodies to CD33 and CD3 increase platelet levels. In certain embodiments, the administration of the bispecific antibodies to CD33 and CD3 increase platelet counts to about 40×10⁹/L, about 50×10⁹/L, about 60×10⁹/L, about 70×10⁹/L, about 80×10⁹/L, about 90×10⁹/L, about 100×10⁹/L, about 125×10⁹/L, about 150×10⁹/L, about 175×10⁹/L, about 200×10⁹/L, about 225×10⁹/L, about 250×10⁹/L, about 275×10⁹/L, about 300×10⁹/L, about 325×10⁹/L, about 350×10⁹/L, about 375×10⁹/L, about 400 10⁹/L, about 450×10⁹/L, about 475×10⁹/L, about 500×10⁹/L, or more. In certain embodiments, the administration of the bispecific antibodies to CD33 and CD3 increase platelet counts to normal levels (e.g., about 150×10⁹/L to about 450×10⁹/L).

In certain embodiments, the administration of the bispecific antibodies to CD33 and CD3 described herein increase erythrocyte levels. Erythrocyte levels can be determined by hemoglobin concentration. In certain embodiments, the administration of the bispecific antibodies to CD33 and CD3 described herein increase hemoglobin concentration by about 10%, about 120%, about 30%, about 40%, about 50%, about 60%, about 70%, about 80%, about 90%, or about 100%, or more. In certain embodiments, the administration of the bispecific antibodies to CD33 and CD3 described herein increase hemoglobin concentration to about 8 g/dL, about 8.5 g/dL, about 9 g/dL, about 9.5 g/dL, about 10 g/dL, about 10.5 g/dL, about 11 g/dL, about 11.5 g/dL, about 12 g/dL, about 12.5 g/dL, about 13 g/dL, about 13.5 g/dL, about 14 g/dL, about 14.5 g/dL, about 15 g/dL, about 15.5 g/dL, about 16 g/dL, about 16.5 g/dL, about 17 g/dL, about 17.5 g/dL, about 18 g/dL, about 18.5 g/dL, about 19 g/dL, about 19.5 g/dL, or about 20 g/dL, or more. In certain embodiments, the administration of the bispecific antibodies to CD33 and CD3 described herein increase hemoglobin concentration to normal levels (about 12 to about 18 g/dL).

In another aspect, the administration of the bispecific antibodies to CD33 and CD3 described herein reduce the level of myeloblasts in subjects having AML. In certain embodiments, the bispecific antibodies to CD33 and CD3 described herein reduce the level of myeloblasts by about 10%, about 20%, about 30%, about 40%, about 50%, about 60%, about 70%, about 80%, about 90%, or by about 100%. In certain embodiments, the bispecific antibodies to CD33 and CD3 described herein control the level of myeloblasts, wherein the myeloblasts do not increase in their levels.

In another aspect, the administration of the bispecific antibodies to CD33 and CD3 described herein reduce the level of myeloid-derived suppressor cells (MDSCs). In certain embodiments, the bispecific antibodies to CD33 and CD3 described herein reduce the level of MDSCs by about 10%, about 20%, about 30%, about 40%, about 50%, about 60%, about 70%, about 80%, about 90%, or by about 100%.

### Further Combinations

In further embodiments, the bispecific antibody to CD33 and CD3 described herein is administered in combination with a standard therapy to inflammatory disease or conditions. Standard therapies include steroids, NSAIDs, COX-2 inhibitors, anti-TNFα agents, cytokine suppressive anti-inflammatory drug(s) (CSAIDs), other anti-inflammatory agents and the like.

The examples below further illustrate the described embodiments without limiting the scope of the invention.

### Example 1

### Cloning of DNA Expression Constructs Encoding Single-Chain Fv Antibodies

For bacterial expression of anti-CD33 single-chain Fv (scFv) antibodies in E. coli, DNA coding sequences of all molecules were cloned into a bacterial expression vector. All expression constructs were designed to contain coding sequences for an N-terminal signal peptide and C-terminal hexa-histidine (6×His)-tag to facilitate antibody secretion into the periplasm and purification, respectively. The amino acid sequences of the VL and VH-domains from all anti-CD33 scFv clones are shown in Table 3 and Table 4.

Expression of Recombinant Anti-CD33 Single-Chain Fv Antibodies in E. coli

Recombinant scFv antibodies were expressed as soluble secreted proteins in the E. coli periplasm. In a first step a small medium culture supplemented with ampicillin was inoculated with transformed bacteria and incubated for 16 h at 28° C. Subsequently, optical density was adjusted by adding a second medium supplemented with ampicillin and incubated once more at 28° C. until an optical density in the range of 0.6-0.8 at 600 nm was reached. Protein expression was induced through addition of 50 pM IPTG and incubation of cultures at 21-28° C. and 200 rpm for up to 16 h. Following incubation, cells were pelleted (30 min, 4° C., 7500 rpm) and stored at −20° C. until further processing.

### Purification of Anti-CD33 Single-Chain Fv Antibodies

Recombinant scFv were extracted from E. coli periplasm following centrifugation of bacterial cell cultures by resuspending cell pellets in buffer and incubation for 30 min at room temperature with gentle agitation. Cells were pelleted and supernatants containing recombinant proteins were kept. The procedure was repeated once more before supernatants were pooled and homogenized by ultrasonication. Homogenates were diluted, supplemented with low concentrations of imidazole and loaded onto a prepacked immobilized metal affinity chromatography (IMAC) column (GE Healthcare). The column was washed until baseline was reached and bound protein was then eluted with an imidazole buffer. Antibody containing fractions were pooled and subsequently purified by size-exclusion chromatography (SEC). Finally, protein eluates were concentrated by ultrafiltration and dialysed against storage buffer. Subsequent to low pH treatment (incubation at pH 3.0 for 20-24 h at 37° C.), samples were neutralized using Tris. Purified proteins were stored as aliquots at −80° C. until use.

### Example 2

### Cloning of DNA Expression Constructs Encoding 2×2 T-Cell Engagers

For expression of bispecific 2×2 T-cell engagers in CHO cells, coding sequences of all molecules were cloned into a mammalian expression vector system. The anti-CD33 scFv domains of Example 1 were used to construct CD33/CD3 2×2 T-cell engagers in combination with an anti-CD3 scFv domain, with domains organized as shown in Table 7 and FIG. 3. In brief, gene sequences encoding anti-CD33 VH and VL domains separated by a peptide linker (VH-linker-VL or VL-linker-VH) were synthesized and subcloned. The resulting construct was digested to generate separate VH and VL coding sequences utilizing a Bam HI restriction site located within the linker sequence. These VH and VL fragments were then ligated with a DNA fragment encoding VH and VL domains of anti-CD3 (VH-linker-VL or VL-linker-VH) to yield the final construct. Domain order variants 1 to 3 of CD33/CD3 2×2 T-cell engagers are shown in FIG. 3. All expression constructs were designed to contain coding sequences for an N-terminal signal peptide and a C-terminal hexahistidine (6×His)-tag to facilitate antibody secretion and purification, respectively.

### Expression of 2×2 T-Cell Engagers in Stably Transfected CHO Cells

A CHO cell expression system (Flp-In®, Life Technologies), a derivative of CHO-K1 Chinese Hamster ovary cells (ATCC, CCL-61) (Kao and Puck, Proc. Natl. Acad Sci USA 1968; 60(4):1275-81), was used. Adherent cells were subcultured according to standard cell culture protocols provided by Life Technologies.

For adaption to growth in suspension, cells were detached from tissue culture flasks and placed in serum-free medium. Suspension-adapted cells were cryopreserved in medium with 10% DMSO.

Recombinant CHO cell lines stably expressing secreted 2-2 T-cell engagers were generated by transfection of suspension-adapted cells. During selection with the antibiotic Hygromycin B viable cell densities were measured twice a week, and cells were centrifuged and resuspended in fresh selection medium at a maximal density of 0.1×10⁶ viable cells/mL. Cell pools stably expressing 2×2 T-cell engagers were recovered after 2-3 weeks of selection at which point cells were transferred to standard culture medium in shake flasks. Expression of recombinant secreted proteins was confirmed by performing protein gel electrophoresis or flow cytometry. Stable cell pools were cryopreserved in DMSO containing medium.

2×2 T-cell engagers were produced in 10-day fed-batch cultures of stably transfected CHO cell lines by secretion into the cell culture supernatant. Cell culture supernatants were harvested after 10 days at culture viabilities of typically >75%. Samples were collected from the production cultures every other day and cell density and viability were assessed. On day of harvest, cell culture supernatants were cleared by centrifugation and vacuum filtration before further use.

Protein expression titers and product integrity in cell culture supernatants were analyzed by SDS-PAGE.

### Purification of 2×2 T-Cell Engagers

2×2 T-cell engagers were purified from CHO cell culture supernatants in a two-step procedure. The His6-tagged constructs were subjected to Ni-NTA Superflow chromatography in a first step followed by preparative size exclusion chromatography (SEC) on Superdex 200 in a second step. Eluted 2×2 T-cell engagers were characterized with regards to their homodimer (2×2 T-cell engagers) content and pooled if the homodimer content was 90% or higher. Finally, pooled samples were buffer-exchanged and concentrated by ultrafiltration to a typical concentration of >1 mg/mL. Purity and homogeneity (typically >90%) of final samples were assessed by SDS PAGE under reducing and non-reducing conditions, followed by immunoblotting using an anti-His-Tag antibody as well as by analytical SEC, respectively. Purified proteins were stored at aliquots at −80° C. until use.

Polypeptides of CD33/CD3 2×2 T-cell engagers are shown in Table 7 and FIG. 3. Each 2×2 T-cell engager consists of two identical polypeptides (FIG. 1). Both outer linkers L1 and L3 were comprised of six amino acids GGSGGS (SEQ ID NO:95), whereas the central peptide linker 2 varied in length (4-6 amino acids) with the sequences GGSG (SEQ ID NO:96), GGSGG (SEQ ID NO:97), or GGSGGS (SEQ ID NO:95), respectively.

Using a series of anti-CD33 variable domains and anti-CD3 variable domains a large number of 2×2 T-cell engager molecules was generated that could be stably produced in transfected cell lines and that maintained stability at body temperature as well as after repeated freeze/thaw cycles. To facilitate further development and preclinical toxicology studies, emphasis was placed on the selection of 2×2 T-cell engager molecules that showed binding to both human and cynomolgus monkey CD33. Examples of complete amino acid sequences are shown for the single-chain of 2×2 T-cell engagers 12 (SEQ ID NO:109), 14 (SEQ ID NO:111) and 16 (SEQ ID NO:113) in FIGS. 10L, 10N and 10P, respectively. In this example the order of the variable domains and their linkers for the structures is: VL (CD3)-L1-VH (CD33)-L2-VL (CD33)-L3-VH (CD3). The C-terminal hexa-histidine (6×His)-tag is cleaved during purification. Complete amino acid sequences for the above mentioned 2-2 T-cell engagers, after removal of the hexa-histidine tag, are tandom diabody 12 (SEQ ID NO:134), 2×2 T-cell engager 14 (SEQ ID NO:136) and 2×2 T-cell engager 16 (SEQ ID NO:138), as shown in FIGS. 11L, 11N and 11P, respectively.

### Example 3

### Determination of Antibody Affinity by Flow Cytometry

Cells were incubated with 100 μL of serial dilutions of CD33/CD3 2×2 T-cell engagers. After washing three times with FACS buffer the cells were incubated with 0.1 mL of 10 pg/mL mouse monoclonal anti-His antibody in the same buffer for 45 min on ice. After a second washing cycle, the cells were incubated with 0.1 mL of 15 pg/mL FITC-conjugated goat anti-mouse IgG antibodies under the same conditions as before. As a control, cells were incubated with the anti-His IgG followed by the FITC-conjugated goat anti-mouse IgG antibodies without anti-CD33 2×2 T-cell engagers. The cells were then washed again and resuspended in 0.2 mL of FACS buffer containing 2 pg/mL propidium iodide (PI) in order to exclude dead cells. The fluorescence of 1×10⁴ living cells was measured using a Beckman-Coulter FC500 MPL flow cytometer using the MXP software (Beckman-Coulter, Krefeld, Germany) or a Millipore Guava EasyCyte flow cytometer using the Incyte software (Merck Millipore, Schwalbach, Germany). Mean fluorescence intensities of the cell samples were calculated using CXP software (Beckman-Coulter, Krefeld, Germany) or Incyte software (Merck Millipore, Schwalbach, Germany). After subtracting the fluorescence intensity values of the cells stained with the secondary and tertiary reagents alone the values were used for calculation of the K_{D} values with the equation for one-site binding (hyperbola) of the GraphPad Prism (version 6.00 for Windows, GraphPad Software, La Jolla Calif. USA).

The 2×2 T-cell engagers were tested for their binding affinities to human CD3⁺ and CD33⁺ cells and cynomolgus CD3⁺ and CD33⁺ cells. Exemplary binding data for selected 2×2 T-cell engagers are summarized in Table 8:

TABLE 8
CD3 and CD33 binding characteristics of CD33/CD3 2 × 2
T-cell engagers:

| 2 × 2 | K_{D} on | K_{D} on | K_{D} on | K_{D} on | K_{D} ratio | EC₅₀ on |
|---|---|---|---|---|---|---|
| T-cell | T cells | HL-60 | KG-1 | U-937 | cynoCD33/ | HL-60 |
| engagers | [nM] | [nM] | [nM] | [nM] | huCD33 | [pM] |
| 01 | 94.2 | 0.6 | 0.9 | 7.1 | 0.7 | 1.9 |
| 02 | 69.8 | 0.2 | 0.3 | 0.9 | 1.1 | 0.5 |
| 03 | 81.9 | 1.1 | 1.8 | 8.9 | 0.6 | 3.6 |
| 04 | 79.3 | 0.5 | 0.5 | 1.7 | 1.1 | 1.8 |
| 05 | 69.5 | 1.0 | 1.2 | 6.2 | 0.8 | 2.7 |
| 06 | 86.3 | 0.4 | 0.5 | 1.6 | 0.8 | 1.6 |
| 07 | 49.7 | 13.7 | 47.9 | 47.1 | 45.8 | 17.8 |
| 08 | 2.4 | 0.3 | 0.5 | 1.8 | 0.6 | 1.8 |
| 09 | 2.4 | 0.5 | 0.3 | 2.2 | 1.0 | 6.8 |
| 10 | 1.9 | 0.5 | 1.0 | 1.7 | 0.8 | 7.0 |
| 11 | 2.6 | 0.3 | 0.5 | 0.6 | 1.2 | 5.9 |
| 12 | 1.5 | 0.3 | 0.9 | 0.5 | 1.7 | 1.3 |
| 13 | 55.7 | 0.2 | 0.3 | 0.5 | 1.6 | 1.1 |
| 14 | 2.1 | 0.3 | 0.3 | 1.2 | 1.0 | 11.6 |
| 15 | 1.3 | 0.4 | 0.3 | 0.9 | 1.1 | 1.8 |
| 16 | 2.1 | 0.3 | 0.2 | 0.3 | 1.4 | 1.5 |
| 17 | 3.3 | 5.0 | 52.5 | 24.4 | 1.9 | 18.4 |
| 18 | 11.9 | 3.4 | 16.3 | 15.1 | 3.1 | 6.3 |
| 19 | 6.3 | 2.8 | 3.6 | 5.4 | 37.3 | 5.7 |
| 20 | 143.8 | 4.1 | 7.0 | 7.2 | 33.8 | 10.0 |
| 21 | 2.1 | 9.7 | 25.1 | 80.2 | 0.9 | 7.6 |
| 22 | 4.1 | 0.7 | 2.0 | 8.6 | 0.6 | 3.2 |
| 23 | 97.2 | 0.4 | 1.0 | 5.1 | 1.9 | 2.8 |
| 24 | 2.3 | 5.6 | 12.4 | 39.5 | 1.8 | 9.6 |

#K_{D} ratio cyno CD33/human CD33 was calculated based on the K_{D} values measured on CHO cells expressing cynomolgus CD33 and human CD33, respectively. ‡K_{D} ratio hu CD3/hu CD33 was calculated based on the K_{D} values measured on Jurkat cells (hu CD3) and the mean K_{D} of three human CD33⁺ tumor cell lines (HL-60, KG-1, U937).

CD3 binding affinity and crossreactivity were evaluated in titration and flow cytometric experiments on CD3⁺ Jurkat cells (provided by Dr. Moldenhauer, DKFZ Heidelberg; human acute T-cell leukemia) and the cynomolgus CD3⁺ HSC-F cell line (JCRB, cat.: JCRB 1164). CD33 binding and crossreactivity were assessed on the human CD33⁺ tumor cell lines: HL-60 (DSMZ, cat.: ACC 3, human B cell precursor leukemia), U-937 (DSMZ, cat.: ACC5; human histiocytic lymphoma), and KG-1 (DSMZ, cat.: ACC 14; acute myeloid leukemia). The K_{D} ratio of crossreactivity was calculated using the K_{D} values determined on the CHO cell lines expressing either recombinant human or recombinant cynomolgus antigens.

The 2×2 T-cell engagers exhibited a relatively high affinity to human CD33⁺ on most of the tested tumor cell lines below 1 nM. Affinities to human CD3 were determined to be equal or less than 2 nM.

### Example 4

### Cytotoxicity Assay

For the cytoxicity assay target cells cultured under standard conditions were harvested, washed and resuspended in diluent C, provided in the PKH67 Green Fluorescent Cell Linker Mini Kit, to a density of 2×10⁷ cells/mL. The cell suspension was then mixed with an equal volume of a double concentrated PKH67-labeling solution and incubated for 2-5 min at RT. The staining reaction was performed by adding an equal volume of FCS and incubating for 1 min. After washing the labeled target cells with complete RPMI medium, cells were counted and resuspended to a density of 2×10⁵ cells/mL in complete RPMI medium. 2×10⁴ target cells were then seeded together with enriched human T-cells as effector cells at an E:T ratio of 5:1, in the presence of increasing concentrations of the indicated 2×2 T-cell engagers in individual wells of a microtiter plate, in a total volume of 200 μL/well. Spontaneous cell death and killing of targets by T-cells in the absence of antibodies were determined for at least three replicates on each plate. After centrifugation the assay plates were incubated for the indicated periods of time at 37° C. in a humidified atmosphere with 5% CO₂. After incubation, cultures were washed once with FACS buffer and then resuspended in 150 μL FACS buffer supplemented with 2 μg/mL PI. The absolute amount of living target cells was measured by a positive green staining with PKH67 and negative staining for PI using a Beckman-Coulter FC500 MPL flow cytometer (Beckman-Coulter) or a Millipore Guava EasyCyte flow cytometer (Merck Millipore). Based on the measured remaining living target cells, the percentage of specific cell lysis was calculated according to the following formula: [1−(number of living targets₍ₛₐₘₚₗₑ₎/number of living targets₍ₛₚₒₙₜₐₙₑₒᵤₛ₎)]×100%. Sigmoidal dose response curves and EC₅₀ values were calculated by non-linear regression/4-parameter logistic fit using the GraphPad Software. The lysis values obtained for a given antibody concentration were used to calculate sigmoidal dose-response curves by 4 parameter logistic fit analysis using the Prism software.

EC₅₀ values were determined in 20-24 hour assay on CD33⁺ U-937 (DSMZ, cat.: ACC5; human histiocytic lymphoma) target cells with enriched human T-cells as effector cells at a ratio of 5:1. Some 2×2 T-cell engagers were also tested in cytotoxicity assays on CD33⁺ KG-1 (DSMZ, cat.: ACC14; acute myeloid leukemia) and HL-60 target cells. Specifically, HL-60 cells were chosen as a model of an AML with relatively high cell surface expression of CD33 (arbitrary MFI [mean±SEM]: 3,133±215; n=3), and KG-1a was chosen as a model of an AML with very limited CD33 expression (arbitrary MFI: 277±11, n=3). Exemplary cytotoxicity data for selected 2×2 T-cell engagers are summarized in Table 9. Additional cytotoxicity data for HL-60 cell lines is found on Table 8, last column.

TABLE 9
In vitro potency of CD33/CD3 2 × 2 T-cell engagers on
different CD33⁺ tumor cell lines:

| 2 × 2 T- | EC₅₀ [pM (pg/mL)] on human CD33⁺ | | | | | | | |
|---|---|---|---|---|---|---|---|---|
| cell | target cell lines | | | | | | | |
| engager | HL-60 | | U-937 | | KG-1 | | mean | |
| 12 | 1.3 | (137) | 0.8 | (84) | 1.2 | (126) | 1.1 | (116) |
| 14 | 1.6 | (168) | 3.6 | (378) | 2.6 | (273) | 2.6 | (273) |
| 16 | 1.5 | (158) | 1.9 | (200) | 1.8 | (189) | 1.7 | (179) |

EC₅₀ values were determined in FACS-based cytotoxicity assays with primary human T-cells as effector cells at an E:T ratio of 5:1 on the indicated target cell lines incubated for 20-24 hours Each 2×2 T-cell engager was tested on each tumor cell line in at least two independent experiments. Mean values are presented.

### Example 5

### Further Cytotoxicity Screening Experiments in Human CD33+ AML Cell Lines at 48 Hours

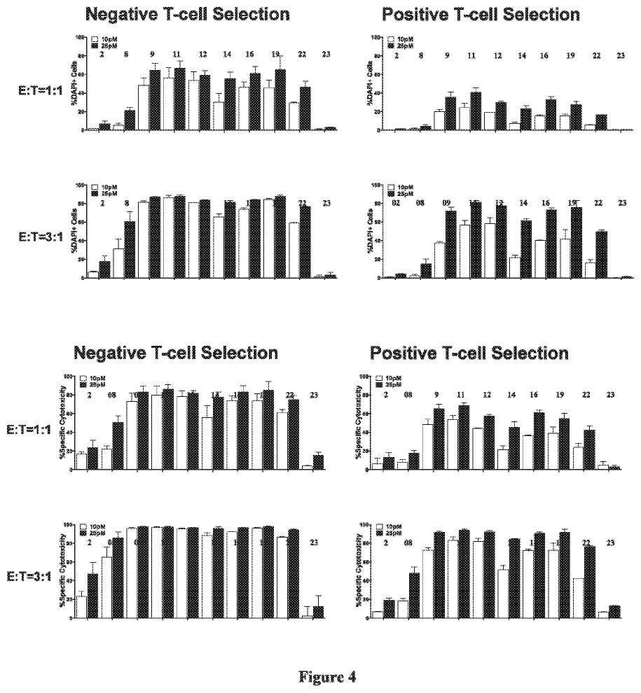

As described above significant cytotoxicity was detected as early as 24 hours, however higher levels of toxicity can be detected at 48 hours. For the subsequent assays a 48-hour time point was chosen. The impact of T-cell selection on 2×2 T-cell engager-induced cytotoxicity was tested. To accomplish this, unstimulated PBMCs from a healthy volunteer donor were obtained, and CD3⁺ cells were isolated both by simple “positive enrichment” via use of CD3 microbeads as well as by more complex “negative selection” via a microbead cocktail of antibodies against CD14, CD15, CD16, CD19, CD34, CD36, CD56, CD123, and CD235a. As depicted in FIG. 4, 2×2 T-cell engager-induced cytotoxicity was greater with negatively selected healthy donor T-cells than positively selected T-cells. However, the relative cytotoxic activities of individual 2-2 T-cell engagers were unaffected by the method of T-cell selection. Therefore the subsequent assays were performed with positively enriched healthy donor T-cells.

Unstimulated mononuclear cells were collected from healthy adult volunteers via leukapheresis by the Fred Hutchinson Cancer Research Center (FHCRC) Hematopoietic Cell Processing Core (Core Center of Excellence) under research protocols approved by the FHCRC Institutional Review Board. T-cells were enriched through magnetic cell sorting either via CD3 Microbeads (“positive enrichment”) or via Pan T-Cell Isolation Kit (“negative selection”; both from Miltenyi Biotec, Auburn, Calif.), and then frozen in aliquots and stored in liquid nitrogen. Thawed cell aliquots were labeled with 3 pM CellVue Burgundy (eBioscience, San Diego, Calif.) according to the manufacturer's instructions. Purified PBMCs were cultured in the presence of various concentrations of 2×2 T-cell engager molecules.

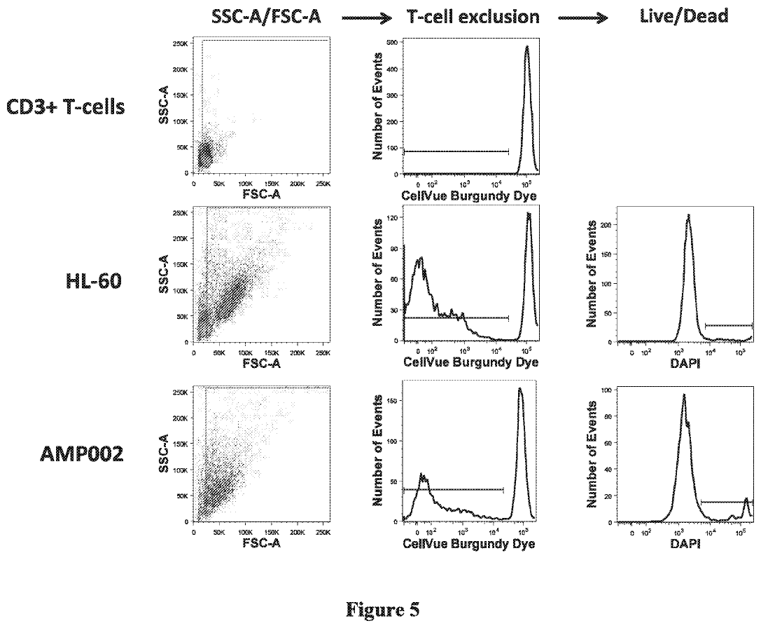

For the quantification of drug-induced cytotoxicity cells were incubated at 37° C. (in 5% CO₂ and air), as in Example 4, at different E:T cell ratios. After 24-72 hours, cell numbers and drug-induced cytotoxicity, using DAPI to detect non-viable cells, were determined using a LSRII cytometer (BD Biosciences) and analyzed with FlowJo. AML cells were identified by forward/side scatter properties and, in experiments where healthy donor T-cells were added, negativity for CellVue Burgundy dye (FIG. 5). Drug-induced specific cytotoxicity is presented as: % cytotoxicity=100×(1−live target cells_{treated}/live target cells_{conrol}). Results from cytotoxicity assays are presented as mean values f standard error of the mean (SEM). Spearman nonparametric correlation was used to compute correlations between continuous sample characteristics. All P-values are two-sided. Statistical analyses were performed using GraphPad Prism software.

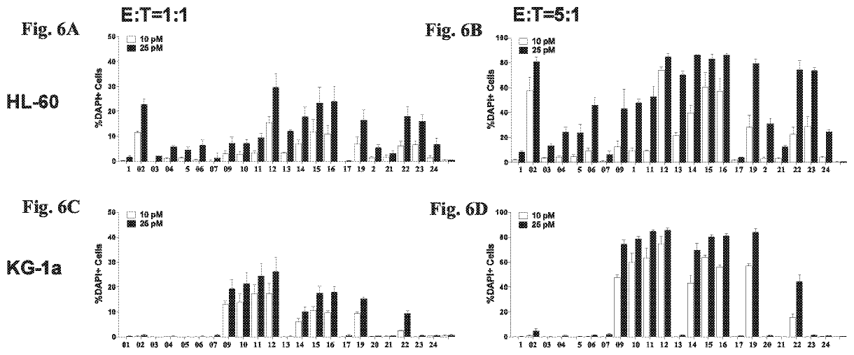

In the absence of healthy donor T-cells, neither of the CD33/CD 2×2 T-cell engagers exerted any noticeable cytotoxic effect on AML cell lines in the absence of T-cells, confirming the absolute requirement for T-cells for their cytotoxic effects (data not shown). In the presence of T-cells, the extent of 2×2 T-cell engager-induced specific cytotoxicity was dependent on the concentration of the 2×2 T-cell engager as well as the E:T cell ratio. Direct head-to-head comparisons between the CD33/CD3-directed 2×2 T-cell engager molecules and one control 2×2 T-cell engager (00) indicated considerable differences in antibody-induced cytotoxicity in both HL-60 cells (FIG. 6A/B and Table 10) and KG-1a cells (FIG. 6C/D and Table 10), with results being highly reproducible in repeat experiments. Overall, the degree of 2×2 T-cell engager-induced cytotoxicity correlated with the binding affinity for CD3 on primary human T-cells (for cytotoxicity in KG-1a cells at 25 pM (approx. 2.5 ng/mL) and E:T=5:1: r=−0.542, p=0.009; for cytotoxicity in HL-60 cells at 25 pM and E:T=5:1: r=−0.391, p=0.07). The 2×2 T-cell engagers 12, 14, 16 were highly cytotoxic for both HL-60 and KG-1a cells.

TABLE 10
CD25 and CD69 induction and cytotoxicity at 48 h of C33/CD3 2 × 2 T-cell
engagers

| | CO3 K_{D} | CD33 K_{D} | CD25 | CD69 | T cell | | |
|---|---|---|---|---|---|---|---|
| 2 × 2 | (nM) | (nM) | Induction | Induction | Proliferation | Cytotoxicity | Cytotoxicity |
| T-cell | Human | HL-60 | EC₅₀ | C₅₀ | in PBMC | HL-60 cells | KG-1a cells |
| engager¹ | T-cells | cells | (pM)² | E(pM)² | EC₅₀ (pM)³ | (% ± SEM)⁴ | (% ± SEM)⁴ |
| 15 | 1.3 | 0.4 | 6 | 7 | 7 | 82.9 ± 3.7 | 80.2 ± 1.9 |
| 12 | 1.5 | 0.3 | 6 | 3 | 2 | 84.7 ± 2.3 | 85.6 ± 1.6 |
| 10 | 1.9 | 0.5 | 10 | 6 | 6 | 48.0 ± 2.4 | 78.6 ± 2.3 |
| 14 | 2.1 | 0.3 | 10 | 7 | 6 | 86.0 ± 0.4 | 69.8 ± 5.7 |
| 21 | 2.1 | 9.7 | ND | 225 | 500 | 12.4 ± 1.0 | 0.0 ± 0.2 |
| 24 | 2.3 | 5.6 | ND | 57 | 264 | 24.5 ± 1.9 | 1.1 ± 0.2 |
| 09 | 2.4 | 0.5 | 11 | 7 | 9 | 43.2 ± 15.8 | 74.6 ± 3.2 |
| 11 | 2.6 | 0.3 | 11 | 5 | 6 | 52.7 ± 8.1 | 84.7 ± 1.4 |
| 17 | 3.3 | 5.0 | 30 | 114 | 30 | 4.2 ± 0.2 | 0.7 ± 0.4 |
| 22 | 4.1 | 0.7 | 10 | 4 | 7 | 74.2 ± 7.4 | 44.4 ± 5.3 |
| 16 | 5.1 | 0.3 | 1 | 2 | 3 | 86.0 ± 1.4 | 81.3 ± 1.5 |
| 19 | 6.3 | 2.8 | 9 | 5 | 6 | 79.4 ± 3.5 | 83.8 ± 2.9 |
| 07 | 49.7 | 13.7 | 134 | 65 | 50 | 6.3 ± 3.3 | 2.1 ± 0.7 |
| 13 | 55.7 | 0.2 | 30 | 22 | 23 | 70.4 ± 2.5 | 1.3 ± 0.4 |
| 05 | 69.5 | 1 | 116 | 74 | 74 | 23.8 ± 6.9 | 0.3 ± 0.3 |
| 02 | 69.8 | 0.2 | 42 | 27 | 4 | 80.9 ± 3.6 | 4.6 ± 2.1 |
| 04 | 79.3 | 0.5 | 94 | 62 | 44 | 24.1 ± 4.0 | 0.7 ± 0.8 |
| 03 | 81.9 | 1.1 | 117 | 87 | 63 | 13.1 ± 3.6 | 0.0 ± 0.5 |
| 06 | 86.3 | 0.4 | 39 | 21 | 48 | 45.7 ± 6.4 | 1.4 ± 0.2 |
| 01 | 94.2 | 0.6 | 92 | 91 | 89 | 8.0 ± 1.6 | 0.4 ± 0.4 |
| 23 | 97.2 | 0.4 | 41 | 17 | 37 | 73.7 ± 2.6 | 1.5 ± 0.3 |
| 20 | 143.8 | 4.1 | 98 | 75 | 38 | 31.2 ± 3.9 | 1.1 ± 0.3 |

¹2 × 2 T-cell engagers are listed in order of increasing CD3 affinity.
²CD25 and CD69 induction was measured after 24 hours in unfractionated PBMC cultures.
³T cell proliferation induced by CD33/CD3 2 × 2 T-cell engager in unfractionated PBMC with CD33+ cells present.
⁴Cytotoxicity (%) after 48 hours of DAPI+ cells at a 2 × 2 T-cell engager concentration of 25 pM in the presence of healthy donor T-cells at an E:T cell ratio of 5:1 from 3 independent experiments performed in duplicate wells.
ND: no CD25 activation detectable

### Example 6

### Further Characterization of 2×2 T-Cell Engagers in Primary Human AML Specimens

For a comprehensive characterization of the cytotoxic properties of these candidates, specimens from AML patients were obtained for the studies from a FHCRC specimen repository.

Frozen aliquots of Ficoll-isolated mononuclear cells from pretreatment (“diagnostic”) peripheral blood or bone marrow specimens from adult patients with AML were obtained from repositories at FHCRC. We used the 2008 WHO criteria to define AML (Vardiman et al.; Blood. 2009; 114(5):937-951) and the refined United Kingdom Medical Research Council (MRC) criteria to assign cytogenetic risk (Grimwalde et al., Blood. 2010; 116(3):354-365). Patients provided written informed consent for the collection and use of their biospecimens for research purposes under protocols approved by the FHCRC Institutional Review Board. Clinical data were de-identified in compliance with Health Insurance Portability and Accountability Act regulations. After thawing, cells were stained with directly labeled antibodies recognizing CD33 (clone P67.6; PE-Cy7-conjugated), CD3 (clone SK7; PerCP-conjugated), CD34 (clone 8G12; APC-conjugated; all from BD Biosciences, San Jose, Calif.), and CD45 (clone HI30; APC-eFluor®780-conjugated; eBioscience). To identify nonviable cells, samples were stained with 4′,6-diamidino-2-phenylindole (DAPI). At least 10,000 events were acquired on a Canto II flow cytometer (BD Biosciences), and DAPI− cells analyzed using FlowJo (Tree Star, Ashland, Oreg.).

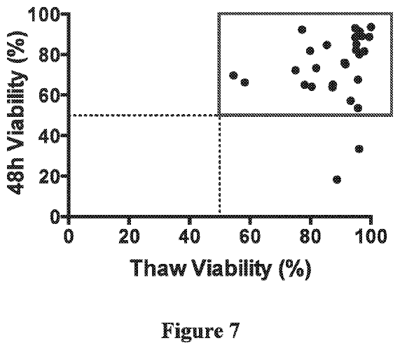

After thawing, specimens had >58% AML blasts, as determined by flow cytometry based on CD45/side-scatter properties. Specimens had >50% viable cells immediately after thawing and >50% viable cells after 48 hours in cytokine-containing liquid culture (FIG. 7). Median age of the patients was 58.1 (range: 23.9-76.2) years; cytogenetic disease risk was favorable in 2, intermediate in 18, and adverse in 7. Information on the mutation status of NPA1, FLT3, and CEBPA was incomplete; however, one sample was known to be CEBPA^{double-mutant}, and another sample was NPMP1^{pos}/FLT3-ITD^{neg}. The median percentage of myeloid blasts and CD3⁺ T-cells in the studied specimens was 86.1% (range: 58.4-97.0%) and 2.0% (range: 0-11.9%), respectively, and the median sample viability after 48 hours in culture was 80.1% (range: 53.6-93.6%). Fifteen of the patients had newly diagnosed AML, whereas 12 either had relapsed (n=7) or refractory (n=5) disease at the time of specimen collection. As summarized in Table 11, basic characteristics of the specimens from patients with newly diagnosed AML were similar to those with relapsed/refractory disease with regard to CD33 expression on myeloid blasts, amount of autologous T-cells, proportion of myeloid blasts, and culture viability.

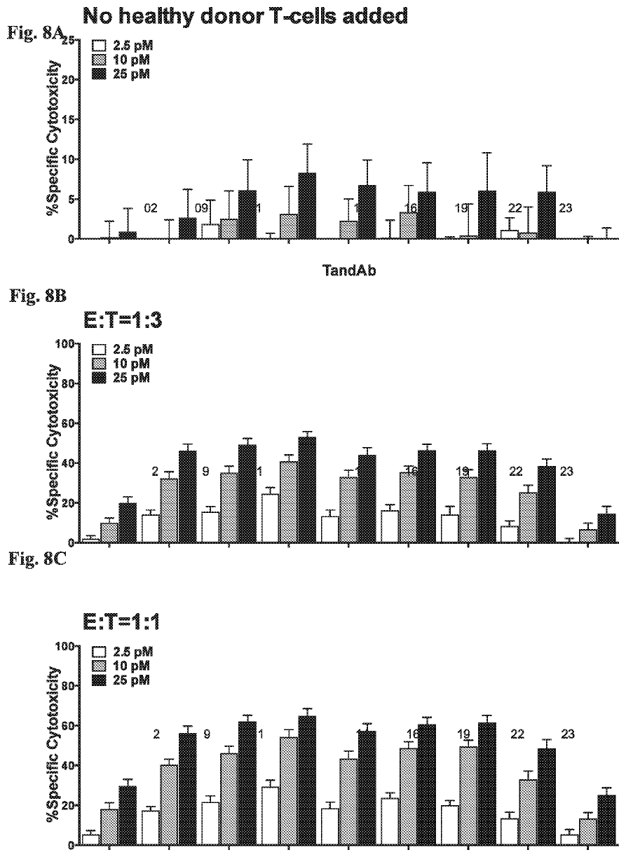

The addition of 2×2 T-cell engager molecules to AML specimen cultures resulted in modest, dose-dependent cytotoxicity (FIG. 8A), demonstrating that autologous T-cells, contained in the specimens from patients with active AML, can be engaged to lyse leukemic cells. In the presence of healthy donor T-cells, the cytotoxic activity of individual 2×2 T-cell engagers was strictly dependent on the drug dose and the E:T cell ratio (FIG. 8B/C). However, high activity of 2×2 T-cell engagers was observed even in some specimens with very low CD33 expression on AML blasts. Among the 2×2 T-cell engager molecules, 12 appeared to be the most active, since it had the highest cytotoxicity at low concentrations (2.5 pM (approx. 250 ng/mL) and, to a less pronounced degree, also 10 pM (approx. 1 ng/mL)) at both E:T=1:3 and E:T=1:1.

The CD33/CD3 2×2 T-cell engagers have been screened in representative AML patient blood samples, which varied in terms of patient sex, age, disease stage (newly diagnosed, relapsed, refractory), degree of CD33 expression and cytogenic risk (Table 11). Remarkably, a number of examined 2×2 T-cell engagers (e.g., 02, 08, 09, 11, 12, 14, 16, 19, 22 and 23) were highly active in nearly all patient samples across the disease spectrum as shown in FIG. 17. Moreover, the extent and scope of activity is similar in all stages of AML, including newly-diagnosed, relapsed and refractory patients.

TABLE 11
Characteristics of primary AML specimens

| | All patients | | Newly diagnosed | | Relapsed/refractory | |
|---|---|---|---|---|---|---|
| | (n = 27) | | AML (n = 15) | | AML (n = 12) | |
| Median age (range), years | 58.1 | (23.9-76.2) | 64.0 | (40.2-76.2) | 44.4 | (23.9-67.4) |
| Cytogenetic/molecular risk | | | | | | |
| Favorable | 2 | | 2 | | — | |
| Intermediate | 18 | | 10 | | 8 | |
| CEBPA^{double-mutant} | 1 | | 1 | | — | |
| NPM1^{pos}/FLT3-ITD^{neg} | 1 | | — | | 1 | |
| NPM1^{pos}/FLT3-ITD^{pos} or | 10 | | 5 | | 5 | |
| NPM1^{neg}/FLT3-ITD^{pos} | | | | | | |
| Adverse | 7 | | 3 | | 4 | |
| Specimen source | | | | | | |
| Bone marrow | 11 | | 4 | | 7 | |
| Peripheral blood | 16 | | 11 | | 5 | |
| Median % blasts (range) | 86.1 | (58.4-97.0) | 86.1 | (66.7-95.5) | 86.7 | (58.4-97.0) |
| Median CD33 expression on blasts (range) | 849 | (5-5,356) | 849 | (5-5,356) | 788 | (7-2,242) |
| Median % T-cells (range) | 2.0 | (0-11.9) | 1.6 | (0-11.9) | 2.1 | (0.7-8.7) |
| Median % viability at 48 hours (range) | 80.1 | (53.6-93.6) | 76.0 | (53.6-93.6) | 83.5 | (63.9-93.1) |

### Example 7

### Potency and Efficacy of CD33/CD3 2×2 T-Cell Engager 12 and 2×2 T-Cell Engager 16 on Different CD33⁺ Cell Lines of Various Origin Expressing Different Levels of CD33

In order to assess whether potency and efficacy of CD33/CD3 2×2 T-cell engagers depend on the CD33 density on the target cells, various human CD33⁺ tumor cell lines and CHO cells expressing recombinant human CD33 were tested for their CD33 expression levels using the QIFIKIT quantification kit and anti-CD33 mAb WM53. The results in Table 12 show that the CD33 densities on the tumor cell lines were in the range between ˜1300 SABC (standardized antibody binding capacity) and ˜46000 SABC. The expression on CHO-CD33 cells was ˜197000 SABC, substantially higher than on the tumor cell lines. All tested CD33⁺ cell lines were used as target cells in at least 3 independent FACS-based cytotoxicity assays with human T-cells as effector cells at an effector-to-target ratio of 5:1 in the presence of serial dilutions of CD33/CD3 2×2 T-cell engager 12 and 2×2 T-cell engager 16. In each assay EC₅₀ and 2×2 T-cell engager-mediated lysis values were calculated by non-linear regression. The results demonstrate that neither the potency (EC₅₀ values) nor the efficacy (% lysis) of 12 and 16 correlates with the CD33 density on the surface of target cells.

Noteworthy, at least 12 and 16 exhibit their cytotoxic activity also against cells like SEM with very low CD33 densities of below 1500 SABC.

TABLE 12
CD33 target cell surface expression and cytotoxic potency of CD33/CD3 2 × 2
T-cell engager 12 and 2 × 2 T-cell engager 16:

| | CD33 density | | 12 | | 16 | |
|---|---|---|---|---|---|---|
| | [SABC] | | EC₅₀ [pM] | | EC₅₀ [pM] | |
| Cell line | mean | SD | mean | SD | mean | SD |
| CHO-CD3:3 | 196990 | 28053 | 11.8 | 11.2 | 24.0 | 19.5 |
| HL-60 | 45948 | 4478 | 1.4 | 0.5 | 1.6 | 0.4 |
| KG-1 | 42828 | 6923 | 1.0 | 0.6 | 1.9 | 2.0 |
| KASUMI-1 | 25922 | 6484 | 1.3 | 0.6 | 2.4 | 1.4 |
| THP-1 | 22065 | 415 | 1.9 | 0.2 | 6.0 | 1.2 |
| RPMI-8226 | 19931 | 2604 | 14.0 | 17.8 | 2.8 | 2.0 |
| U-937 | 17669 | 4593 | 0.9 | 0.1 | 1.3 | 0.6 |
| K562 | 13789 | 2156 | 4.5 | 1.3 | 4.8 | 2.7 |
| BV-173 | 8518 | 1231 | 1.4 | 0.6 | 3.2 | 1.6 |
| SEM | 1306 | 144.2 | 2.2 | 0.5 | 5.1 | 3.0 |

The standardized antibody binding capacity (SABC) on CD33⁺ cell lines was determined using QIFIKIT and the anti-CD33 mAb WM53. EC₅₀ values for 2×2 T-cell engager 12 and 2×2 T-cell engager 16 redirected target cell lysis were determined in FACS-based cytotoxicity assays with human primary T-cells as effector cells at E:T ratios of 5:1 and 20-24 h incubation; assays with CD33-expressing CHO cells were incubated for 40-48 h. Mean and SD of at least 3 independent assays are shown.

### Example 8

### 2×2 T-Cell Engager-Activation of T-Cells and In Vitro Killing of AML Cells

2×2 T-cell engagers were incubated with purified human T cells and a VPD-450-labeled human CD33⁺ leukemia cell line, KG-1, or the CD33⁻ human ALL cell line, G2 (E:T 5:1). Flow cytometry was used to evaluate target cell lysis by 2×2 T-cell engagers (10⁻¹⁵ to 10⁻⁸M; 24 h, 37° C.).

Incubation of 2×2 T-cell engagers 12, 16, and 19 with human T cells efficiently lysed KG-1 cells (IC50 ˜0.01, 0.5, and 5 pM respectively). Up to 40% of T cells were activated (CD25+) rising with cytotoxic activity. A control 2-2 T-cell engager with an irrelevant target, 00 (>10⁻⁷ M), did not result in significant killing of KG-1 in vitro. Separately, 16 induced lysis of KG-1 cells (IC50=5×10⁻¹²M) while 1×10⁻⁸M had no effect on CD33-G2 cells. The results indicate thats T cells become activated and potently lyse tumor cells when targeted to CD33+ leukemic cells (KG-1) and primary CD33+ AML blasts by CD33/CD3 2×2 T-cell engagers.

### Example 9

### Epitope Mapping

2×2 T-cell engagers containing different CD33 binding moieties were subjected to epitope mapping using CLIPS Technology (Pepscan) in order to identify CD33-binding epitopes.

CLIPS Technology facilitates the structuring of peptides into single loops, double-loops, triple loops, sheet-like folds, helix-like folds, and combinations thereof, offering the possibility to map discontinuous epitopes of the target molecule.

An array of more than 7000 independent peptides was synthesized and the binding of each antibody to the peptides was tested in an ELISA.

The 2×2 T-cell engagers 12, 14, 16 and 22 bind to the stretch ₆₂DQEVQEETQ₇₀ (SEQ ID NO:94) in the first Ig like domain of human CD33. The respective amino acid stretches are shown underlined and in bold in FIGS. 10 and 11. It is contemplated that 2×2 T-cell engagers 01, 02, 04, 06, 08, 09, 13 and 23 also bind to this epitope as these 2×2 T-cell engagers share the same CD33 binding domains (SEQ ID NOs:2 and 12, 3 and 13, 5 and 15, 9 and 19) as 2×2 T-cell engagers 12, 14 16 and 12.

### Example 10

### Dose-Response in a Prophylactic In Vivo Tumor Model

2×2 T-cell engagers 12 and 16 are compared at different dose levels in a prophylactic HL-60 tumor xenograft model in NOD/scid mice reconstituted with human T-cells. In order to achieve a dose-response three dose levels at 10, 1 and 0.1 μg (0.5, 0.05, and 0.005 mg/kg) were selected.

Eight experimental groups of immunodeficient NOD/scid mice were xenotransplanted by subcutaneous injection with a suspension of 4×10⁶ HL-60 cells. Prior to injection cells were mixed with 3×10⁶ T-cells isolated from buffy coats (healthy donors) employing negative selection. To account for potential donor variability of the T-cells, each of the experimental groups was subdivided into three cohorts each receiving T-cells of one individual donor only. All animals of the experimental groups transplanted with tumor cells and T-cells received an intravenous bolus on days 0, 1, 2, 3 and 4 (qdxd5) of either vehicle (control) or 16 or 12 at three different dose levels as indicated (0.1 μg, 1 μg, and 10 μg). One group without effector cells and vehicle treatment served as an additional control. Table 13 summarizes group allocation and dosing schedule.

TABLE 13

| Group | treatment | dose | Cell concentration/animal | Cohort | Schedule (iv) | n |
|---|---|---|---|---|---|---|
| 1 | Vehicle | — | 4 × 106 HL-60 | | | 4 |
| 2 | Vehicle | — | 4 × 10⁶ HL-60 + 3 × 10⁶ T-cells | Cohort 1 | Day 0, 1, 2, | 3 |
| | | | 4 × 10⁶ HL-60 + 3 × 10⁶ T-cells | Cohort 2 | 3, 4 | 3 |
| | | | 4 × 10⁶ HL-60 + 3 × 10⁶ T-cells | Cohort 3 | | 3 |
| 3 | 16 | 10 μg | 4 × 10⁶ HL-60 + 3 × 10⁶ T-cells | Cohort 1 | Day 0, 1, 2, | 3 |
| | | | 4 × 10⁶ HL-60 + 3 × 10⁶ T-cells | Cohort 2 | 3, 4 | 3 |
| | | | 4 × 10⁶ HL-60 + 3 × 10⁶ T-cells | Cohort 3 | | 3 |
| 4 | 16 | 1 μg | 4 × 10⁶ HL-60 + 3 × 10⁶ T-cells | Cohort 1 | Day 0, 1, 2, | 3 |
| | | | 4 × 10⁶ HL-60 + 3 × 10⁶ T-cells | Cohort 2 | 3, 4 | 3 |
| | | | 4 × 10⁶ HL-60 + 3 × 10⁶ T-cells | Cohort 3 | | 3 |
| 5 | 16 | 0.1 μg | 4 × 10⁶ HL-60 + 3 × 10⁶ T-cells | Cohort 1 | Day 0, 1, 2, | 3 |
| | | | 4 × 10⁶ HL-60 + 3 × 10⁶ T-cells | Cohort 2 | 3, 4 | 3 |
| | | | 4 × 10⁶ HL-60 + 3 × 10⁶ T-cells | Cohort 3 | | 3 |
| 6 | 12 | 10 μg | 4 × 10⁶ HL-60 + 3 × 10⁶ T-cells | Cohort 1 | Day 0, 1, 2, | 3 |
| | | | 4 × 10⁶ HL-60 + 3 × 10⁶ T-cells | Cohort 2 | 3, 4 | 3 |
| | | | 4 × 10⁶ HL-60 + 3 × 10⁶ T-cells | Cohort 3 | | 3 |
| 7 | 12 | 1 μg | 4 × 10⁶ HL-60 + 3 × 10⁶ T-cells | Cohort 1 | Day 0, 1, 2, | 3 |
| | | | 4 × 10⁶ HL-60 + 3 × 10⁶ T-cells | Cohort 2 | 3, 4 | 3 |
| | | | 4 × 10⁶ HL-60 + 3 × 10⁶ T-cells | Cohort 3 | | 3 |
| 8 | 16 | 0.1 μg | 4 × 10⁶ HL-60 + 3 × 10⁶ T-cells | Cohort 1 | Day 0, 1, 2, | 3 |
| | | | 4 × 10⁶ HL-60 + 3 × 10⁶ T-cells | Cohort 2 | 3, 4 | 3 |
| | | | 4 × 10⁶ HL-60 + 3 × 10⁶ T-cells | Cohort 3 | | 3 |

Treatment groups for the in vivo dose-response study in a HL-60 xenograft model. All animals in the control groups reliably developed a tumor and exhibited homogeneous tumor growth. The presence of T-cells had no influence on tumor development. No difference in HL-60 growth was observed in the presence or absence of T-cells in the vehicle-treated control groups.

Treatment with both test items revealed a clear dose-dependent anti-tumor effect (FIG. 12). No substantial difference was found between the two 2×2 T-cell engagers. Plotting of mean tumor volumes in FIG. 12 was restricted to day 29 when most of the treatment groups were complete. The study was continued until day 45 and animals were observed for tumor-free survival. In the groups treated with 10 or 1 pg of 16, 6 of 9 animals were tumor-free at the end of the observation period and 5 of 9 animals receiving 10 pg of 12 were tumor-free on day 45. One animal remained tumor-free when treated with 1 pg of 12.

All animals in the control groups reliably developed a tumor and exhibited homogeneous tumor growth. Treatment with either of the 2×2 T-cell engagers revealed a dose-dependent anti-tumor effect and no substantial difference was found between the two 2-2 T-cell engagers until day 29.

Detectable differences were observed only after prolonged observation (day 45), at which time the low dose and control groups had already been terminated due to the growth of large tumors. Groups treated with 16 had more tumor-free animals.

### Example 11

### Established Tumor Model

A xenograft model in NOD/scid mice with pre-established HL-60 tumors employing 16 was developed to demonstrate proof of concept.

In brief, female immune-deficient NOD/scid mice were sub-lethally irradiated (2 Gy) and subcutaneously inoculated with 4×10⁶ HL-60 cells. On day 9 the animals received a single bolus injection of anti-asialo GM1 rabbit antibody (Wako, Neuss, Germany) to deplete murine natural killer (NK) cells. On day 10, when the tumor reached a volume between 50-150 mm³ (mean 73±11 mm³) animals were allocated to 3 treatment groups. Groups 2 and 3 (8 animals each) were intraperitoneally injected with 1.5×10⁷ activated human T-cells. Prior to injection T-cells were isolated from buffy coats (healthy donors) employing negative selection. T-cells were expanded and activated with the T-Cell Activation/Expansion Kit according to the manufacturer's specification (Miltenyi Biotech). In order to address potential donor variability Groups 2 and 3 were subdivided into two cohorts each receiving expanded and activated T-cells from an individual donor. Each cohort received T-cells from one individual T-cell donor only.

TABLE 14
Treatment groups for the established HL-60 xenograft model.

| | Animals | Inoculated cells | | | Treatment |
|---|---|---|---|---|---|
| Group | (n) | Day 0, sc. | Day 10, ip. | Cohort | Day 13 to 21, once daily |
| 1 | 5 | 4 × 10⁶ HL-60 | | | Vehicle (iv) |
| 2 | 4 | 4 × 10⁶ HL-60 | 1.5 × 10⁷ T-cells (Donor 1) | 1 | Vehicle (iv) |
| | 4 | 4 × 10⁶ HL-60 | 1.5 × 10⁷ T-cells (Donor 2) | 2 | |
| 3 | 4 | 4 × 10⁶ HL-60 | 1.5 × 10⁷ T-cells (Donor 1) | 1 | 2 × 2 T-cell engager 16 |
| | 4 | 4 × 10⁶ HL-60 | 1.5 × 10⁷ T-cells (Donor 2) | 2 | (iv) 50 μg |

Starting on day 13 animals in Group 3 displayed a mean tumor volume of 105 mm³ and were treated with a total of 9 intravenous doses of 50 pg 2×2 T-cell engager 16 (qdx9d). Table 14 illustrates group allocation and dosing schedule. Groups 1 and 2 were only treated with the vehicle. Body weight and tumor volume were determined until day 27.

All animals reliably developed a tumor, which was palpable on day 6. The mean tumor volume of vehicle-treated Group 1 and 2 (HL-60) animals continually increased until study termination on day 27 (FIG. 13). In Group 2 animals that received primary activated human T-cells in addition to HL-60 tumor cells, the mean tumor volume increased faster compared to Group 1 (HL-60 only).

Repeated intravenous treatment from days 13 to 21 (qdxd9) with 2×2 T-cell engager 16 (50 μg/animal; 2.5 mg/kg) in the presence of human T-cells (Group 3) rapidly delayed tumor growth relative to Group 1 and Group 2. 2×2 T-cell engager 16 delayed tumor growth in Group 3 by approximately 4-5 days compared to vehicle-treated control group (Group 2). Statistically significant differences in the time period from day 6 to day 27 were identified between Group 2 (HL-60, T-cells, vehicle) and Group 3 (HL-60, T-cells, 16) on day 22 (p<0.05), day 23 (p<0.01) and day 27 (p<0.01) (Two-way Repeated Measures ANOVA with Bonferroni post-tests). No statistically significant differences were present between Group 1 and Group 3 due to unusual slow growth of the tumor in Group 1.

No donor variability with regard to T-cell activity was observed, when comparing tumor development in Cohort 1 and Cohort 2 within a group, which received T-cells from different donors (see Table 14).

Example 10 shows that a xenograft model in NOD/scid mice with a pre-established HL-60 tumor (AML) and intraperitoneally-engrafted human T-cells was successfully developed. Repeated dosing with 2×2 T-cell engager 16 at a single dose level lead to a statistically significant delay in tumor growth in comparison to the respective vehicle-treated control group. The data generated are comparable to results published for a similar study with a CD33/CD3 BiTE™ (Aigner el al., 2012; Leukemia, 2013, April; 27(5):1107-15).

### Example 12

### Efficacy of CD33/CD3 2.2 T-Cell Engagers in an AML PDX Model in NSG Mice

Cryopreserved cells from an AML patient whose CD33˜ leukemia contained 2-4% CD3⁺ T-cells were used to establish an AML PDX model in NSG mice. One hour post-injection of tumor cells into irradiated (250 cGy) NSG mice, CD33/CD3 2×2 T-cell engagers, 16 or 12, at either of two i.v. doses (50 μg or 5 μg; n=8 mice/group) were injected in a 200 μL bolus. Additional injections of 2×2 T-cell engagers were performed on each of the following 4 days. Mice were weighed once weekly, and subsequently were sacrificed on day 38 to permit collection of peripheral blood, bone marrow, and spleen for analysis by flow cytometry (huCD33, huCD34, huCD45, muCD45, huCD14, huCD3, huCD4, huCD8, and 7AAD). The results are shown in FIG. 14.

FIG. 14 shows that untreated mice had substantial amounts of human blasts in the bone marrow and spleen after 38 days. In contrast, mice treated with daily i.v. injections of 2×2 T-cell engagers 12 or 16 exhibited substantially lower numbers of human AML blasts in the bone marrow and in the spleen. The strong anti-AML effect of the CD33/CD3 2×2 T-cell engager was observed at both dose levels (5 and 50 pg/injection).

The observed anti-AML effect for both CD33/CD3 2×2 T-cell engagers, 12 and 16, was much stronger than the effect of a CD123/CD3 DART® antibody targeting AML in an identical mouse model (Hussaini et al.: “Targeting CD123 In Leukemic Stem Cells Using Dual Affinity Re-Targeting Molecules (DARTs®) Nov. 15, 2013; Blood: 122 (21)). In contrast to the CD33/CD3 2×2 T-cell engagers which eliminated nearly all AML blasts in bone marrow and spleen, Hussaini et al. reported that the CD123/CD3 DART® reduced the number of AML blasts in the bone marrow and spleen in the PDX model only by factor 50-1000 at 2.5 and 0.25 mg/kg, the authors further reported that the CD123/CD3 DART™ reduced the number of AML blasts in bone marrow and spleen in the PDX model only by 40-78% at 0.5 mg/kg.

### Example 13

### Fast Onset of CD33/CD3 2×2 T-Cell Engager 16-Mediated Target Cell Lysis

In order to assess the kinetics of CD33/CD3 2×2 T-cell engager-mediated target cell lysis, calcein-release cytotoxicity assays with different incubation times were performed. Calcein-labeled CD33⁺ HL-60 target cells were incubated with serial dilutions of 2×2 T-cell engager 16 in the presence of primary human T cells as effector cells at an E:T ratio of 25:1 for 30 min, 1 h, 2 h, 3 h, 4 h, or 5 h. At each time point the calcein that was released from lysed target cells was used to calculate the EC₅₀ value and 2×2 T-cell engager 16-mediated target cell lysis using non-linear regression/sigmoidal dose-response. FIG. 15 shows an unexpected fast onset of 2×2 T-cell engager-mediated target cells lysis with more than 40% lysis after 30 min incubation at saturating 2×2 T-cell engager concentrations. After 4 hours incubation more than 90% target cell lysis was reached. Table 15 and FIG. 16 summarize the EC₅₀ and specific lysis values determined for 2×2 T-cell engager 16 at incubation times between 30 min and 5 hours. The results further demonstrate that under the used assay conditions maximal potency (lowest EC₅₀ value) was reached after 2 hours incubation and that after 5 hours incubation almost all target cells were lysed. Altogether these results demonstrate a very fast, potent and efficacious target cell lysis mediated by CD33/CD3 2×2 T-cell engagers.

TABLE 15
Kinetics of EC₅₀ and lysis values determined for 2 × 2 T-cell engager 16

| incubation | | 2 × 2 T-cell engager- |
|---|---|---|
| time [min] | EC₅₀ [pM] | mediated lysis [%] |
| 30 | 4.8 | 44.1 |
| 60 | 2.5 | 59.8 |
| 120 | 1.6 | 75.1 |
| 180 | 1.6 | 88.8 |
| 240 | 1.5 | 93.7 |
| 300 | 1.6 | 97.4 |

### Example 14

### Clinical Trial Protocol for Dose Escalation Study of a CD33/CD3 2×2 T-Cell Engager to AML Patients

This is a Phase I clinical trial to characterize the safety and tolerability of CD33/CD3 2×2 T-cell engager 16 (AMV 564).

Study Outcomes:

Primary: Dose Escalation Stage: 1) To characterize the safety and tolerability, including dose-limiting toxicity (DLT), of CD33/CD3 2×2 T-cell engager 16 when administered via continuous intravenous infusion; 2) To identify the maximum tolerated dose (MTD) and recommended Phase 2 dose (RP2D)

Secondary: To characterize the pharmacokinetics (PK), pharmacodynamics (PD), and immunogenicity of CD33/CD3 2×2 T-cell engager 16 when administered continuous intravenous infusion;

Study Design: This study is a first in human, Phase 1, open label, multicenter, dose escalation study with expansion at the RP2D, to evaluate the safety, tolerability and preliminary antileukemic activity of CD33/CD3 2×2 T-cell engager 16 in patients with relapsed or refractory acute myeloid leukemia (AML). Approximately 50 patients will be enrolled at approximately 6 centers in the US or EU; the total number of patients will depend on the dose level at which the RP2D is defined.

CD33/CD3 2×2 T-cell engager 16 will be given via CIV administration for a total of 14 days per cycle, for 1 or 2 induction cycles. Patients will undergo bone marrow assessments at Screening, on Day 15 (within 24 hours of end of infusion), on Day 29 (+5 days), and at time of hematologic recovery during each CD33/CD3 2×2 T-cell engager 16 induction cycle, and at other times if clinically indicated.

Dose Escalation Stage

A standard 3+3 dose escalation scheme will be employed to determine the MTD and will follow the scheme outlined in the below table:

| Dose | | | Treatment |
|---|---|---|---|
| Level | Dose | | Regimen* |
| 1 | 0.5 | μg/day | 0.5 μg/d × 14 d |
| 2 | 1.5 | μg/day | 1.5 μg/d × 14 d |
| 3 | 5 | μg/day | 5 μg/d × 14 d |
| 4 | 15 | μg/day | 15 μg/d × 14 d |
| 5 | 50 | μg/day | 50 μg/d × 14 d |
| 6 | 100 | μg/day | 100 μg/d × 14 d |
| 7 | 150 | μg/day | 150 μg/d × 14 d |
| 8 | 200 | μg/day | 200 μg/d × 14 d |
| 9 | 250 | μg/day | 250 μg/d × 14 d |
| 10 | 300 | μg/day | 300 μg/d × 14 d |

Abbreviations:
d = day
If cytokine release syndrome (CRS) is observed in ≥2 patients in the study (at any dose), the Study Evaluation Team (SET) will designate a lead-in dose (lower than that at which CRS was observed) for the first 3 days, followed by 11 days at the assigned dose level

### Eligibility:

a. ≥18 years of age at the time of signing informed consent
b. Diagnosis of AML according to the World Health Organization (WHO) 2008 criteria
c. Relapsed or refractory disease meeting the following criteria: (a) Primary refractory, ie, refractory to induction with a standard anthracycline-based regimen or a hypomethylating agent (e.g. decitabine or azacitidine) for patients ineligible for anthracycline-based therapy; (b) First relapse after a first complete remission (CR) lasting less than 12 months; or (c) Second or later relapse. Relapse is defined as the reappearance of leukemic blasts in the peripheral blood or ≥5% leukemic blasts in the bone marrow after prior achievement of a CR or CRi.
d. No more than 2 prior induction regimens for newly diagnosed AML, no more than 1 prior stem cell transplant, and no more than 2 prior salvage regimens for relapsed or refractory AML. Any number of continuous cycles of therapy with an individual hypomethylating agent count as one induction or salvage regimen.
e. Blasts at least 5% in bone marrow
f. Peripheral white blood cell (WBC) count: no upper limit at Screening, but must be <10×10⁹/L on Day 1 prior to treatment; patients with excessive blasts may be treated with hydroxyurea to bring counts down.
g. Chemistry laboratory parameters within the following range: Aspartate aminotransferase (AST) and alanine aminotransferase (ALT) ≤2× the upper limit of normal (ULN); Total bilirubin ≤1.5× the ULN; patients with Gilbert's syndrome can enroll if conjugated bilirubin is within normal limits, Creatinine clearance >50 mL/min (measured or calculated by Cockcroft-Gault method)
h. Eastern Cooperative Oncology Group (ECOG) performance status of 0 or 1. Patients with ECOG score of 2 may be included, after discussion with the Sponsor Medical Monitor, if score is influenced by symptoms attributable to underlying AML disease.

Preliminary Clinical Pharmacokinetics:

TABLE 16
Pharmacokinetic Parameters of
Three Subjects dosed with AMV 564

| | | Dose | Cmax | Tmax | AUC(0-t) |
|---|---|---|---|---|---|
| SUBJID | Cycle | (mcg/day) | (pg/mL) | (day) | (day*pg/mL) |
| 02-001 | 1 | 0.5 × 14 days | 88.7 | 15.99 | 705 |
| 02-002 | 1 | 0.5 × 14 days | 32.9 | 7.00 | 362 |
| 02-002 | 2 | 0.5 × 14 days | 31.7 | 14.00 | 377 |
| 02-003 | 1 | 0.5 × 14 days | 40.1 | 13.93 | 365 |

FIG. 18 depicts the serum concentration of CD33/CD3 2×2 T-cell engager 16 (AMV 564) in subjects 02-001, -002, and -003 at a dose of 0.5 pg/day in a first 14 day cycle (left panel). Subject 02-002 received a second cycle at the same concentration.

FIG. 19 shows serum concentrations following continuous intravenous administration of AMV564 for 14 days at the 0.5, 1.5, 5, 15, or 50 mcg dose levels to patients with relapsed/refractory acute myeloid leukemia. It was observed that concentrations gradually increase and reach steady-state in 3-7 days. The terminal half-life is approximately 2 days. N=3 per dose level except 50 mcg where N=1. Concentrations were measured from serum collected at specified timepoints using a validated method.

Pharmacodynamics:

In a subject dosed at 0.5 pg/day with AMV 564 for 14 days, it was observed that myeloblast counts were controlled, CRP levels were reduced and increased hematopoiesis was observed as evidenced by sustained and increased hemoglobin and neutrophils (FIG. 20). The subject received a transfusion on day 3 of the treatment.

A similar response was observed in a second subject dosed at 1.5 pg/day with AMV 564 for 14 days which shows dramatic improvement in blood counts (FIG. 21). Neutrophils and erythrocyte counts improved and recovered (upper panels) following administration of AMV564. Blood samples were collected at specified timepoints and processed to determine these blood parameters. Interleukin-6 and C-reactive protein both decreased following administration of AMV564 at a 1.5 mcg dose level for 14 days (lower panels). Samples were collected at specified timepoints. Interleukin-6 was measured in a validated multiplex immunoassay. C-reactive protein was measured using a qualified instrument. It is contemplated that recovery is driven by elimination of MDSCs in the subject.

Additional subjects dosed at 1.5 pg/day with AMV 564 for 14 days attained similar results in blood counts. FIG. 22 (upper panels) shows one subject that had improved hemoglobin, neutrophils, platelet, and monocyte counts following administration and another subject (lower panels) having improved hemoglobin, neutrophils, platelet, and monocyte counts as well as decreased C-reactive protein. Blood samples were collected at specified timepoints and processed to determine these blood parameters. C-reactive protein was measured using a qualified instrument.

### Additional Observations

Antileukemic activity was additionally observed in initial subjects. FIG. 23 depicts best relative change in percent bone marrow leukemic blasts from baseline following administration of AMV564 for 14 days to patients with relapsed/refractory acute myeloid leukemia. Each bar represents an individual patient response. Bone marrow samples were taken periodically during the clinical study period and the percent bone marrow blasts determined by cellular morphology. The x-axis denotes the dose administered in the units of mcg.

In one of the subjects, spleen size was reduced from 18 cm to 11 cm.

### Example 15

Clinical Trial Assessing the Safety and Efficacy of a CD33/CD3 2×2 T-Cell Engager in Adult Patients with Established Rheumatoid Arthritis

This study will assess the safety, efficacy, and response to treatment using the American College of Rheumatology criteria of 20% improvement in symptoms (ACR20) in adult patients with established rheumatoid arthritis using CD33/CD3 2×2 T-cell engager 16 (AMV 564).

Study Type: Interventional

Study Design:

Allocation: Randomized

Control: Placebo Control

Endpoint Classification: Safety/Efficacy Study

Intervention Model: Parallel Assignment

Masking: Double Blind (Subject, Caregiver, Investigator, Outcomes Assessor)

Primary Purpose: Treatment

Primary Outcome Measures: Response to treatment (ACR20) in adult patients with established rheumatoid arthritis (RA) [Time Frame: at 6 weeks]

Eligibility: Ages eligible for study: 18 years to 75 years; Genders eligible for study: Both

Inclusion Criteria:

### Ra Patients:

Male and female patients aged 18-75 years (inclusive); Body weight between 50 and 100 kg (inclusive);
Post menopausal or surgically sterile female patients are allowed.
Female patients of child-bearing potential may participate if they are already on a stable dose of methotrexate. Additional birth control details to be provided at screening.
Male patients must use an effective contraception method during the study and at least for 2 months following the completion/discontinuation of the study;
Diagnosis of RA, classified by American Rheumatism Association 1987 revised criteria. Disease duration of at least 6 months is essential;
Functional status class I, II or III classified according to the American College of Rheumatology 1991 revised criteria;
Active disease evaluation ≥6 tender and ≥6 swollen joints);
Prior treatment with 1-3 disease-modifying anti-rheumatic drugs (DMARDs)—Patients should have failed at least 1 DMARD but should not be deemed “refractory to all therapies”. It is expected that patients are on a current treatment with methotrexate ≤25 mg/week and with the current dose stable for at least 3 months, however patients who did not tolerate MTX may also be considered. All patients will take folic acid 1 mg daily, or 5 mg weekly post MTX dose, to minimize toxicity, according to local guidelines. In addition to methotrexate, patients may be on either a stable dose of non-steroidal anti-inflammatory drugs (NSAIDs) and/or a stable dose of oral corticosteroids (prednisone or equivalent ≤10 mg daily) for at least 4 weeks prior to randomization. Patients who failed any DMARDs will be allowed;

### Example 16

Clinical Trial Assessing the Safety and Efficacy of a CD33/CD3 2×2 T-Cell Engager in Subjects with Moderately to Severely Active Systemic Lupus Erythematosus (SLE)

The purpose of this study is to evaluate the safety and tolerability of CD33/CD3 2×2 T-cell engager 16 (AMV 564) in adult subjects with moderately to severely active systemic lupus erythematosus (SLE)

Study Type: Interventional

Study Design:

Allocation: Randomized

Control: Active Control

Endpoint Classification: Safety Study

Intervention Model: Parallel Assignment

Masking: Double Blind (Subject, Investigator)

Primary Purpose: Treatment

Primary Outcome Measures: The safety and tolerability of a pharmaceutical dosage form of CD33/CD3 2×2 T-cell engager 16 is assessed primarily by summarizing treatment-emergent adverse events (AEs) and serious adverse events (SAEs) [Time Frame: Study Day 169]

Secondary Outcome Measures:] The secondary endpoints of the study are to assess the PK and immune response of doses of a pharmaceutical dosage form of CD33/CD3 2×2 T-cell engager 16 in adult subjects with moderately to severely active SLE. [Time Frame: Study Day 169]

PK: Individual and mean serum concentration-time profiles of a pharmaceutical dosage form of CD33/CD3 2×2 T-cell engager 16 by treatment group generated. Immune response: The presence of anti-drug antibodies against a pharmaceutical dosage form of CD33/CD3 2-2 T-cell engager 16 in serum is assessed and reported by number of subjects with detectable anti-drug antibodies and the percentage of positive subjects by treatment group. The titers of anti-drug antibodies in positive subjects will be reported.

Eligibility: Ages eligible for study: 18 years and older; Genders eligible for study: Both

Inclusion Criteria:
Male or female subjects;
Age≥18 years at the time of screening;
Written informed consent and HIPAA authorization obtained from the subject/legal representative prior to performing any protocol-related procedures, including screening evaluations;
Meet or have met at least 4 of the 11 revised American College of Rheumatology (ACR) classification criteria for SLE (Appendix 2);
Score≥6 points on the Systemic Lupus Erythematosus Disease Activity Index 2000 (SLEDAI-2K) at screening and baseline;
Have positive antinuclear antibody (ANA) test at 21:80 serum dilution at screening;
Have active skin lesions from SLE in at least one area suitable for repeat skin biopsy, such as on the arms, legs, or trunk;
Females of childbearing potential, unless surgically sterile (ie, bilateral tubal ligation, bilateral oophorectomy, or complete hysterectomy), has sterile male partner, or post menopausal (defined as at least 2 years since last regular menses and follicle stimulating hormone (FSH) >23 IU/L according to central lab), or practices abstinence, must use 2 effective methods of avoiding pregnancy (including oral, transdermal, or implanted contraceptives, intrauterine device, female condom with spermicide, diaphragm with spermicide, cervical cap, or use of a condom with spermicide by the sexual partner) from screening, and must agree to continue using such precautions through the Early Discontinuation/End of Study (Day 169) visit; cessation of birth control after this point should be discussed with a responsible physician;
Males, unless surgically sterile, must use 2 effective methods of birth control with a female partner and must agree to continue using such contraceptive precautions from Day 1 through the Early Discontinuation/End of Study (Day 169) visit;
Ability to complete the study period, including follow-up period through Day 169;
Willingness to forego other forms of experimental treatment during the study.

1. A method for the treatment of an inflammatory disease or condition in a subject comprising administering to a subject in need thereof a bivalent, bispecific T-cell engager comprising a polypeptide comprising an amino acid sequence selected from the group consisting of SEQ ID NOs:98-121 and 123-146.

2. The method according to claim 1, wherein the inflammatory disease or condition is an autoimmune disease.

3.-8. (canceled)

9. The method according to claim 1, wherein the inflammatory disease or condition is caused by a pathogenic infection.

10.-46. (canceled)
